(19) United States
(12) Patent Application Publication
(10) Pub. No.: US 2023/0116082 A1
(43) Pub. Date: Apr. 13, 2023

(54) METHODS AND COMPOSITIONS FOR TREATING TISSUE DAMAGE RESULTING FROM VIRAL INFECTIONS

(71) Applicant: NELLONE THERAPEUTICS, INC., OAK RIDGE, TN (US)

(72) Inventors: CYMBELINE T. CULIAT, OAK RIDGE, TN (US); SHANNON STEWART EAKER, KNOXVILLE, TN (US)

(73) Assignee: NELLONE THERAPEUTICS, INC., OAK RIDGE, TN (US)

(21) Appl. No.: 17/908,080

(22) PCT Filed: Mar. 5, 2021
(86) PCT No.: PCT/US2021/021193

§ 371 Date: Aug. 30, 2022

Related U.S. Application Data
(60) Provisional application No. 63/087,279, filed on Oct. 4, 2020.
(60) Provisional application No. 63/008,271, filed on Apr. 10, 2020.

(51) Int. Cl. A61K 38/18; A61P 31/14

(52) U.S. Cl. CPC A61K 38/1808; A61P 31/14

(57) ABSTRACT

Provided are methods of treating virally-induced tissue damage (e.g., lung tissue damage, heart tissue and/or vasculature damage, skeletal muscle damage) and/or inflammation by administering a NELL1 polypeptide, or a nucleic acid molecule encoding a NELL1 polypeptide, to a subject in need thereof. Methods of regenerating lung tissue in a subject are also provided wherein a NELL1 polypeptide or a nucleic acid molecule encoding the same is administered to a subject with damaged lung tissue

### CROSS REFERENCED APPLICATIONS

This application claims the benefit of U.S. Provisional Application Nos. 63/008,271, filed Apr. 10, 2020, and 63/087,279, filed Oct. 4, 2020, each of which is incorporated herein by reference in its entirety.

### REFERENCE TO SEQUENCE LISTING SUBMITTED ELECTRONICALLY

The official copy of the sequence listing is submitted electronically via EFS-Web as an ASCII formatted sequence listing with a file named N88509_1080WO_SL_ST25.txt, created on Mar. 5, 2021, and having a size of 159,521 bytes. The sequence listing contained in this ASCII formatted document is part of the specification and is herein incorporated by reference in its entirety.

### FIELD OF THE INVENTION

This application generally relates to the healing of tissue damage resulting from viral infections with a NELL1 protein or a nucleic acid encoding the same.

### BACKGROUND OF THE INVENTION

Viruses attack and infect many tissues in the human body, eliciting an overreaction of the immune system and direct damage by causing cell death of the tissues. This damage can be so severe that it leads to organ failure and fatalities. In survivors of the infection, disabilities can result. Coronaviruses, in particular, attack the lung and heart tissues of vulnerable patients and collapse the respiratory system via both direct routes by entry and takeover of cell machinery to replicate viral particles or indirect routes by triggering an over-reaction of the immune system generating a cytokine storm that severely inflames and impairs soft tissues (Cascella M et al. 2020 Features, Evaluation and Treatment Coronavirus (COVID-19) on the world wide web at ncbi.nlm.nih.gov/books/NBK554776/; Shereen M A et al. 2020 J of Advanced Research 24:91-98; Singhal T 2020 The Indian Journal of Pediatrics 87(4):281-286; Sarzi-Puttini P et al. 2020 Clin Exp Rheumatol 38:337-342; Tisoncik J R et al. 2012 Microbiology and Molecular Biology Reviews 76(1):16-32).

### SUMMARY OF THE INVENTION

Methods for treating tissue damage and/or inflammation resulting from a viral infection are provided. The methods comprise administering to a subject in need thereof an effective amount of a NELL1 polypeptide or a nucleic acid encoding the same. The infection can be by a respiratory virus, thus affecting cells of the upper and/or lower respiratory system. In some of these embodiments, the tissue damage is damage to a lung tissue (e.g., lung epithelium), such as the alveolar type II cells. In those embodiments wherein the tissue damage is damage to a lung tissue, a NELL1 polypeptide or nucleic acid molecule comprising the same can be administered systemically or via inhalation.

In certain embodiments, the tissue damage is caused by the infection of an enveloped virus, including an enveloped virus that is released by its host cells via exocytosis and generates multinucleated cells to mediate cell-to-cell infection. In some of these embodiments, the enveloped virus is a coronavirus. In certain embodiments, the coronavirus attaches and gains entry into host cells via binding to angiotensin-converting enzyme 2 (ACE2). In some of these embodiments, the coronavirus is severe acute respiratory syndrome coronavirus 2 (SARS-CoV-2).

In certain embodiments, the subject is exhibiting a cytokine storm. In some of these embodiments, the subject has elevated levels of any one of interleukin-6 (IL-6), interferon gamma induced protein 10 (IP-10), monocyte chemotactic protein-3 (MCP-3), interleukin-1ra (IL-1ra), interferon-gamma (IFN-γ), interleukin-2ra (IL-2ra), interleukin-10 (IL-10), interleukin-18 (IL-18), hepatocyte growth factor (HGF), macrophage inflammatory protein 1 alpha (MIG-1a), macrophage colony stimulating factor (M-CSF), granulocyte colony-stimulating factor (G-CSF), and cutaneous T-cell-attracting chemokine (CTACK), when compared to a healthy control subject. In some of these embodiments, the subject has blood levels of interleukin-6 (IL-6) of at least about 80 pg/ml.

In particular embodiments, the subject is administered a NELL1 polypeptide or nucleic acid molecule encoding the same after the subject tests positive for coronavirus disease 2019 (COVID-19) (i.e., infection by SARS-CoV-2) or the subject exhibits symptoms of COVID-19.

In some embodiments, the subject that is administered a NELL1 polypeptide or nucleic acid molecule encoding the same has pneumonia. In particular embodiments, the subject has acute lung injury (ALI) or acute respiratory distress syndrome (ARDS). In certain embodiments, the subject is on supplementary oxygen or artificial ventilation.

In other embodiments, the tissue damage that is treated with a NELL1 polypeptide or nucleic acid molecule is damage to a heart tissue (e.g., cardiomyocytes) or vasculature. In some of these embodiments, a NELL1 polypeptide or nucleic acid molecule is administered systemically or via intraarterial injection. In certain embodiments, the subject has elevated cardiac troponin 1 or troponin T levels when compared to a healthy control subject.

In certain embodiments, the tissue damage that is treated with a NELL1 polypeptide or nucleic acid molecule is damage to skeletal muscle tissue. In some of these embodiments, a NELL1 polypeptide or nucleic acid molecule is administered systemically.

In certain embodiments, the NELL 1 polypeptide has an amino acid sequence having at least 80% sequence identity to the amino acid sequence set forth as SEQ ID NO: 2, 4, 6, 10, or 12. In some of these embodiments, the NELL1 polypeptide is the polypeptide of SEQ ID NO: 2, 4, 6, 10, 12, 17, or 18. In some of those embodiments wherein the method comprises administering a nucleic acid molecule encoding a NELL1 polypeptide, the nucleic acid molecule is comprised within an expression vector and operably linked to a promoter. The subject can be a mammal, such as a human.

Also provided are methods for regenerating lung tissue in a subject comprising administering to a subject with damaged lung tissue an effective amount of a NELL1 polypeptide or a nucleic acid molecule encoding the same. In some of these embodiments, the damaged lung tissue is a result of an infection by a virus. In some of these embodiments, the virus is a respiratory virus. In some embodiments, the virus is an enveloped virus, including an enveloped virus that is released by its host cells via exocytosis and generates multinucleated cells to mediate cell-to-cell infection. In some of these embodiments, the enveloped virus is a coronavirus. In certain embodiments, the coronavirus attaches and gains entry into host cells via binding to ACE2. In some of these embodiments, the coronavirus is SARS-CoV-2.

In some embodiments, the damaged lung tissue is from viral pneumonia. In particular embodiments, the damaged lung tissue is from ALI or ARDS. In certain embodiments, a NELL1 polypeptide or nucleic acid molecule encoding the same is administered via inhalation or systemically.

In certain embodiments, the NELL 1 polypeptide has an amino acid sequence having at least 80% sequence identity to the amino acid sequence set forth as SEQ ID NO: 2, 4, 6, 10, or 12. In some of these embodiments, the NELL1 polypeptide is the polypeptide of SEQ ID NO: 2, 4, 6, 10, 12, 17, or 18. In some of those embodiments wherein the method comprises administering a nucleic acid molecule encoding a NELL1 polypeptide, the nucleic acid molecule is comprised within an expression vector and operably linked to a promoter. The subject can be a mammal, such as a human.

In another aspect, provided herein are methods for treating lung inflammation in a subject comprising administering to a subject in need thereof an effective amount of a NELL1 polypeptide or a nucleic acid molecule encoding the same. In certain embodiments, the lung inflammation is due to an infection by a virus. In some of these embodiments, the virus is a respiratory virus. In some embodiments, the virus is an enveloped virus, including an enveloped virus that is released by its host cells via exocytosis and generates multinucleated cells to mediate cell-to-cell infection. In some of these embodiments, the enveloped virus is a coronavirus. In certain embodiments, the coronavirus attaches and gains entry into host cells via binding to ACE2. In some of these embodiments, the coronavirus is SARS-CoV-2.

In another aspect, provided herein are methods for treating weight loss or muscle atrophy due to a viral infection in a subject in need thereof. The method comprises administering to the subject an effective amount of a NELL1 polypeptide, or a nucleic acid molecule encoding the same. In some embodiments, the viral infection is an infection of a respiratory virus. In some embodiments, the viral infection is an infection of a coronavirus. In certain embodiments, the coronavirus is SARS-CoV-2.

In certain embodiments, the subject is exhibiting a cytokine storm. In some of these embodiments, the subject has elevated levels of any one of interleukin-6 (IL-6), interferon gamma induced protein 10 (IP-10), monocyte chemotactic protein-3 (MCP-3), interleukin-1ra (IL-1ra), interferon-gamma (IFN-γ), interleukin-2ra (IL-2ra), interleukin-10 (IL-10), interleukin-18 (IL-18), hepatocyte growth factor (HGF), macrophage inflammatory protein 1 alpha (MIG-1a), macrophage colony stimulating factor (M-CSF), granulocyte colony-stimulating factor (G-CSF), and cutaneous T-cell-attracting chemokine (CTACK), when compared to a healthy control subject. In some of these embodiments, the subject has blood levels of interleukin-6 (IL-6) of at least about 80 pg/ml.

In certain embodiments, the subject is administered a NELL1 polypeptide or a nucleic acid molecule encoding the same after testing positive for COVID-19 or when exhibiting symptoms of COVID-19. In certain embodiments, a NELL1 polypeptide or nucleic acid molecule encoding the same is administered via inhalation or systemically.

In some embodiments, the subject has pneumonia. In certain embodiments, the subject as ALI or ARDS. In particular embodiments, the subject is on supplementary oxygen or artificial ventilation.

In certain embodiments, the NELL 1 polypeptide has an amino acid sequence having at least 80% sequence identity to the amino acid sequence set forth as SEQ ID NO: 2, 4, 6, 10, or 12. In some of these embodiments, the NELL1 polypeptide is the polypeptide of SEQ ID NO: 2, 4, 6, 10, 12, 17, or 18. In some of those embodiments wherein the method comprises administering a nucleic acid molecule encoding a NELL1 polypeptide, the nucleic acid molecule is comprised within an expression vector and operably linked to a promoter. The subject can be a mammal, such as a human.

### BRIEF DESCRIPTION OF THE DRAWINGS

FIG. 1 shows the delivery of NELL1 to a transgenic mouse model of SARS-Co-V-2 infection. 1.25 mg/kg (A) or 2.5 mg/kg (B) NELL1 protein was administered to tg-mice hACE2r by retro-orbital injection on days 1 and 3 post-infection with SARS-Co-V-2. FIG. 1A demonstrates that the lower dose (1.25 mg/kg BW, n=5) of NELL1 protein induces substantial increase in body weight compared to uninfected control mice. The higher dose (2.5 mg/kg BW, n=5) of NELL1 was not as effective. FIG. 1B provides the corresponding Kaplan-Meier survival plot indicating a 40% survival with the lower dose of NELL1 and a 20% survival with the higher dose of NELL1.

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes and are not to be construed as limiting the subject matter described. Finally, for the purposes of the instant disclosure all identifying sequence Accession numbers may be found in the NCBI Reference Sequence (RefSeq) database and/or the NCBI GenBank archival sequence database unless otherwise noted.

### I. Viruses

Viruses are infectious agents that depend upon their hosts for replication. In certain embodiments, the virally-induced tissue damage and/or inflammation that is being treated with a NELL1 polypeptide or nucleic acid molecule encoding the same is a virus that infects animals. In some of these embodiments, the virus is one that infects mammals. In particular embodiments, the virus is one that infects humans.

Viral proteins and/or the virus itself can stimulate an inflammatory cascade which can cause damage to its host. In some embodiments, the virally-induced tissue damage and/or inflammation that is being treated with a NELL1 polypeptide or a nucleic acid encoding the same is caused by a virus that can cause a cytokine storm in its host. A cytokine storm causes cytokine storm syndrome or cytokine release syndrome (CRS) in the subject suffering a viral infection. CRS is a severe, acute systemic inflammatory response that occurs when large numbers of white blood cells are activated and release inflammatory cytokines, which in turn activate yet more white blood cells in a positive feedback loop of pathogenic inflammation. A cytokine storm can lead to systemic hyper-inflammation, hypotensive shock, and multi-organ failure. In some embodiments, the cytokine storm involves elevated levels (when compared to a control subject not infected by a virus) of at least one of the following cytokines: interleukin-6 (IL-6), IL-1, IL-1ra, IL-2R, IL-2ra, IL-10, IL-18, hepatocyte growth factor (HGF), interferon-gamma (IFN-γ), tumor necrosis factor-alpha (TNF-α), CCL-2/MCP-1, CXCL-10/interferon gamma induced protein 10 (IP-10), monocyte chemotactic protein-3 (MCP-3), macrophage inflammatory protein 1 alpha (MIG-1a), macrophage colony stimulating factor (M-CSF), granulocyte colony-stimulating factor (G-CSF), and cutaneous T-cell-attracting chemokine (CTACK).

Viruses known to cause CRS in some patients include but are not limited to influenza, SARS-CoV, MERS-CoV, and SARS-CoV-2. Infection by SARS-CoV-2, for example, can lead to a cytokine storm and systemic hyperinflammation resulting in inflammatory lymphocytic and monocytic infiltration of the lung and the heart, causing ARDS and cardiac failure. Patients with COVID-19 and ARDS have classical biomarkers of cytokine release syndrome including elevated C-reactive protein (CRP), lactate dehydrogenase (LDH), IL-6, and ferritin (Zhang C et al. 2020 International Journal of Antimicrobial Agents on the world wide web at doi.org/10.1016/j.ijantimicag.2020.105954).

Replication of the virus within a cell can weaken or even eventually kill the cell by usurping the cellular machinery for its own replication, thus causing tissue damage.

Some viruses are lytic, lysing the host cell in order to release the virus. The process of usurping the host's cellular machinery in order to replicate and the subsequent lysis of the cells also causes tissue damage in the host. Thus, in some embodiments, the virally-induced tissue damage and/or inflammation that is being treated with a NELL1 polypeptide or a nucleic acid encoding the same is caused by a lytic virus.

In other embodiments, the virus is an enveloped virus. Viral envelopes comprise the outer layer of the virus and the lipid bilayer envelope is often derived from portions of the host cell's outer cell membrane or nuclear, endoplasmic reticulum or endosomal membranes. Enveloped viruses possess great adaptability and can change in a short time period in order to evade the immune system. Non-limiting examples of enveloped viruses include herpesvirus, poxviruses, hepadnaviruses, Asfarviridae, flavivirus, alphavirus, togavirus, coronavirus, Hepatitis D, orthomyxovirus, paramyxovirus, rhabdovirus, bunyavirus, filovirus, influenza viruses, and retroviruses.

In some embodiments, the virus is an enveloped virus that replicates within its host cell, followed by budding off of the viral particles. The viral envelopes of these viruses thus comprise portions of the host cell plasma membrane (phospholipids and proteins), as well as viral proteins. This might help these viruses avoid the host immune system. Non-limiting examples of enveloped viruses that bud from their host include retroviruses, paramyxoviruses, influenza viruses, orthomyxoviruses, arenaviruses, filoviruses, human immunodeficiency virus type-1 (HIV-1), Ebola virus, and Rous sarcoma virus.

In still other embodiments, the virus is an enveloped virus that replicates within its host cell, followed by release via exocytosis of viral particles. These viruses comprise portions of the host cell endoplasmic reticulum, endosomal or nuclear membranes. Non-limiting examples of such viruses include coronaviruses, varicella-zoster virus, rotavirus, vaccinia virus, Herpes simplex virus, Hepatitis B virus, and Dengue virus. Some coronaviruses, which are enveloped viruses that are released from host cells via exocytosis, express spike (S) proteins on the host cell surface where these proteins mediate cell-cell fusion between infected cells and adjacent uninfected cells. This leads to giant, multinucleated cells, allowing the virus to spread within an infected host while avoiding detection and neutralization by virus-specific antibodies. Thus, in some embodiments, the virus is an enveloped virus that is released from its host cell via exocytosis and generates multinucleated cells to mediate cell-to-cell infection.

Respiratory viruses are viruses that infect the upper and/or lower respiratory tract. Morbidity may result directly from viral infection or may be indirect, due to exacerbation of underlying cardiopulmonary conditions. Non-limiting examples of respiratory viruses include respiratory syncytial virus (RSV), influenza viruses (including influenza A viruses such as H1N1 and H3N2, and influenza B viruses), rhinoviruses, adenovirus, human metapneumovirus (hMPV), parainfluenza virus, and coronaviruses. In some embodiments, the virally-induced tissue damage and/or inflammation that is being treated with a NELL1 polypeptide or a nucleic acid encoding the same is caused by a respiratory virus. In other embodiments, the virus that causes tissue damage and/or inflammation is a virus that infects the upper and/or lower respiratory tract and the heart and/or vasculature.

In some embodiments, the virally-induced tissue damage and/or inflammation that is being treated with a NELL1 polypeptide or a nucleic acid encoding the same is caused by a coronavirus. Coronaviruses are viruses in the Coronaviridae family that are enveloped, positive-sense single-stranded RNA viruses. On the surface of coronaviruses are club-shaped spike projections comprised of the spike protein. Coronaviruses utilize the spike proteins for attachment to host cells. In some embodiments, the virally-induced tissue damage and/or inflammation that is being treated with a NELL1 polypeptide or a nucleic acid encoding the same is caused by a virus belonging to the alpha group of the Coronaviridae family. In some embodiments, the virally-induced tissue damage and/or inflammation that is being treated with a NELL1 polypeptide or a nucleic acid encoding the same is caused by a virus belonging to the beta group of the Coronaviridae family. In some embodiments, the virally-induced tissue damage and/or inflammation that is being treated with a NELL1 polypeptide or a nucleic acid encoding the same is caused by a virus belonging to the gamma group of the Coronaviridae family. In some embodiments, the virally-induced tissue damage and/or inflammation that is being treated with a NELL1 polypeptide or a nucleic acid encoding the same is caused by a virus belonging to the delta group of the Coronaviridae family. SARS-CoV-2 shares a highly similar gene sequence and behavior pattern with SARS-CoV (Chan et al., Emerg Microbes Infect. 2020; 9(1):221-236). Both SARS-CoV-2 and SARS-CoV are in the coronavirus family, β-coronavirus genera, lineage B (Chan et al., Id.). In certain embodiments, the virally-induced tissue damage and/or inflammation that is being treated with a NELL1 polypeptide or a nucleic acid encoding the same is caused by a β coronavirus, lineage B (i.e., SARS virus). In particular embodiments, the coronavirus is SARS-CoV-2.

The current raging global health crisis caused by a novel coronavirus called severe acute respiratory syndrome coronavirus 2 (SARS-CoV-2) leads to coronavirus disease 2019 (COVID-19) (Cascella M et al. 2020; Shereen M A et al. 2020; Velavan T P and Meyer C G 2020 Tropical Medicine and International Health 25(3):278-280, doi:10.1111/tmi.13383). SARS-CoV-2 is a novel pathogen in humans that has only recently changed its transmission from animal-to-human into human-to-human (Sheeran M A et al. 2020; Cascella M et al. 2020). Therefore, the current pandemic (2019-present) displays the first glimpses into the nature of SARS-CoV-2 infection and its adverse biological effects on various human tissues such as the respiratory, cardiovascular and digestive systems (Cascella M et al. 2020; Singhal T 2020 The Indian Journal of Pediatrics 87(4):281-286; Tian S et al. 2020 Journal Thoracic Oncology https://doi.org/10.1016/j.jtho.2020.02010; Zheng Y Y et al. 2020 Nature Reviews Cardiology on the world wide web at doi.org/10.1038/s41569-020-0360-5). In particular embodiments, the virally-induced tissue damage and/or inflammation that is being treated with a NELL1 polypeptide or a nucleic acid encoding the same is caused by SARS-CoV-2. SARS-CoV-2 is a beta-coronavirus. SARS-CoV-2 virus can refer to the original virus discovered in Wuhan, China in 2019 (Xu et al., Genomics Proteomics Bioinformatics. 2003 August; 1(3): 226-235; herein incorporated by reference in its entirety), the genome sequence of which is set forth as NCBI Reference Sequence NC_045512.2 (herein incorporated by reference in its entirety) or a variant thereof, including the six types of the strain (types I to VI) described by Yang et al. (2020) Proc Natl Acad Sci USA 117(48):30679-30686, which is herein incorporated by reference in its entirety, 20I/501Y.V1, VOC 202012/01 or B.1.1.7 variant, the 20H/501Y.V2 or B.1.351 variant, or the P1 variant. Non-limiting examples of SARS-CoV-2 genome sequences include GenBank Accession No. MN908947.3, NCBI Reference Sequence NC_045512.2, and Global Initiative on Sharing Avian Influenza Data (GISAID) Accession IDs: EPI_ISL_404227, EPI_ISL_404228, EPI_ISL_402132, EPI_ISL_402127, EPI_ISL_402128, EPI_ISL_402129, EPI_ISL_402130, EPI_ISL_402124, EPI_ISL_403963, EPI_ISL_403962, EPI_ISL_402120, EPI_ISL_402119, EPI_ISL_402121, EPI_ISL_402123, EPI_ISL_402125, EPI_ISL_403931, EPI_ISL_403928, EPI_ISL_403930, EPI_ISL_403929, EPI_ISL_403937, EPI_ISL_403936, EPI_ISL_403935, EPI_ISL_403934, EPI_ISL_403933, EPI_ISL_403932, EPI_ISL_404895, EPI_ISL_404253, and EPI_ISL_405839.

SARS-CoV, HCoV-NL63, and the novel SARS-CoV-2 utilize angiotensin-converting enzyme 2 (ACE2) as their receptor and entry point via receptor-mediated endocytosis. ACE2 also functions to protect the lungs from virus-induced injury by increasing the production of vasodilator angiotensin 1-7, and therefore viral binding to this receptor deregulates a lung protective pathway. ACE2 is a zinc containing metalloenzyme expressed on the surface of various cell types (human ACE2 precursor proteins are set forth as NCBI Genbank Accession Nos. NP_068576.1 and NP_001358344.1). Thus, in particular embodiments, the virally-induced tissue damage and/or inflammation that is being treated with a NELL1 polypeptide or a nucleic acid encoding the same is caused by a virus (e.g., coronavirus) that attaches to ACE2 and utilizes ACE2 for entry into the host cell.

SARS-CoV and SARS-CoV-2 primarily infect epithelial cells within the lung. In fact, 83% of ACE2-expressing cells are alveolar epithelial type II (ATII) cells, one of the two types of alveolar epithelial cells in the lung (Zhang et al. (2020) Intensive Care Med 46:586-590). Although the main target is lung tissue, the virus also attacks other linings of the respiratory system, including the oral mucosa (Xu et al. (2020) International Journal of Oral Science 12:8), and other organs such as the esophagus and GI tract. In addition to the lung epithelium, ACE2 is also expressed in the heart, kidney, endothelium, intestine, and skeletal muscle, all tissues that co-express NELL1.

In some embodiments, the virally-induced tissue damage and/or inflammation that is being treated with a NELL1 polypeptide or a nucleic acid encoding the same is caused by a virus belonging to the alpha group of the Orthomyxoviridae family. According to some embodiments, the virus belonging to the Orthomyxoviridae family is an influenza virus. According to some embodiments, the virus belonging to the Orthomyxoviridae family is from the genus Influenza virus A. According to some embodiments, the virus belonging to the Orthomyxoviridae family is from the genus Influenza virus B. According to some embodiments, the virus belonging to the Orthomyxoviridae family is from the genus Influenza virus C. According to some embodiments, the virus belonging to the Orthomyxoviridae family is from the genus Influenza virus D.

Influenza type A (IAV) viruses pose one of the world's greatest health and economic burdens. Some 5,000 to 50,000 yearly deaths by seasonal flu occur in the USA alone, and 300,000 to 600,000 worldwide. Across the globe, 5-10% adults and 20%-30% children are annually infected by seasonal flu, with 90% fatalities for those with immature or immunocompromised system (children younger than 5, elderly older than 65, pregnant women with an inherited or acquired immunodeficiency undergoing chemotherapy, or with chronic medical conditions like COPD or asthma). As the pandemic of 1918 Spanish flu resulted in a devastating 50 million deaths across Europe and around the globe.

IAVs are enveloped orthomyxovirions with a segmented RNA genome of negative polarity. The virion encodes 17 proteins including the newly identified NS3, M42, PA-N182, and PA-N155. Hemagglutinin (HA) and neuraminidase (NA) are the major viral envelope proteins. So far, 131 combinations between eight different HA and eleven NA proteins have been identified in humans and animals. The structural integrity of the viral proteins is continuously compromised by high mutagenesis due to the lack of proofreading by the viral RNA polymerase. Such unpredictable mutations are responsible for HA and NA antigenic drifts that increases the risk of new epidemic or pandemic outbreaks. The most common IAV heterosubtypes circulating in humans are H1N1 and H3N2.

In some embodiments, the virally-induced tissue damage and/or inflammation that is being treated with a NELL1 polypeptide or a nucleic acid encoding the same is caused by a virus belonging to the alpha group of the Paramyxoviridae family. According to some embodiments, the virus belonging to the Paramyxoviridae family is from the genus Paramyxovirus or Pneumovirus. According to some embodiments, the virus belonging to the Paramyxoviridae family, genus Paramyxovirus is parainfluenza virus (NV). According to some embodiments, the virus belonging to the Paramyxoviridae family, genus Pneumovirus is respiratory syncytial virus (RSV).

### II. NELL1

The neural epidermal growth-factor-like (nel) gene was first detected in neural tissue from an embryonic chicken cDNA library, and its human ortholog neural epidermal growth-factor-like 1 (NEL-like 1, NELL1) was discovered later in B-cells. Studies have reported the presence of NELL1 in various fetal and adult organs, including, but not limited to, skeletal and cardiac muscle, skin, the brain, kidneys, colon, thymus, lung, and small intestine.

The human NELL1 gene encodes an 810-amino acid polypeptide. Generally, the arrangement of the functional domains of the NELL1 protein bears resemblance to thrombospondin-1 (THBS1) and consists of a thrombospondin N-terminal domain (TSPN) and several von Willebrand factor, type C (VWC), and epidermal growth-factor (EGF) domains. A domain is a region of a protein with a characteristic primary structure and function.

Additional studies have shown that there are at least two human NELL1 transcript variants encoding different isoforms. In humans, the nel-like 1 isoform 1 precursor transcript variant (set forth in SEQ ID NO: 1) represents the longer transcript (set forth in GenBank Acc. No. NM_006157) and encodes the longer isoform 1 (set forth in SEQ ID NO: 2).

The conserved domains of NELL1 reside in seven regions of the isoform 1 peptide and include: (1) a TSPN domain/Laminin G superfamily domain; (2) a VWC domain; (3) four EGF-like domains; and (4) a VWC domain. NELL1 also comprises a secretion signal peptide domain (amino acid residues 1-16 of SEQ ID NO: 2) that is generally involved in transport of the protein to cell organelles where it is processed for secretion outside the cell.

The first conserved domain region comprises amino acids (amino acids 29 to 213 of SEQ ID NO: 2) that are most similar to a thrombospondin N-terminal-like domain. Thrombospondins are a family of related, adhesive glycoproteins, which are synthesized, secreted and incorporated into the (ECM) of a variety of cells, including alpha granules of platelets following thrombin activation and endothelial cells. They interact with a number of blood coagulation factors and anticoagulant factors, and are involved in cell adhesion, platelet aggregation, cell proliferation, angiogenesis, tumor metastasis, vascular smooth muscle growth and tissue repair. The first conserved domain also comprises amino acids (amino acids 82 to 206; amino acids 98 to 209 of SEQ ID NO: 2) that are similar to a Laminin G-like domain. Laminin G-like (LamG) domains usually are Ca′ mediated receptors that can have binding sites for steroids, β1-integrins, heparin, sulfatides, fibulin-1, and α-dystroglycans. Proteins that contain LamG domains serve a variety of purposes, including signal transduction via cell-surface steroid receptors, adhesion, migration and differentiation through mediation of cell adhesion molecules.

Studies show that NELL1 signaling involves an integrin-related molecule and tyrosine kinases that are triggered by NELL1 binding to a NELL1 specific receptor and a subsequent formation of an extracellular complex. As thus far understood, in human NELL1 (hNELL1), the laminin G domain comprises about 128 amino acid residues that show a high degree of similarity to the laminin G domain of extracellular matrix (ECM) proteins; such as human laminin α3 chain (hLAMA3), mouse laminin α3 chain (mLAMA3), human collagen 11 α3 chain (hCOLA1), and human thrombospondin-1 (hTSP1). This complex facilitates either activation of tyrosine kinases, inactivation of tyrosine phosphatases, or intracellular recruitment of tyrosine-phosphorylated proteins. The ligand bound integrin (cell surface receptors that interact with ECM proteins such as, for example, laminin 5, fibronectin, vitronectin, TSP1/2) transduces the signals through activation of the focal adhesion kinase (FAK) followed by indirect activation of the Ras-MAPK cascade, and then leads to osteogenic differentiation through Runx2; the laminin G domain is believed to play a role in the interaction between integrins and a 67 kDa laminin receptor (Shen et al. (2012) J Cell Biochem 113:3620-3628).

The second conserved domain (amino acids 273 to 331 of SEQ ID NO: 2) and seventh conserved domain (amino acids 701 to 749 of SEQ ID NO: 2) are similar to von Willebrand factor type C (VWC) domains, also known as chordin-like repeats. An additional VWC domain is also found from amino acid residues 634 to 686 of SEQ ID NO: 2. VWC domains occur in numerous proteins of diverse functions and have been associated with facilitating protein oligomerization.

The third conserved domain (amino acids 434 to 466 of SEQ ID NO: 2), fourth conserved domain (amino acids 478 to 512 of SEQ ID NO: 2), fifth conserved domain (amino acids 549 to 586 of SEQ ID NO: 2), and sixth conserved domain (amino acids 596 to 627 of SEQ ID NO: 2) are similar to a calcium-binding EGF-like domain. Calcium-binding EGF-like domains are present in a large number of membrane-bound and extracellular (mostly animal) proteins. Many of these proteins require calcium for their biological function. Calcium-binding sites have been found to be located at the N-terminus of particular EGF-like domains, suggesting calcium-binding may be crucial for numerous protein-protein interactions. Six conserved core cysteines form three disulfide bridges as in non-calcium-binding EGF domains whose structures are very similar. The calcium-binding EGF-like domains of NELL1 bind protein kinase C beta, which is typically involved in cell signaling pathways in growth and differentiation.

The nel-like 1 isoform 2 precursor transcript variant (set forth in GenBank Acc. No. NM_201551 and SEQ ID NO: 3) lacks an alternate in-frame exon compared to variant 1. The resulting isoform 2 (set forth in SEQ ID NO: 4), which has the same N- and C-termini as isoform 1 but is shorter compared to isoform 1, has six conserved regions including a TSPN domain/LamG superfamily domain (amino acids 29 to 213 of SEQ ID NO: 4); VWC domains (amino acids 273 to 331 of SEQ ID NO: 4; amino acids 654 to 702 of SEQ ID NO: 4); and calcium-binding EGF-like domains (amino acids 478 to 512 of SEQ ID NO: 4; amino acids 434 to 466 of SEQ ID NO: 4; amino acids 549 to 580 of SEQ ID NO: 4).

NELL1 and its orthologs are found across several species including Homo sapiens (man), Bos taurus (cow; the nucleic acid sequence of which is set forth in GenBank Acc. No. XM_002699102 and the amino acid sequence is set forth in SEQ ID NO: 19), Equus caballus (horse; the nucleic acid sequence of isoforms 1 and 2 are set forth in GenBank Acc. Nos. XM_001504986 and XM_001504987, respectively, and in SEQ ID NO: 5 and 7, respectively; the amino acid sequences are set forth in SEQ ID NO: 6 and 8, respectively), Macaca mulatta (rhesus monkey; the nucleic acid sequence of isoforms 1, 2, 3, and 4 are set forth in GenBank Acc. Nos. XM_002799606, XM_001092428, XM_001092540, and XM_001092655, respectively), Mus musculus (mouse; the nucleic acid sequence of which is set forth in GenBank Acc. No. NM_001037906 and in SEQ ID NO: 9; the amino acid sequence of which is set forth in SEQ ID NO: 10), Rattus norvegicus (rat; the nucleic acid sequence of which is set forth in GenBank Acc. No. NM_031069 and in SEQ ID NO: 11; the amino acid sequence of which is set forth in SEQ ID NO: 12), Pan troglodytes (chimpanzee; the nucleic acid sequence of which is set forth in GenBank Acc. No. XM_508331.2), Felis catus (cat; the amino acid sequences of isoform 1 and 2 are set forth in GenBank Acc. Nos. XP 003993117.1 and XP 003993118.1, and SEQ ID NOs: 13 and 14, respectively, Canis lapis familiaris (dog; the amino acid sequence is set forth in GenBank Acc. No. XP 534090 and SEQ ID NO: 15), and Ovis aries (sheep; the amino acid sequence is set forth in GenBank Acc. No. XP_004019490 and SEQ ID NO: 16).

NELL1 is an extracellular protein that is abundant during mammalian fetal development and mediates pathways encompassing many signaling and structural proteins, that are essential for promoting and balancing tissue growth and maturation (Matsuhashi S et al. 1995 Dev Dyn 203:2012-22; Ting K et al. 1999 J Bone Miner Res 14:80-9; Zhang X et al. 2002 J Clin Invest 110:861-870; Desai J et al. 2006 Hum Mol Genet 15(8):1329-1341; Li C et al. 2017 Am J Pathol 187(5):963-972, doi:10.1016/j.ajpath.2016.12.026; Li C et al. 2018 Am J Pathol 188(2):392-403, doi:10.1016/j.apath.2017.09.020). A rapidly increasing body of published studies on in vitro and in vivo (small and large animals) models have demonstrated NELL1's ability to restore and regenerate functional tissue after acute injury in bone (Lu S S et al. 2007 Spine J 7(1):50-60; Aghaloo T et al. 2007 Mol Ther 15(10):1872-1880; Xue J et al. 2011 Bone 48(3):485-95; Aghaloo T et al. 2006 Am J of Path 169(3):903-915; Cowan C M et al. 2006 Bone 38:48-58; Tanjaya J et al. 2018 The American Journal of Pathology 188(3):715-727; Li W et al. 2011 Plast Reconstr Surg 127(2):580-587; Siu R K et al. 2011 Tissue Eng Part A 17(7-8):1123-1135), cartilage (Lee Metal. 2010 Tissue Eng Part A 16(5):1791-1800; Siu R K et al. 2012 Tissue Eng Part A 18(3-4):252-61, doi: 10.1089/ten.TEA.2011.0142; Pakvasa M et al. 2017 Genes and Diseases 4:127-137; Li C et al. 2018 The American Journal of Pathology 188(2):392-403; Li C et al. 2020 Biomaterials 226:119541), skin and muscle (Mitchell D et al. 2012 Journal of the American Academy of Dermatology 66(4): Supplement 1, Page AB3; Turner N et al. 2013 Cells, Tissues and Organs 198(4):249-265; Chen H. et al. 2018 Brazilian J of Med and Biol Res 51(6):ee6997). Moreover, human genetic studies and small animal models have established the role of NELL1 in maintaining the balance of cell growth vs. differentiation and tissue formation vs. breakdown, especially in organs where rapid continual breakdown and renewal are necessary to maintain function—bone, epithelial linings of the esophagus, and gastrointestinal tract (James A W et al. 2015 Nature Communications 6:7362, doi:10.1038/ncomms8362; Jin Z et al. 2007 Oncogene doi:10.1038/sj.onc.1210461; Mori Y et al. 2006 Gastroenterology 131:797-808; Nakamura R et al. 2014 J. Biol. Chem doi:10.1074/jbc.M113.507020). During early development, NELL1 regulates the production of many components of the extracellular matrix (ECM) which collectively serve as an architectural framework and communication highway to mediate new tissue formation.

While not being bound by any particular theory or mechanism of action, it is believed that NELL1 treats tissue damage (e.g., lung, heart, vasculature, skeletal muscle) in viral infections, such as SARS-CoV-2, by promoting biological pathways that:
a) reduce inflammation by controlling levels of major pro-inflammatory factors (e.g., IL-1/IL-6/TNF-alpha) that are major players in the cytokine storm which overwhelms the body during the early phase of infection (Li C et al. 2020 Biomaterials 226:119541; Mitchell D et al. 2012 Journal of the American Academy of Dermatology 66(4): Supplement 1, page AB3; Shen J. et al. 2013 Tissue Engineering Part A 19(21-22):2390-2401; Chen H et al. 2018 Brazilian J of Med and Biol Res 51(6): ee6997);
b) increase tissue survival under hypoxia which provides time and opportunity to initiate and sustain repair mechanisms;
c) promote angiogenesis;
d) enhance cell division and differentiation of tissues to replace damaged tissues (Jin Z et al. 2007 Oncogene 1-7; Mori Y et al. 2006 Gastroenterology 131:797-808; Franke A et al. 2007 PLoS One 2(8):e691); and/or
e) recruitment of stem cells to injury site for tissue formation and blood vessel formation (Pakvasa M et al. 2017 Genes & Diseases 4:127-137; Zhang X et al. 2011 Tissue Engineering Part A 17(19-20); James A W et al. 2017 JCI Insight on the world wide web at doi.org/10.1172/jci.insight.92573; Askarinam A et al. 2013 Tissue Engineering Part A 19(11-12)1386-1397).

The presently disclosed methods utilize a NELL1 polypeptide or a nucleic acid molecule encoding the same to treat virally-induced tissue damage and inflammation. As used herein and in the claims, a “NELL1 polypeptide” refers to a naturally occurring NELL1 polypeptide of any species, as well as variants and fragments of such naturally occurring polypeptides as described herein.

A peptide, polypeptide, or protein is a sequence of subunit amino acids, amino acid analogs, or peptidomimetics. A peptidomimetic is a small protein-like chain designed to mimic a peptide. A peptidomimetic typically arises from modification of an existing peptide in order to alter the molecule's properties.

A peptide, polypeptide or protein can also be amino acid polymers in which one or more amino acid residue is an artificial chemical analogue of a corresponding naturally occurring amino acid, as well as to naturally-occurring amino acid polymers. A polypeptide, peptide or protein is inclusive of modifications including, but not limited to, glycosylation, lipid attachment, sulfation, gamma-carboxylation of glutamic acid residues, hydroxylation, phosphorylation, and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing events and events brought about by human manipulation which do not occur naturally. Circular, branched and branched circular polypeptides may be synthesized by non-translation natural processes and by entirely synthetic methods, as well.

NELL1 has regenerative properties. The regeneration of tissue refers to the process of renewal and growth of cells and extracellular matrix components within a particular tissue that results in the production of tissue that has a cellular component and architecture that allows for the normal functions of the particular tissue type. A NELL1 peptide, NELL1 polypeptide, or NELL1 protein is a naturally-occurring NELL1 protein, or a variant or fragment thereof that retains the ability to regenerate or maintain healthy tissue. In some embodiments, the NELL1 polypeptide exhibits any one of the activities selected from the group consisting of: stimulation of ECM production (e.g., through the upregulation of at least one of tenascins, proteoglycans, elastin, glycosaminoglycans, including epidermal hyaluronic acid, and collagens), reduction in the levels of inflammatory mediators (e.g., IL-1β and IL-8), and reduction in the levels of matrix metalloproteinases (e.g., MMP1).

In other embodiments, the NELL1 polypeptide can also exhibit at least one of the activities selected from the group consisting of: binding to PKC-beta, stimulation of differentiation of a precursor cell (e.g., mesenchymal stem cell, immature heart cells, epithelial precursor) to maturity, and stimulation of angiogenesis. To determine whether a polypeptide exhibits any one of these activities, any method known in the art useful for measuring these activities can be used.

Suitable assays for determining if a given polypeptide can stimulate ECM production and reduce the levels of inflammatory mediators or MMPs include assays that measure transcript levels (e.g., quantitative polymerase chain reaction) or levels of the protein (e.g., enzyme-linked immunoassay) directly or indirectly (by measuring the activity of the protein), including those that are described elsewhere herein.

Suitable assays for assessing the binding of NELL1 to PKC beta is described in e.g., Kuroda et al. (1999) Biochem Biophys Res Comm 265:752-757. For example, protein-protein interactions can be analyzed by using the yeast two-hybrid system. Briefly, a NELL1 polypeptide can be fused with GAL4 activating domain and the regulatory domain of PKC can be fused with the GAL4 DNA-binding domain.

The NELL1 polypeptide may be a naturally-occurring (i.e., wild-type) NELL1 protein or an active variant or fragment thereof. Naturally refers to as found in nature; wild-type; innately or inherently. A naturally-occurring NELL1 polypeptide may be purified from a natural source or may be a polypeptide that has been recombinantly or synthetically produced that has the same amino acid sequence as a NELL1 polypeptide found in nature.

A polynucleotide can be a singular nucleic acid, as well as plural nucleic acids, and refers to a nucleic acid molecule or construct, e.g., messenger RNA (mRNA), complementary DNA (cDNA), or plasmid DNA (pDNA). A polynucleotide (e.g., nucleic acid molecule) can be single-stranded or double-stranded, linear or circular and can be comprised of DNA, RNA, or a combination thereof. A polynucleotide (e.g., nucleic acid molecule) can comprise a conventional phosphodiester bond or a non-conventional bond (e.g., an amide bond, such as found in peptide nucleic acids (PNA)). A nucleic acid can be any one or more nucleic acid segments, e.g., DNA or RNA fragments, present in a polynucleotide. The polynucleotide (e.g., nucleic acid molecule) can contain modified nucleic acids, such as phosphorothioate, phosphate, ring atom modified derivatives, and the like. The polynucleotide (e.g., nucleic acid molecule) can be a naturally occurring polynucleotide (i.e., one existing in nature without human intervention), a recombinant polynucleotide (i.e., one existing with human intervention), or a synthetically derived polynucleotide.

An isolated material can refer to a nucleic acid, peptide, polypeptide, or protein, which is: (1) substantially or essentially free from components that normally accompany or interact with it as found in its naturally occurring environment. Substantially free or essentially free refer to considerably or significantly free of, or more than about 95% free of, or more than about 99% free of. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state. For example, a naturally occurring nucleic acid becomes an isolated nucleic acid if it is altered, or if it is transcribed from DNA that has been altered, by means of human intervention performed within the cell from which it originates. See, for example, Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (for example, a promoter) becomes isolated if it is introduced by non-naturally occurring means to a locus of the genome not native to that nucleic acid.

Fragments and variants of native (i.e., naturally-occurring) NELL polypeptides can be employed in the various methods and compositions of the invention. A fragment is intended a portion of a polynucleotide or a portion of a polypeptide. Fragments of a polynucleotide may encode polypeptide fragments that retain the biological activity of the native polypeptide. A fragment of a polynucleotide that encodes a biologically active portion of a NELL1 polypeptide will encode at least 15, 25, 30, 50, 100, 150, 200, 250, 300, 350, 400, 450, 500, 550, 600, 650, 700, 750, or 800 contiguous amino acids, or up to the total number of amino acids present in a full-length NELL1 polypeptide. In certain embodiments, the NELL1 fragment is 610 amino acids in length.

A fragment of a native NELL1 polypeptide can be prepared by isolating a portion of a polynucleotide encoding the portion of the NELL1 polypeptide and expressing the encoded portion of the polypeptide (e.g., by recombinant expression in vitro). Polynucleotides that encode fragments of a NELL1 polypeptide can comprise nucleotide sequences comprising at least 15, 20, 50, 75, 100, 150, 200, 250, 300, 350, 400, 450, 500, 550, 600, 650, 700, 800, 900, 1000, 1100, 1200, 1300, 1400, 1500, 1600, 1700, 1800, 1900, 2000, 2100, 2200, 2300, or 2400 contiguous nucleotides, or up to the number of nucleotides present in a full-length NELL1 nucleotide sequence. In some embodiments, the fragment lacks the first amino acid residue, or the first 2, 3, 4, 5, 6, 7, 8, 9, 10, 20, 21, 22, 23, 24, 25, 26, 27, 28, 29, 30, 35, 40, or 45 amino acid residues from the amino terminal end of the NELL1 protein. In some embodiments, the fragment lacks the last 5, 10, 15, 20, 25, 30, 35, 40, 45, 50, 55, 60, 65, 70, 75, 80, 85, 90, 95, 100, 110, 115, 120, 125, 130, 135, 140, 145, 150, 155, 160, 165, 170, 175, 180, 185, 190, 195, 200, 205, 210, 220, 230, 240, 250, 260 or more amino acid residues. In certain embodiments, the fragment of a NELL1 protein lacks the most carboxy-terminal 179 amino acid residues from the end of the protein. In other embodiments, the NELL1 protein fragment lacks the first two amino acid residues from the amino terminal end and the last 179 amino acid residues from the carboxy terminal end of the protein. In some embodiments, the NELL1 protein fragment has 610 amino acid residues.

Removal of 179 amino acid residues from the carboxy-terminus of the Equus caballus NELL1 isoform 1 protein unexpectedly provided a higher yield and easier purification during manufacture of the protein (U.S. Patent Application Publication No. 2018/0057550). Without being bound by any theory or mechanism of action, it is believed that the removal of the carboxy-terminal domains led to decreased formation of aggregates of the protein. Although NELL1 protein naturally oligomerizes into trimers, which are functional, aggregates of NELL1 protein refer to large, higher-ordered macromolecular complexes that prevent or reduce the function of the protein or make the protein products difficult to extract and purify. The NELL1 protein lacking the C-terminal 179 amino acid residues is also unexpectedly more efficacious than full-length NELL1 protein in horse body wound healing studies and fibroblast wound scratch assays. Thus, in specific embodiments, the NELL1 protein fragment lacks the last 179 amino acid residues from the carboxy terminus. In some of these embodiments, the NELL1 protein fragment also lacks the first two amino acid residues from the amino terminus. The sequence of this horse NELL1 fragment is set forth in SEQ ID NO: 18. In other embodiments, the NELL1 protein fragment lacks the first 21 amino acid residues from the amino terminus and the last 179 amino acid residues from the carboxy terminus. The sequence of this human NELL1 fragment is set forth in SEQ ID NO: 17, also referred to herein as NV1. In certain embodiments, the NELL1 protein fragment lacks at least one of the two carboxy-terminal VWC domains (located at amino acid residues 634-686 and 701-749 of SEQ ID NO: 2). In some of these embodiments, the NELL1 protein fragment lacks both of these carboxy-terminal VWC domains.

In those embodiments wherein a NELL1 protein fragment lacks at least one C-terminal VWC domain, the NELL1 protein fragment exhibits at least one of the following characteristics: enhanced efficacy in tissue regeneration and/or promotion of wound healing, enhanced prevention of tissue loss, easier purification, higher yield, less aggregate formation, and enhanced efficacy in fibroblast migration and/or proliferation, when compared to its respective full-length NELL1 protein. An easier purification includes a purification process whereby a single polypeptide species is substantially separated from other polypeptide species or a natural or synthetic milieu comprising the single polypeptide species and other polypeptide species that comprises fewer steps required for substantial separation or wherein the time required for at least one of the steps in the separation is reduced. An easier purification also refers to a purification process which results in a higher yield of the substantially purified or separated polypeptide species when compared to its respective full-length protein. The terms “substantially purified” or “substantially separated” when used in reference to a single polypeptide species refers to a level of purification whereby the single polypeptide species represents at least about 70% of a total population of polypeptide species within a sample, including but not limited to at least about 75%, 80%, 85%, 86%, 87%, 88%, 89%, 90%, 91%, 92%, 93%, 94%, 95%, 96%, 97%, 98%, 99%, or greater of a total population of polypeptide species within a sample. A yield of a protein product from a purification process refers to the overall concentration of the polypeptide within a solution. The higher the concentration of the polypeptide within the solution, the more yield is obtained. If a polypeptide is present within a solution at <0.1 μg/μl, the protein is considered difficult to produce and purify. Thus, in some embodiments, a NELL1 protein fragment that lacks at least one C-terminal VWC domain exhibits the ability to be purified using conventional purification means known in the art, such as those methods described elsewhere herein, to a concentration greater than 0.1 μg/μl. In some of these embodiments, a NELL1 protein fragment has the ability to be purified using conventional purification means known in the art to a concentration of about 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30 μg/μl, or greater. In certain embodiments, a NELL1 protein fragment lacking at least one C-terminal VWC domain exhibits both a higher yield and a greater purity as compared to its respective full-length NELL1 protein following a purification process.

Variant sequences have a high degree of sequence similarity. For polynucleotides, conservative variants include those sequences that, because of the degeneracy of the genetic code, encode the amino acid sequence of a NELL1 polypeptide. Variants such as these can be identified with the use of well-known molecular biology techniques, such as, for example, polymerase chain reaction (PCR) and hybridization techniques. Variant polynucleotides also include synthetically derived nucleotide sequences, such as those generated, for example, by using site-directed mutagenesis. In some embodiments, the variant polynucleotide still encodes a NELL1 polypeptide or a fragment thereof. Generally, variants of a particular polynucleotide will have at least about 60%, 65%, 70%, 75%, 80%, 85%, 90%, 91%, 92%, 93%, 94%, 95%, 96%, 97%, 98%, 99% or more sequence identity to that particular polynucleotide, when compared over the full length of the variant, as determined by sequence alignment programs and parameters described elsewhere herein.

Variants of a particular polynucleotide can also be evaluated by comparison of the percent sequence identity between the polypeptide encoded by a variant polynucleotide and the polypeptide encoded by the reference polynucleotide. Thus, variants include, for example, polynucleotides that encode a polypeptide with a given percent sequence identity to a native NELL1 polypeptide. Percent sequence identity between any two polypeptides can be calculated using sequence alignment programs and parameters described herein. Where any given pair of polynucleotides is evaluated by comparison of the percent sequence identity shared by the two polypeptides they encode, the percent sequence identity between the two encoded polypeptides is at least about 60%, 65%, 70%, 75%, 80%, 85%, 90%, 91%, 92%, 93%, 94%, 95%, 96%, 97%, 98%, 99% or more sequence identity.

A variant polypeptide is a polypeptide derived from the native polypeptide by deletion (so-called truncation) or addition of one or more amino acids to the N-terminal and/or C-terminal end of the native polypeptide; deletion or addition of one or more amino acids at one or more sites in the native polypeptide; or substitution of one or more amino acids at one or more sites in the native polypeptide. The activity of variant NELL1 polypeptides can be assessed using the methods disclosed herein to determine if the variant is biologically active. Such variants may result from, for example, genetic polymorphism or from human manipulation. Biologically active variants of a native NELL1 polypeptide will have at least about 60%, 65%, 70%, 75%, 80%, 85%, 90%, 91%, 92%, 93%, 94%, 95%, 96%, 97%, 98%, 99% or more sequence identity to the amino acid sequence for the native polypeptide, when compared over the full length of the variant, as determined by sequence alignment programs and parameters described elsewhere herein. A biologically active variant of a polypeptide may differ from that polypeptide by as few as 1-15 amino acid residues, as few as 1-10, such as 6-10, as few as 5, as few as 4, 3, 2, or even 1 amino acid residue.

Biologically active variants of the NELL1 fragments disclosed herein (i.e., those lacking at least one of the two VWC domains at the carboxy terminus of NELL1) are also contemplated herein and may have at least about 60%, 65%, 70%, 75%, 80%, 85%, 90%, 91%, 92%, 93%, 94%, 95%, 96%, 97%, 98%, 99% or more sequence identity to the amino acid sequence for the active NELL1 fragment (e.g., SEQ ID NO: 17 or 18).

Polypeptides may be altered in various ways including amino acid substitutions, deletions, truncations, and insertions. Methods for such manipulations are generally known in the art. For example, amino acid sequence variants of native NELL1 polypeptides can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the polypeptide of interest may be found in the model of Dayhoff et al. (1978) Atlas of Protein Sequence and Structure (Natl. Biomed. Res. Found., Washington, D.C.). Conservative substitutions, such as exchanging one amino acid with another having similar properties, may be preferable.

Generally, the mutations made in the polynucleotide encoding the variant NELL1 polypeptide should not place the sequence out of reading frame, and/or create complementary regions that could produce secondary mRNA structure. See, EP Patent Application Publication No. 75,444.

Variant NELL1 polynucleotides and polypeptides also encompass sequences and polypeptides derived from a mutagenic and recombinogenic procedure such as DNA shuffling. With such a procedure, one or more different NELL1 coding sequences can be manipulated to create peptides that can be evaluated to determine if it retains NELL1 activity. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

Variant NELL1 polynucleotides and polypeptides also encompass sequences and polypeptides derived from gene editing systems, such as CRISPR/Cas system.

Sequence identity in the context of two polynucleotides or polypeptide sequences makes reference to the residues in the two sequences that are the same when aligned for maximum correspondence over a specified comparison window. When percentage of sequence identity is used in reference to polypeptides it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have sequence similarity or similarity. Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

Percentage of sequence identity is the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3; % identity and % similarity for an amino acid sequence using GAP Weight of 8 and Length Weight of 2, and the BLOSUM62 scoring matrix; or any equivalent program thereof. An equivalent program is any sequence comparison program that, for any two sequences in question, generates an alignment having identical nucleotide or amino acid residue matches and an identical percent sequence identity when compared to the corresponding alignment generated by GAP Version 10.

The NELL1 polypeptide may be made synthetically, i.e. from individual amino acids, or semi-synthetically, i.e. from oligopeptide units or a combination of oligopeptide units and individual amino acids. Alternatively, the protein can be synthesized in a cell-free in vitro translation system, such as a wheat germ cell-free system (see, for example, Madin et al. (2000) Proc. Natl. Acad. Sci. U.S.A. 97(2):559-564; Sawasaki et al. (2000) Nucleic Acids Symp Ser 44:9-10; Sawasaki et al. (2002) Proc. Natl. Acad. Sci. U.S.A. 99(23):14652-14657; and Endo and Sawasaki (2003) Biotechnol. Adv. 21(8):695-713). Suitable methods for synthesizing proteins are described by Stuart and Young in “Solid Phase Peptide Synthesis,” Second Edition, Pierce Chemical Company (1984), Solid Phase Peptide Synthesis, Methods Enzymol., 289, Academic Press, Inc, New York (1997).

The NELL1 polypeptide may also be prepared by methods that are well known in the art. One such method includes isolating or synthesizing DNA encoding the NELL1 polypeptide, and producing the recombinant protein by expressing the DNA, optionally in a recombinant vector, in a suitable host cell. Suitable methods for synthesizing DNA are described by Caruthers et al. (1985) Science 230:281-285; and DNA Structure, Part A: Synthesis and Physical Analysis of DNA, Lilley, D. M. J. and Dahlberg, J. E. (Eds.), Methods Enzymol., 211, Academic Press, Inc., New York (1992).

In some embodiments of the presently disclosed methods, a nucleic acid molecule encoding a NELL1 polypeptide is administered to a subject in need thereof in order to treat virally-induced tissue damage and/or inflammation. As used herein, the terms “encoding” or “encoded” when used in the context of a specified nucleic acid mean that the nucleic acid comprises the requisite information to direct translation of the nucleotide sequence into a specified polypeptide.

In some embodiments of the presently disclosed methods, the NELL1 nucleic acid molecule is operably linked to at least one regulatory element. A regulatory element is a nucleic acid sequence(s) capable of effecting the expression of nucleic acid(s), or the peptide or protein product thereof. Non-limiting examples of regulatory elements include promoters, enhancers, polyadenylation signals, transcription or translation termination signals, ribosome binding sites, or other segments of DNA where regulatory proteins, such as, but not limited to, transcription factors, bind preferentially to control gene expression and thus protein expression.

Regulatory elements may be operably linked to the nucleic acids, peptides, or proteins of the described invention. When two or more elements are operably linked, there exists a functional linkage between the elements. For example, when a promoter and a protein coding sequence are operably linked, the promoter sequence initiates and mediates transcription of the protein coding sequence. The regulatory elements need not be contiguous with the nucleic acids, peptides, or proteins whose expression they control as long as they function to direct the expression thereof. Thus, for example, intervening untranslated yet transcribed sequences may be present between a promoter sequence and a nucleic acid of the described invention and the promoter sequence may still be considered operably linked to the coding sequence.

In certain embodiments, the NELL1 nucleic acid molecule is a recombinant expression cassette or is part of an expression system. The term “recombinant expression cassette” refers to a nucleic acid construct, generated recombinantly or synthetically, with a series of specified nucleic acid elements which permit transcription of a particular nucleic acid (e.g., protein coding sequence) in a host cell. The recombinant expression cassette can be incorporated into a plasmid, chromosome, mitochondrial DNA, virus, or nucleic acid fragment. Typically, the recombinant expression cassette portion of an expression vector includes, among other sequences, a nucleic acid to be transcribed, a promoter, and a transcription termination signal such as a poly-A signal.

The expression cassette or cloning vector can be generated using molecular biology techniques known in the art and utilizing restriction enzymes, ligases, recombinases, and nucleic acid amplification techniques such as polymerase chain reaction that can be coupled with reverse transcription.

In some embodiments, the NELL1 nucleic acid molecule is in a host cell that can be used for propagation of the nucleic acid molecule or for expression of the NELL1 polypeptide and subsequent isolation and/or purification. A host cell is any cell that contains a heterologous nucleic acid molecule. A heterologous polypeptide or nucleotide sequence is a polypeptide or a sequence that originates from a different species, or if from the same species, is substantially modified from its native form in composition and/or genomic locus by deliberate human intervention. The host cell typically supports the replication and/or expression of the vector. Host cells may be prokaryotic cells such as, but not limited to, Escherichia coli, or eukaryotic cells such as, but not limited to, yeast, insect, amphibian, plant (e.g., Nicotiana tabacum (tobacco), Oryza sativa (rice), Arabidopsis thaliana (cress)), or mammalian cells (e.g., Chinese hamster ovary (CHO) cells, human embryonic kidney 293-F cells). The term as used herein means any cell which may exist in culture or in vivo as part of a unicellular organism, part of a multicellular organism, or a fused or engineered cell culture. A cloning host cell is a host cell that contains a cloning vector.

A recombinant cell or vector is one that has been modified by the introduction of a heterologous nucleic acid or the cell that is derived from a cell so modified. Recombinant cells express genes that are not found in identical form within the native (non-recombinant) form of the cell or express native genes that are otherwise abnormally expressed, under-expressed or not expressed at all as a result of deliberate human intervention. The alteration of a cell or vector by naturally occurring events (e.g., spontaneous mutation, natural transformation transduction/transposition), such as those occurring without deliberate human intervention, does not result in a recombinant cell or vector.

The NELL1 nucleic acid molecule can be introduced into a host cell for propagation or production of NELL1 using any method known in the art, including transfection, transformation, or transduction, so long as the nucleic acid molecule gains access to the interior of the cell. The insertion or introduction of a nucleic acid into a cell refers to transfection or transformation or transduction and includes the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The NELL1 nucleic acid molecule can be introduced into a host cell to allow for stable transformation or transient transformation. Stable transformation is intended to mean that the nucleotide construct introduced into a cell integrates into a genome of the cell. Transient transformation is intended to mean that a polynucleotide is introduced into the cell and does not integrate into a genome of the cell.

The NELL1 polypeptide can be administered by a cell-based gene therapy. For example, autologous, allogeneic or xenogeneic donor cells are genetically modified in vitro to express and secrete the NELL1 polypeptide. The genetically modified donor cells are then subsequently implanted into the subject in need of delivery of the NELL1 polypeptide in vivo. Examples of suitable cells include, but are not limited to, skeletal satellite cells, induced pluripotent stem cells, adult mesenchymal stem cells, lung tissue precursor cells, mature, differentiated cells, and facultative progenitor cells (Kotton, D. N. and Morrisey, E. E. (2014) Nat. Med., 20(8):822-832. doi:10.1038/nm.3642).

Also contemplated herein are methods for generating or regenerating lung tissue in a 2D or 3D model of lung tissue, an organoid, or a decellularized scaffold in vitro by contacting the tissue, primary cells (e.g., progenitor cells, stem cells, induced pluripotent stem cells, adult mesenchymal stem cells, lung tissue precursor cells, mature, differentiated cells, and facultative progenitor cells), or cell lines with a NELL1 polypeptide (see, for example, Miller and Spence (2017) Physiology (Bethesda) 32(3):246-260).

### III. Methods of Treatment

The presently disclosed methods involve the treatment of virally-induced tissue damage and/or inflammation in a subject in need thereof. The terms “subject”, “individual”, and “patient” are used interchangeably to refer to a member of a species that comprises heart and lungs and is susceptible to viral infection. In certain embodiments, the subject is a mammal, including but not limited to, mouse, rat, cat, goat, sheep, horse, hamster, ferret, pig, dog, platypus, guinea pig, rabbit and a primate, such as, for example, a monkey, ape, or human. In some of these embodiments, the subject is a human, cat, dog, or a horse, such as a racehorse.

Damage to a tissue refers to harm to a tissue of the body caused by viral infection. The damage can occur directly through weakening or killing of cells due to infection of the cells by the virus, viral replication, and lysing or release of the new viral particles. The tissue damage caused by a viral infection can also be indirect due to inflammation induced by the virus. In some embodiments, a cytokine storm can be elicited by the virus wherein the body's immune system overreacts to a pathogen by releasing excessive levels of pro-inflammatory cytokines, such as IL-1, IL-6, IL-8, and TNF-α, that can lead to systemic hyperinflammation. A cytokine storm can even lead to multiple organ failure and has been the cause of a number of deaths due to COVID-19 (Mehta P et al. 2020 The Lancet 395:1033-1034). Other indirect damage induced by a viral infection can be due to ischemia or hypoxic conditions that result from damage to heart, vasculature, and/or lung tissues.

In some embodiments, the tissue that is damaged by a viral infection comprises epithelial tissue, such as those that line the respiratory tract, including the lungs.

In certain embodiments, treatment of tissue damage refers to the repair or prevention of tissue damage such that the formation of fibrotic or scar tissue is reduced or eliminated and functional tissue results. When particular tissues are insulted by various factors, such as inflammation or viral infection, the repair process can result in excessive granulation, fibrosis, or scarring that impairs function of the tissue. For example, survivors of COVID-19 that develop ARDS exhibit significant reduction of lung function (20-30% capacity) due to scarring and fibrosis (Goh K J et al. 2020 Ann Acad Med Singapore in press; Nicholls J M et al. 2020 The Lancet 361:1773; Tian S et al. 2020 J Thoracic Oncology on the world wide web at doi.org/10.1016/j.jtho.2020.02.010). NELL1 promotes wound healing or wound repair such that excessive granulation tissue formation, scarring, and fibrosis (excess deposition of extracellular matrix components) does not occur or is reduced and functional tissue results. In some embodiments, treatment of subjects that are infected by a virus with a NELL1 polypeptide or a nucleic acid molecule encoding the same can prevent the development of fibrosis, scarring, or excessive granulation due to inflammation, hypoxia, or direct infection and cellular damage due to the viral infection. For example, administration of a NELL1 polypeptide or nucleic acid molecule encoding the same to a subject infected with a respiratory virus can prevent (e.g., reduce or inhibit) fibrosis or scarring within the respiratory tract, including pulmonary fibrosis.

The alveolar sacs in the lungs are lined with alveolar epithelium comprised of alveolar type I (ATI) and type II (ATII) cells, which together form a tight barrier and protection against environmental and microbial agents that enter the lungs. 83% of ACE2-expressing cells are ATII cells, hence providing an abundant reservoir of cells for infection by viruses, such as SARS-CoV-2, that use ACE2 for entry into the cell (Zhang H et al. 2020 Intensive Care Med 46:586-590). Thus, in some embodiments, the lung tissue damage that is treated with a NELL1 polypeptide or nucleic acid molecule encoding the same comprises damage to ATII cells.

NELL1 is expressed in the regenerative lining of the lungs and plays a role in facilitating the engraftment, proliferation, and differentiation of mesenchymal stem cells to repair lung tissue and has pro-angiogenic effects via recruitment of stem cells for blood vessel formation (Pakvasa M et al. 2017 Genes & Diseases 4:127-137; Zhang X et al. 2011 Tissue Engineering: Part A 17(19-20); James A W et al. 2017 JCI Insight on the world wide web at doi.org/10.1172/jci.insight.92573; Askarinam A et al. 2013 Tissue Engineering: Part A 19(11-12)). NELL1 also inhibits the expression of various pro-inflammatory cytokines, such as IL-1β, IL-8, and TNF-α, which can lessen the negative effects of severe inflammation on lung tissues (Tisoncik J R et al. 2012 Microbiology and Molecular Biology Reviews 76(1): 16-32; Li C et al. 2020 Biomaterials 226: 119541; Mitchell D et al. 2012 Journal of the American Academy of Dermatology 66(4): Supplement 1, Page AB3; Shen J et al. 2013 Tissue Engineering: Part A 19(21-22) 2390-2401 DOI: 10.1089/ten.tea.2012.0519; Chen H et al. 2018 Brazilian J of Med and Biol Res 51(6):ee6997). NELL1 has also shown protective effects under hypoxic conditions (NellOne Therapeutics, Inc, unpublished data), which can result from damage to lung, blood vessels, and/or heart tissues. Increasing tissue survival by NELL1 under hypoxia can provide time and opportunity to initiate and sustain repair mechanisms.

Pandemics have revealed that coronaviruses attack the respiratory system and create massive organ failure called acute respiratory distress syndrome (ARDS). COVID-19, in particular, is marked by extensive damage and inflammation of the pulmonary vessels within the lungs. Oxygen exchange is severely compromised, thus lung malfunction and pneumonia ensue and escalate into life threatening respiratory failure. ARDS is linked to ˜80% of COVID-19 fatalities. Survivors exhibit significant reduction of lung function (20-30% capacity) due to scarring and fibrosis (Goh K J et al. 2020 Ann Acad Med Singapore in press; Nicholls J M et al. 2020 The Lancet 361:1773; Tian S et al. 2020 J Thoracic Oncology on the world wide web at doi.org/10.1016/j.jtho.2020.02.010). Thus, in some embodiments, the subject that is administered a NELL1 polypeptide or a nucleic acid molecule encoding the same has pneumonia, an inflammatory condition of the lung primarily affecting the alveoli. Symptoms often include cough, chest pain, fever, shortness of breath, and difficulty breathing. In some of these embodiments, the pneumonia is bilateral pneumonia, affecting both lungs. In other embodiments, the subject that is administered a NELL1 polypeptide or a nucleic acid molecule encoding the same has ARDS. ARDS can be diagnosed using clinical and ventilator criteria, which suggests an acceleration of respiratory failure (or cardiac dysfunction) and is an indication of the degree of hypoxia. In some embodiments, a NELL1 polypeptide or nucleic acid molecule encoding the same is administered at the onset of these symptoms of acute lung injury (ALI; pre-ARDS) to moderate ARDS, but is suitable for administration at later stages (with increased dosage for higher ARDS severity) until symptoms resolve. For example, in lung injury a standard parameter is the ratio of partial arterial pressure of oxygen (PaO₂) to the fractional concentration of oxygen in inspired air (FiO₂). The severity of hypoxemia distinguishes the patient classification as follows (Cascella M et al. 2020 on the world wide web at ncbi.nlm.nih.gov/books/NBK554776/; Proudfoot A G et al. 2011 Disease Models and Mechanisms 4:145-153 doi:10.1242/dmm.006213; Ragaller M and Richter T 2010 J Emerg Trauma Shock 3(1):43-51 doi:10.4103/0974-2700.58663):

Acute Lung Injury (ALI) is an acute lung disease characterized by bilateral infiltrate in a radiograph consistent with edema and no evidence of left atrial hyptertension; pulmonary wedge pressure of 18 mmHg or less; the PaO₂/FiO₂ is 300 mmHg or 40 kPa or less, regardless of PEEP (positive end-expiratory pressure) value. ARDS is considered the most severe form of ALI and defined by PaO₂/FiO₂ of 200 mmHg or less, with subclasses as follows:

Moderate ARDS: 100 mmHg<PaO₂/FiO₂≤200 mmHg

Severe ARDS: PaO₂/FiO₂≤100 mm Hg

In addition to the ventilation criteria, clinical imaging data from chest radiographs, CT scans, and/or lung ultrasounds are used to detect the hallmarks of lung injury, which are bilateral opacities indicating lung infiltrates>50% that cannot be entirely attributed to effusions, lobar or lung collapse.

Thus, in some embodiments, the subject that is in need of a NELL1 polypeptide or nucleic acid encoding the same exhibits a PaO₂/FiO₂ ratio of less than 300 mmHg or less than 40 kPa, which is indicative of ALI. In other embodiments, the subject has a PaO₂/FiO₂ ratio of greater than 200 mmHg and less than 300 mmHg, including but not limited to about 205 mmHg, about 210 mmHg, about 220 mmHg, about 230 mmHg, about 240 mmHg, about 250 mmHg, about 260 mmHg, about 270 mmHg, about 280 mmHg, about 290 mmHg, and about 300 mmHg. These subjects are non-ventilated or are being treated with non-invasive ventilation. In still other embodiments, the subject that is in need of a NELL1 polypeptide or nucleic acid encoding the same exhibits a PaO₂/FiO₂ ratio of less than or equal to 200 mmHg, but greater than 100 mmHg, including but not limited to about 105 mmHg, about 110 mmHg, about 120 mmHg, about 130 mmHg, about 140 mmHg, about 150 mmHg, about 160 mmHg, about 170 mmHg, about 180 mmHg, about 190 mmHg, and about 200 mmHg, which is indicative of moderate ARDS. In yet other embodiments, the subject that is in need of a NELL1 polypeptide or nucleic acid encoding the same exhibits a PaO₂/FiO₂ ratio of less than or equal to 100 mmHg, which is indicative of severe ARDS.

In some embodiments, administration of a NELL1 polypeptide or nucleic acid can reduce, therapeutically or prophylactically, the number and size of lung infiltrates by at least 10%, at least 20%, at least 30%, at least 40%, at least 50%, at least 60%, at least 70%, at least 80%, at least 90%, or more when compared to the same subject prior to administration of the NELL1 polypeptide or nucleic acid or compared to a suitable control (e.g., the subject prior to NELL1 treatment, a subject having similar symptoms that has not been treated with NELL1, or an average number).

In certain embodiments, administration of a NELL1 polypeptide or nucleic acid can increase the PaO₂/FiO₂ ratio, therapeutically or prophylactically, by at least 10%, at least 20%, at least 30%, at least 40%, at least 50%, at least 60%, at least 70%, at least 80%, at least 90%, or more when compared to the same subject prior to administration of the NELL1 polypeptide or nucleic acid or compared to a suitable control (e.g., the subject prior to NELL1 treatment, a subject having similar symptoms that has not been treated with NELL1, or an average number). In some of these embodiments, administration of a NELL1 polypeptide or nucleic acid to a subject having ALI can increase the PaO₂/FiO₂ ratio above 300 mmHg or above 40 kPa.

In optimal patients (ALI and mild ARDS), successful treatment with a NELL1 polypeptide or nucleic acid molecule encoding the same promotes healing so symptoms resolve before ventilation is required. In certain embodiments, however, the subject is on supplementary oxygen or on artificial ventilation wherein mechanical means are used to assist or replace spontaneous breathing, such as a ventilator machine or manual assistance using, for example, a bag valve mask device. Mechanical ventilation can be positive-pressure or negative-pressure ventilation.

Mechanical ventilation is required in up to 80% of COVID-19 patients and can cause further damage called ventilator-associated lung injury (VALI). In situations with patients already on ventilation, successful intervention with NELL1 accelerates healing so ventilation time is significantly reduced, thereby lowering mortality rate or long-term neuromuscular and psychological effects. Typically, most ALI/ARDS patients either pass away or are weaned off ventilation within 1-2 weeks, and others need 30 days or more (Proudfoot A G et al. 2011 Disease Models and Mechanisms 4:145-153 doi:10.1242/dmm.006213). In some embodiments, administration of a NELL1 polypeptide or nucleic acid to a subject in need thereof can reduce the amount of time needed on ventilation as compared to an appropriate control (e.g., a subject having similar symptoms that has not been treated with NELL1, or an average number). In some of these embodiments, administration of a NELL1 polypeptide or nucleic acid to a subject can reduce the amount of time needed on ventilation by at least 10%, at least 20%, at least 30%, at least 40%, at least 50%, at least 60%, at least 70%, at least 80%, at least 90%, or more when compared to an appropriate control.

In addition to lung injuries, viral infection can also cause heart and vascular tissue damage. Damage to heart or vascular tissues by viruses can be the result of direct invasion of heart or endothelial tissues or indirectly via the powerful cytokine storm that subjects the tissues to severely elevated cytokine levels (e.g., IL1, IL6, TNF-alpha, NF-κb) and attack by pro-inflammatory cells from an exaggerated immune response (Tisoncik J R et al. 2012 Microbiology and Molecular Biology Reviews 76(1): 16-32; Wu C et al. 2020 medRxiv preprint on the world wide web at doi.org/10.1101/2020.02.26.20028589; Clerkin K J et al. 2020 Circulation doi: 10.1161/CIRCULATIONAHA.120.046941; Zheng Y Y et al. 2020 Nature Reviews Cardiology on the world wide web at doi.org/10.1038/41569-020-0360-5). Multiple direct and indirect cardiovascular complications can manifest clinically as acute myocardial injury, myocarditis, arrhythmias, and venous thromboembolism, thereby presenting tremendous challenges to both patients and medical personnel during diagnosis and treatment of a viral infection (Driggin E et al. 2020 Journal of the American College of Cardiology on the world wide web at doi org/10.1016/j.jacc.2020.03.031; Bonow R O et al. 2020 JAMA Cardiology doi:10.1001/jamacardio.2020.1105). Similar to the assault on the epithelial linings of the respiratory system, the direct effects of SARS-CoV-2, at least partially, are exerted by the binding of the virus to the angiotensin converting enzyme 2 (ACE2) receptor in the heart muscle cells (Zou X et al. 2020 Front. Med. On the world wide web at journal.hep.com.cn/fmd/EN/10.1007/s11684-020-0754-0; Xu H et al. 2020 International Journal of Oral Science on the world wide web at doi.org/10.1038/s41368-020-0074-x). Viral particles that gain entry via receptor mediated endocytosis shed their protein coating and hijack the cell machinery for replicating the viral RNA and expressing the protein components of the protein coat. The viral particles assemble and are released from the cells via exocytosis. (Cascella M et al. 2020 on the world wide web at ncbi.nlm.nih.gov/books/NBK554776/).

In COVID-19 patients, in addition to any method known in the art to detect myocardial injury (e.g., electrocardiogram, measurements of creatine kinase MB), evidence of myocardial injury can be detectable by elevation of high-sensitivity cardiac troponin 1 (hs-cTn1) and Troponin T (TnT) levels. Two patient cohorts from Wuhan, China indicated about 20-28% of patients have evidence of myocardial injury and exhibited 50-60% of hospital mortality rates. Moreover, myocardial injury was linked to severe systemic inflammation, greater leukocyte counts, higher levels of C reactive protein, procalcitonin, creatine kinase, myoglobin, and NT-proBNP (Bonow R O et al. 2020 JAMA Cardiology doi:10.1001/jamacardio.2020.1105; Zheng Y Y et al. 2020 Nature Reviews Cardiology on the world wide web at doi.org/10.1038/41569-020-0360-5). This severe heart tissue injury is also associated with the early mortality cases of COVID-19 (Wu C et al. 2020 medRxiv preprint on the world wide web at doi.org/10.1101/2020.02.26.20028589). Thus, in some embodiments, the virally-infected subject in need of treatment with a NELL1 polypeptide or a nucleic acid molecule encoding the same has heart damage, which in some embodiments, can be diagnosed by high levels of hs-cTn1 and/or TnT when compared to a healthy patient (e.g., one not experiencing a viral infection and/or one not experiencing cardiovascular symptoms). In some of these embodiments, the subject has greater than 0.4 ng/ml of hs-cTN1 as measured in the blood, including but not limited to about 0.5 ng/ml, about 0.6 ng/ml, about 0.7 ng/ml, about 0.8 ng/ml, about 0.9 ng/ml, about 1.0 ng/ml, about 1.5 ng/ml, about 2 ng/ml, or higher. In some embodiments, the subject has greater than 14 ng/1 of TnT as measured in the blood, including but not limited to about 15 ng/l, about 16 ng/l, about 17 ng/l, about 18 ng/l, about 19 ng/l, about 20 ng/l, about 25 ng/l, about 30 ng/l or higher. In some embodiments, administration of a NELL1 polypeptide or nucleic acid can reduce myocardial injury or vasculature injury in virally infected subjects when given prophylactically or therapeutically. In some of these embodiments, evidence of myocardial injury is reduced by at least 10%, at least 20%, at least 30%, at least 40%, at least 50%, at least 60%, at least 70%, at least 80%, at least 90%, or more in subjects administered NELL1 polypeptide or nucleic acid when compared to an appropriate control (e.g., the subject prior to NELL1 treatment, a subject having similar symptoms that has not been treated with NELL1, or an average number). In certain embodiments, administration of a NELL1 polypeptide or nucleic acid can reduce levels of hs-cTn1 and/or TnT by at least 10%, at least 20%, at least 30%, at least 40%, at least 50%, at least 60%, at least 70%, at least 80%, at least 90%, or more when compared to an appropriate control (e.g., the subject prior to NELL1 treatment, a subject having similar symptoms that has not been treated with NELL1, or an average number).

SARS-CoV-2 infection has been shown to cause damage to blood vessels and endothelial cells, leading to vasculature leakage, widespread thrombosis and microangiopathy. Vascular effects of SARS-CoV-2 and other coronaviruses may be due to direct binding of the virus to endothelial cells (that express the ACE2 receptor) and killing thereof, or indirect damage as the result of hyperinflammation. Administration of a NELL1 polypeptide or nucleic acid can treat (therapeutically or prophylactically) viral damage to blood vessels, at least in part, due to its pro-angiogenic and anti-inflammatory effects. In some embodiments, administration of an effective amount of a NELL1 polypeptide or nucleic acid can reduce damage to the vasculature or regenerate blood vessels via angiogenesis, leading to a reduction in the number or size of blood clots and strokes in subjects of at least 10%, at least 20%, at least 30%, at least 40%, at least 50%, at least 60%, at least 70%, at least 80%, at least 90%, or more when compared to an appropriate control (e.g., the subject prior to NELL1 treatment, a subject having similar symptoms that has not been treated with NELL1, or an average number).

It is noteworthy that although an abnormal inflammatory response (e.g. chronic and does not return to normal levels) is often observed in cardiovascular injury and disease, the induction of a cytokine storm during viral infection that damages heart tissue is a distinct inflammatory process or environment for tissues. It is sudden and catastrophic (extreme levels of cytokines and soft tissue injury markers), involves a subset of pro-inflammatory cytokines that interact with each other, and other molecules and cells of the immune system in novel processes and dynamics that are still mostly unknown (Tisoncik J R et al. 2012 Microbiology and Molecular Biology Reviews 76(1): 16-32).

Subjects that could benefit from treatment with a NELL1 polypeptide or nucleic acid molecule encoding the same include those that have a viral infection that has triggered a cytokine storm. In some embodiments, the cytokine storm involves elevated levels in the subject (when compared to a control subject not infected by a virus) of at least one of the following cytokines: interleukin-6 (IL-6), IL-1, IL-1ra, IL-2R, IL-2ra, IL-10, IL-18, hepatocyte growth factor (HGF), interferon-gamma (IFN-γ), tumor necrosis factor-alpha (TNF-α), CCL-2/MCP-1, CXCL-10/interferon gamma induced protein 10 (IP-10), monocyte chemotactic protein-3 (MCP-3), macrophage inflammatory protein 1 alpha (MIG-1a), macrophage colony stimulating factor (M-CSF), granulocyte colony-stimulating factor (G-CSF), and cutaneous T-cell-attracting chemokine (CTACK).

Specific criterion used for identifying patients for NELL1 therapy is the level of key cytokines in the cytokine storm that injures the lung tissue. An example is the level of IL-6 which was found to be predictive of respiratory failure and the need for mechanical ventilation in hospitalized symptomatic COVID-19 patients (Herold T et al. 2020 medRxiv on the worldwide web at doi.org/10.1101/2020.04.01.20047381). The maximal IL-6 level (cutoff at 80 pg/ml) per patient during the disease progression indicated respiratory failure with high accuracy and when patients reached IL-6 levels of ≥80 pg/ml, the patients were 22 times more likely to experience respiratory failure. Thus, in some embodiments, the subject in need of treatment with a NELL1 polypeptide or nucleic acid encoding the same is one having at least about 80 pg/ml of IL-6, as measured in the subject's blood, plasma, or serum, including but not limited to about 80 pg/ml, about 85 pg/ml, about 90 pg/ml, about 95 pg/ml, about 100 pg/ml, or higher. In other embodiments, the subject that is administered a NELL1 polypeptide or nucleic acid molecule is one that has at least 5 ng/ml of IP-10, as measured in the subject's blood, plasma, or serum, including but not limited to about 5 ng/ml, about 6 ng/ml, about 7 ng/ml, about 8 ng/ml, about 9 ng/ml, about 10 ng/ml, or higher.

Other tissues that can be damaged by viruses that can benefit from the administration to the subject of a NELL1 polypeptide or nucleic acid encoding the same include kidney, esophagus, oral mucosa, intestine, and skeletal muscle.

As demonstrated herein (see Example 1), SARS-CoV-2 infection of a transgenic mouse expressing human ACE2 caused an immediate decrease in body weight within a day post infection that continued to decrease until death or the animal was euthanized. Administration of NELL1 on days 0 and 3 post infection protected the SARS-CoV-2-infected hACE2 transgenic mice from the virus-induced weight loss. Patients with COVID-19 have also been shown to exhibit weight loss (see e.g., Filippo et al. (2020) Clinical Nutrition, doi.org/10.1016/j.clnu.2020.10.043; and Morley et al. (2020) Journal of Cachexia, Sarcopenia and Muscle 11:863-865). The weight loss associated with SARS-CoV-2 infection could, at least partially, be due to loss of skeletal muscle or skeletal muscle atrophy, possibly associated with the hyperinflammation and cytokine storm induced by the virus (see Morley et al. (2020)).

Muscle atrophy may refer to a disease or condition characterized by the decrease in the mass of a muscle, fiber size, cross-sectional area, or other muscle characteristic in a subject and/or a progressive weakening and degeneration of muscle tissue. A decrease in the mass of the muscle is usually accompanied with a weakening of the muscles (i.e. decreasing muscle function). In some embodiments, muscle atrophy may refer to a decrease in a muscle characteristic (e.g., mass) of at least about 10%, at least about 15%, at least about 20%, at least about 25%, at least about 30%, at least about 35%, at least about 40%, at least about 45%, at least about 50%, or more relative to the same muscle tissue in a healthy/normal subject (i.e. a control subject) or population of healthy/normal individuals (e.g., relative to average, medium, or minimum threshold values) or relative to a recorded or estimated baseline value in the subject. Symptoms of muscle atrophy can include impaired muscle coordination, smaller appearance of muscles, muscle fatigue, muscle weakness, and impaired balance. These symptoms, such as muscle strength, may be measured by an appropriate test known in the art.

The protection from virus-induced weight loss demonstrated in the COVID-19 animal model with NELL1 could, in part, be attributed to the direct regenerative effects of NELL1 on skeletal muscle or indirect effects of NELL1 dampening the inflammatory response. Without being bound by any theory or mechanism of action, it is believed that NELL1's benefits to skeletal muscle occur by addressing both muscle breakdown (e.g., muscle protein degradation) and formation pathways (e.g., increase in muscle mass, increased fusion of satellite cells, increase in muscle protein synthesis). Specifically, it is believed to reduce potent pro-inflammatory molecules that trigger protein degradation and subsequent muscle loss. NELL1 is also believed to promote muscle formation and maintenance via the production of certain extracellular matrix proteins that mediate regeneration and impart muscle function and strength and in some cases, through the promotion of muscle precursor cells (e.g., skeletal satellite cell, osteoblast precursor, perivascular stem cell) to maturity.

In some embodiments, administration of a NELL1 polypeptide or nucleic acid results in a detectable and sufficient increase in one or more of quantifiable muscle characteristics, such as muscle mass, fiber size, cross-sectional area, strength, power, or other functional measurement. In some embodiments, total body weight may be used to quantify the results of treatment. In some embodiments, the muscle mass or muscle characteristics of one or more particular muscles may be used to quantify the results of treatment (e.g., tibialis anterior muscle mass, gastrocnemius muscle mass, quadriceps muscle mass, biceps trachii muscle mass, triceps trachii muscle mass, deltoid muscle mass, etc.). In some of these embodiments, administration of a NELL1 polypeptide or nucleic acid results in at least about 10%, 15%, 20%, 25%, 30%, 40%, 50%, 60% or more improvement relative to the same measure (e.g., increase in muscle mass, fiber size, cross-sectional area, strength, power, or body weight) in the subject prior to the treatment, relative to a predicted prognosis without treatment, or relative to a control subject who did not receive treatment.

Methods for treating weight loss or muscle atrophy due to a viral infection in a subject in need thereof are provided. The muscle atrophy can be cardiac muscle atrophy and/or skeletal muscle atrophy. The method comprises administering to the subject an effective amount of a NELL1 polypeptide, or a nucleic acid molecule encoding the same.

Muscle dysfunction is common in patients with ARDS which can be caused by respiratory viruses, such as influenza A (Radigan et al. (2019) J Immunol 202:484-493) and SARS-CoV-2. Thus, in some of these embodiments, the viral infection is an infection by a respiratory virus. In certain embodiments, the viral infection is an infection by a coronavirus. In some of these embodiments, the coronavirus is SARS-CoV-2.

Treating a subject refers to the administering of the NELL1 polypeptide or a nucleic acid molecule encoding the same to a subject for a therapeutic or prophylactic purpose. Administration may include any method of delivery of the NELL1 polypeptide or nucleic acid molecule encoding the same into the subject's system or to a particular region in or on the subject (i.e., systemic or local administration).

Treatment of virally-induced tissue damage with a NELL1 polypeptide or nucleic acid molecule encoding the same can result in a partial or complete recovery of tissue and function thereof or a partial or complete prevention of symptoms associated with tissue damage. Thus, treatment of virally-induced tissue damage with a NELL1 polypeptide or nucleic acid molecule encoding the same can result in at least about 10%, at least about 20%, at least about 25%, at least about 30%, at least about 40%, at least about 50%, at least about 60%, at least about 70%, at least about 75%, at least about 80%, at least about 85%, at least about 90%, at least about 95%, or more recovery of tissue (e.g., area) or function thereof in a subject experiencing tissue damage or infected with a virus. The onset of tissue damage can also be delayed or the associated symptoms lessened through prophylactic treatment with a NELL1 polypeptide or nucleic acid molecule encoding the same.

Treatment may include prophylactic treatment of subjects not presently exhibiting symptoms of tissue damage. In other embodiments, subjects that exhibit symptoms of viral infection or have been diagnosed with a viral infection are treated with a NELL1 polypeptide or nucleic molecule encoding the same in order to prevent tissue damage or any additional tissue damage.

In some embodiments, subjects who are at risk of developing virally-induced tissue damage (e.g., have an increased likelihood relative to a general population of subjects) or who show mild or moderate signs or symptoms of a viral infection and tissue damage (i.e., and are at risk for progressing to a more severe state) may be treated. Subjects suitable for prophylactic treatment includes those that are exhibiting symptoms or have been diagnosed with a viral infection and have a previously diagnosed cardiovascular or pulmonary disorder, including but not limited to chronic obstructive pulmonary disease, a history of smoking, diabetes, high blood pressure, high cholesterol, coronary heart disease, congenital heart disease, myocardial infarction, pericardial disease, stroke, vascular disease, asthma, pneumonia, pneumothorax, pneumonitis, interstitial lung disease, and lung cancer.

Subjects in need of treatment for virally-induced tissue damage include those that have been diagnosed with a viral infection either through tests that detect the presence of a particular virus within the subject or the manifestation of symptoms associated with viral infection. Viral infection can be tested using any method known in the art, including blood tests, such as full blood count (a viral infection may raise or reduce the white cell count or atypical lymphocytes may be reported), C-reactive protein measurement (a marker of inflammation), assays (e.g., enzyme-linked immunosorbent assay) that measure antibodies specific to a particular virus, nucleic acid detection assays such as polymerase chain reaction that detect viral specific nucleic acid sequences (RNA or DNA), or viral cell culture. In the case of suspected SARS-CoV-2 infection, the virus can be identified by detection of viral-specific RNA. The presence of the virus can be measured in biological samples from a subject. In those embodiments wherein the suspected virus is a respiratory virus, the presence of the virus can be measured in a mucosal sample, such as a nasopharyngeal swab. Thus, the presently disclosed methods can include a step of testing for the presence of a viral infection using any method known in the art.

Symptoms of a viral infection can also identify those patients that would benefit from treatment with a NELL1 polypeptide or nucleic acid encoding the same. Symptoms of a viral infection include but are not limited to fever, body aches, fatigue, chills, diarrhea, vomiting, cough, headache, sore throat, and nasal congestion. Symptoms of SARS-CoV-2 infection specifically include shortness of breath, trouble breathing, fever, dry cough, chills, body aches, sudden confusion, diarrhea, conjunctivitis, loss of smell and/or taste, fatigue, headache, sore throat, and nasal congestion.

A NELL1 polypeptide or nucleic acid molecule encoding the same can be administered to a subject after the subject tests positive for viral infection or is presumed positive based on contact with an infected individual or based on symptoms. In some embodiments, the subject is administered a NELL1 polypeptide or nucleic acid encoding the same within about 1 hour, about 2 hours, about 3 hours, about 4 hours, about 5 hours, about 6 hours, about 7 hours, about 8 hours, about 9 hours, about 10 hours, about 12 hours, about 20 hours, about 1 day, about 2 days, about 3 days, about 4 days, about 5 days, about 6 days, about 1 week, about 2 weeks, or more after testing positive for a viral infection (e.g., COVID-19).

A NELL1 polypeptide or nucleic acid molecule encoding the same can be administered to a subject that has experienced virally-induced tissue damage or exhibits symptoms thereof in order to regenerate the damaged tissues. NELL1 has regenerative properties in many tissues, including epithelial tissues, such as those in lungs. Thus, provided herein is a method of regenerating lung tissue in a subject with damaged lung tissue by administering to a subject in need thereof an effective amount of a NELL1 polypeptide or a nucleic acid molecule encoding the same. In some embodiments, the subject that is administered a NELL1 polypeptide or nucleic acid molecule encoding the same is one that is at risk of impaired healing, such as one with impaired angiogenesis, diabetes, or a history of smoking.

Also provided herein are methods of treating lung inflammation (i.e., pneumonitis) in a subject by administering to a subject in need thereof an effective amount of a NELL1 polypeptide, or a nucleic acid molecule encoding the same. Pneumonitis can be caused by certain drugs (e.g., chemotherapeutics), molds, bacteria, viruses, exposure to bird feathers or excrement, radiation therapies, smoking, vaping. In some of these embodiments, the lung inflammation is virally-induced and is thus viral pneumonia. Lung inflammation can be diagnosed using any method known in the art, including but not limited to, chest X-ray or CT, bronchoalveolar lavage with lymphocytosis particularly with a low CD4:CD8 ratio, and lung biopsy consistent with pneumonitis histopathology.

In some embodiments, a subject is administered a heterologous NELL1 polypeptide or nucleic acid molecule, meaning the NELL1 polypeptide or nucleic acid molecule is derived from a species different from the subject or has been substantially modified from its native form in composition and/or genomic locus by deliberate human intervention. For example, the NELL1 polypeptide comprises a mutation not present in the NELL1 polypeptide of the subject or comprises a non-naturally occurring amino acid residue or was produced in a different species or an in vitro translation system and thus comprises altered glycosylation patterns from the native protein. In other embodiments, the nucleic acid molecule encoding a NELL1 polypeptide comprises regulatory sequences or vector sequences not found in the subject or the NELL1 genomic locus of the subject. In those instances wherein a nucleic acid molecule encoding a NELL1 polypeptide is administered to a subject or formulated for administration, the nucleic acid molecule can be in the form of an expression vector or viral vector (e.g., retroviral vector, adenoviral vector, adeno-associated viral vector) or can be delivered encapsulated within a liposome, nanoparticle (e.g., lipid nanoparticle), or exosome. A NELL1 polypeptide may also be delivered within a nanoparticle (e.g., lipid nanoparticle), liposome, or exosome.

The NELL1 polypeptide or nucleic acid molecule encoding the same can be administered to subjects in need thereof in the form of a composition further comprising a carrier. The term “carrier” as used herein describes a material that does not cause significant irritation to an organism and does not abrogate the biological activity and properties of the NELL1 polypeptide or nucleic acid molecule encoding the same. Carriers must be of sufficiently high purity and of sufficiently low toxicity to render them suitable for administration to a subject being treated. The carrier can be inert, or it can possess pharmaceutical benefits.

In some embodiments, the NELL1 polypeptide or nucleic acid encoding the same is administered to a subject in the form of a pharmaceutical composition. A pharmaceutical composition is a composition that is employed to prevent, reduce in intensity, cure or otherwise treat a target condition or disease that comprises an active ingredient (i.e., NELL1 polypeptide or nucleic acid molecule encoding the same) and a pharmaceutically acceptable carrier. A pharmaceutically acceptable carrier refers to one or more compatible solid or liquid filler, diluents or encapsulating substances which are suitable for administration to a human or other vertebrate animal.

Pharmaceutical compositions used in the presently disclosed methods can be formulated with suitable carriers, excipients, and other agents that provide suitable transfer, delivery, tolerance, and the like. A multitude of appropriate formulations are known to those skilled in the art. Suitable formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Pharmaceutical compositions for oral or parenteral use may be prepared into dosage forms in a unit dose suited to fit a dose of the active ingredients. Such dosage forms in a unit dose include, for example, tablets, pills, capsules, injections (ampoules), suppositories, etc.

The NELL1 polypeptide or nucleic acid molecule encoding the same may be mixed with other active materials that do not impair the desired action, or with materials that supplement the desired action. In certain embodiments, the NELL1 polypeptide or nucleic acid molecule encoding the same may be mixed or attached to molecules that target the active ingredient to particular tissues or increase its stability and persistence in blood, tissues, or other bodily fluids. Solutions or suspensions used for parenteral, intradermal, subcutaneous, intrathecal, or topical application may include, but are not limited to, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. Administered intravenously, particular carriers are physiological saline or phosphate buffered saline (PBS). In some embodiments, the NELL1 polypeptide or nucleic acid molecule is PEGylated, for example, as described in Tanjaya et al. (2018) Am J Pathol 188:715-727; Zhang et al. (2014) Biomaterials 35:6614-6621; and Kwak et al. (2015) Biomaterials 57:73-83, the formulation described in each is herein incorporated by reference.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. In some embodiments, the NELL1 polypeptide or nucleic acid molecule encoding the same is administered as an injectable material in buffered liquid solution, and in some of these embodiments, with protein stabilizers. The formulation may be frozen and later thawed for injection or kept stabilized under refrigeration or room temperature prior to use. The NELL1 polypeptide or nucleic acid molecule encoding the same can be formulated as a lyophilized powder to be inhaled and/or reconstituted with liquid (e.g., buffered saline solution).

The NELL1 polypeptide or nucleic acid molecule encoding the same can also be administered orally as pills, tablets, or capsules, and in some of these embodiments, the pills, tablets, or capsules can have different release properties.

In some embodiments, the NELL1 polypeptide or nucleic acid molecule is administered via any method that delivers the polypeptide or nucleic acid molecule to the lungs, such as nasal or oral inhalation. The NELL1 polypeptide or nucleic acid molecule must be atomized into droplets for administration via inhalation. Formulations intended for oral inhalation require atomization into smaller droplets than those intended for administration by the nasal route. In some embodiments, the NELL1 polypeptide or nucleic acid molecule is administered using a nebulizer or inhaler device.

Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions also may contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

The NELL1 polypeptide or nucleic acid molecule encoding the same can also be directly linked with molecules that allow slow release and/or increase protein stability or persistence (i.e., half-life) in the circulatory system.

Injectable depot forms can be made by forming microencapsulated matrices of the drug in biodegradable polymers such as polylactide-polyglycolide. Depending upon the ratio of drug to polymer and the nature of the particular polymer employed, the rate of drug release may be controlled. Such long acting formulations may be formulated with suitable polymeric or hydrophobic materials (for example as an emulsion in an acceptable oil) or ion exchange resins, or as sparingly soluble derivatives, for example, as a sparingly soluble salt. Examples of other biodegradable polymers include poly(orthoesters) and poly(anhydrides). Depot injectable formulations can also be prepared by entrapping the drug in liposomes or microemulsions which are compatible with body tissues.

The formulations may be sterilized, for example, by filtration through a bacterial-retaining filter or by incorporating sterilizing agents in the form of sterile solid compositions that may be dissolved or dispersed in sterile water or other sterile injectable medium just prior to use. Injectable preparations, for example, sterile injectable aqueous or oleaginous suspensions, may be formulated according to the known art using suitable dispersing or wetting agents and suspending agents. The sterile injectable preparation also may be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils conventionally are employed or as a solvent or suspending medium. For this purpose, any bland fixed oil may be employed including synthetic mono- or diglycerides. In addition, fatty acids such as oleic acid are used in the preparation of injectables.

Formulations for parenteral (including but not limited to, subcutaneous, intradermal, intramuscular, intravenous, intraperitoneal, intrathecal intra-arterial, and intraarticular) administration include aqueous and non-aqueous sterile injection solutions that may contain anti-oxidants, buffers, bacteriostats and solutes, which render the formulation isotonic with the blood of the intended recipient; and aqueous and non-aqueous sterile suspensions, which may include suspending agents and thickening agents. The formulations may be presented in unit-dose or multi-dose containers, for example sealed ampules and vials, and may be stored in a freeze-dried (lyophilized) condition requiring the addition of the sterile liquid carrier, for example, saline, water-for-injection, a semi-liquid foam, or gel, immediately prior to use. Extemporaneous injection solutions and suspensions may be prepared from sterile powders, granules and tablets of the kind previously described. Alternatively, a NELL1 polypeptide or nucleic acid encoding the same is dissolved in a buffered liquid solution that is frozen in a unit-dose or multi-dose container and later thawed for injection or kept/stabilized under refrigeration until use.

The therapeutic agent(s) may be contained in controlled release systems. In order to prolong the effect of a drug, it often is desirable to slow the absorption of the drug from subcutaneous, intrathecal, or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. In some embodiments, the use of a long-term sustained release implant may be particularly suitable for treatment of chronic conditions. Long-term sustained release implants are well-known to those of ordinary skill in the art.

The NELL1 polypeptide or nucleic acid molecule encoding the same can be administered to a subject by dispensing, supplying, applying, or giving the NELL1 polypeptide or nucleic acid molecule encoding the same to the subject. Generally, NELL1 polypeptides, nucleic acid molecules encoding the same, or compositions comprising the NELL1 polypeptide or nucleic acid may be administered systemically either orally, buccally, parenterally, topically, by inhalation or insufflation (i.e., through the mouth or through the nose), or rectally in dosage unit formulations, optionally containing the conventional nontoxic pharmaceutically acceptable carriers, adjuvants, and vehicles as desired, or may be locally administered by means such as, but not limited to, injection, implantation, grafting, or topical application. Additional administration may be performed, for example, intravenously, transmucosally, transdermally, intramuscularly, subcutaneously, intraperitoneally, intrathecally, intralymphatically, intra-arterially, intralesionally, or epidurally.

Any suitable route of administration may be used to deliver the NELL1 polypeptide or nucleic acid molecule encoding the same for the purposes of treating virally-induced tissue damage and/or inflammation. In some of these embodiments, the NELL1 polypeptide, NELL1 nucleic acid molecule, or a composition comprising the NELL1 polypeptide or NELL1 nucleic acid molecule are administered parenterally. The term “parenteral” as used herein refers to introduction into the body by way of an injection (i.e., administration by injection), including, for example, subcutaneously (i.e., an injection beneath the skin beneath the dermis into the subcutaneous tissue or “superficial fascia”), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques. A parenterally administered composition is delivered using a needle, e.g., a surgical needle. Injectable preparations, such as sterile injectable aqueous or oleaginous suspensions, may be formulated according to the known art using suitable dispersing or wetting agents and suspending agents. According to some such embodiments, the NELL1 polypeptide or nucleic acid molecule encoding the same is administered by injection.

Administering can be performed, for example, once, a plurality of times, and/or over one or more extended periods. Generally, an effective dose of the NELL1 polypeptide or nucleic acid encoding the same is administered to a subject one or more times. In certain preferred embodiments, the course of treatment will comprise multiple doses of the NELL1 polypeptide or nucleic acid encoding the same over a period of days, weeks or months. More specifically, the NELL1 polypeptide or nucleic acid encoding the same may be administered once every day, every two days, every three days, every four days, every five days, every six days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

An effective amount of a pharmaceutical composition of the invention is any amount that is effective to achieve its purpose (e.g., prevention of or recovery from, including partial recovery, or prevention or slowing of tissue damage and/or inflammation). The effective amount, usually expressed in mg/kg can be determined by routine methods during pre-clinical and clinical trials by those of skill in the art. The effective amount refers to a dose of the NELL1 polypeptide or nucleic acid molecule encoding the same that results in

For instance, in some embodiments, an effective amount will include an amount providing at least about 10%, 15%, 20%, 25%, 30%, 40%, 50%, 60% or more improvement relative to the same measure in the subject prior to the treatment, relative to a predicted prognosis without treatment, or relative to a control subject who did not receive treatment. An effective amount with respect to the NELL1 polypeptide or nucleic acid encoding the same can mean the amount of polypeptide (or nucleic acid) alone, or in combination with other therapies, that provides a therapeutic or prophylactic benefit in the treatment or management of virally-induced tissue damage and/or inflammation, which can include a decrease in severity of symptoms associated with virally-induced tissue damage and/or inflammation, an increase in frequency and duration of symptom-free periods, or a prevention of symptoms. In some embodiments, an effective amount of NELL1 polypeptide may comprise a dose administered between about 0.0001-100 mg/kg of the subject body weight (e.g., 0.0001 mg/kg, 0.0005 mg/kg, 0.001 mg/kg, 0.005 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.10 mg/kg, 0.20 mg/kg, 0.30 mg/kg, 0.40 mg/kg, 0.5 mg/kg, 0.6 mg/kg, 0.7 mg/kg, 0.8 mg/kg, 0.9 mg/kg, 1.0 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, etc.).

Treatments disclosed herein may be administered to a subject in a single dose or as multiple doses over a period of time. For instance, the treatments may be administered over a defined time course according to a treatment regimen. Doses of treatment may be administered sequentially, meaning each of the doses is administered to the subject at a different point in time, e.g., separated by a predetermined interval of hours, days, weeks, or months. For example, in some embodiments, a subsequent dose may be administered 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after the immediately preceding dose. In some embodiments, two or more doses (e.g., all of the doses) may comprise the same amount of active ingredient (i.e. NELL1 polypeptide or nucleic acid molecule encoding the same). In some embodiments, the amount of each dose may be modulated (increased or decreased) over time according to a predetermined regimen and/or according to the subject's response to treatment. For instance, the dosage may be increased in subjects who do not display sufficiently improved measurements or outcomes or, alternatively, the dosage may be decreased in subjects who display adverse side effects.

The NELL1 polypeptide or nucleic acid molecule encoding the same can be administered prior to, along with, or subsequent to another treatment for virally-induced tissue damage and/or inflammation, including one or more additional therapeutic agents (i.e. active ingredients). Combination therapy generally refers to co-administration of two or more biologically active agents (e.g., drugs) used in conjunction with each other. Combination therapy may comprise a single formulation or multiple formulations. In some embodiments, combination therapies may include 2, 3, 4, 5, or more individual therapies. Co-administration may be carried out as concurrent administration or serial administration. Co-administration may be carried out via the same route of administration or different routes of administration. In some embodiments, combination therapeutic agents may be administered via the same carrier (e.g., a pharmaceutically acceptable carrier). In some embodiments, combination therapeutic agents may be administered via separate carriers or vehicles, whether administered substantially simultaneously or sequentially. Combination therapy may include two or more therapies in which the effects overlap in the subject for purposes of achieving supplemental or additive synergistic clinical effects. In some implementations, the dosage, the effective amount, and/or the administration regimen of an individual therapeutic agent (e.g., the NELL1 polypeptide) may be adjusted relative to the dosage, the effective amount, and/or the administration regimen of the therapeutic agent when delivered alone (i.e. not as part of a combination therapy). For instance, the dosage, the effective amount, and/or the frequency of administration may be reduced. In other embodiments, the dosage, the effective amount, and/or the administration regimen may remain substantially the same.

NELL1 can be combined with cells that are important in the formation of specific tissues. Cells may be naturally extracted from the subject, an allograft, or a xenograft or may be synthetically engineered. Cells may be expanded, treated, and/or genetically modified in vitro prior to administration to the subject. For example, a NELL1 polypeptide or nucleic acid molecule encoding the same can be formulated or delivered in combination (simultaneously or sequentially) with other biomolecules and/or adult stem cells, (naturally extracted and expanded or engineered; autologous, allogeneic, or xenogeneic), such as mesenchymal stem cells or immature heart cells, to create complex regenerative mixtures or cocktails that are injected, implanted or infused for systemic release into a subject. Treatments may comprise the administration of complex regenerative mixtures or cocktails that can be injected, implanted, infused or otherwise administered to the subject. The administration of the mixture or cocktail may induce systemic release of the NELL1 peptide or nucleic acid molecule encoding the same into the subject or may deliver NELL1 to a local region (e.g., local cells, local tissue, or local region or body part).

NELL1 can be added to formulations or (or used along with) products that are acellular extracellular matrix materials either extracted from natural sources (e.g. linings of urinary bladder, small intestinal submucosa, decellularized tissue from the subject, an allograft, or a xenograft, etc.) or manufactured as a synthetic. Acellular products for regenerative medicine that contain extracellular matrix material may not have all the needed signals for tissue regeneration and the addition of NELL1 can enhance the ability of some of these materials to effect cell differentiation and tissue maturation. The NELL1 polypeptide or nucleic acid molecule encoding the same may be impregnated, linked (e.g., covalently conjugated or non-covalently associated with), infused, integrated, or otherwise coupled with synthetic and/or natural matrix/scaffold materials that are administered by implantation into the body. The matrix/scaffold material may include synthetic and/or natural polymers, including but not limited to chitosan, agarose, alginate, gelatin, collagen, hyaluronic acid, fibrinogen, fibronectin, myoglobin, hemoglobin, polyethyelene glycol (PEG), polylactic acid (PLA), poly(lactic-co-glycolic acid) (PLGA), polycaprolactone, silk fibroin, ethylene vinyl acetate copolymer, etc. In some embodiments, the matrix or scaffold material may be slowly degraded to release components into the blood, thoracic or gastric cavity to promote new tissue formation. In various implementations, one or more active ingredients may be released upon degradation/dissolution of the matrix/scaffold materials (e.g., physiological degradation such as enzymatic degradation and/or hydrolysis), upon breaking covalent linkages to the matrix/scaffold material, and/or upon diffusion form the matrix scaffold material. In some embodiments, the administered treatment may comprise both acellular matrix/scaffold material as well as cells, as described above. In various implementations, the cells may be genetically modified and/or transfected (e.g., may be modified to incorporate a vector such as a plasmid) to express nucleic acids encoding the NELL1 peptide.

In practicing combination therapy, the NELL1 polypeptide or nucleic acid molecule encoding the same and the additional treatment or therapeutic agent may be administered to the subject simultaneously, either in a single composition, or as two or more distinct compositions using the same or different administration routes. Alternatively, the NELL1 polypeptide or nucleic acid molecule encoding the same may precede, or follow, the additional treatment or therapeutic agent by, e.g., intervals ranging from minutes to weeks. In at least one embodiment, the NELL1 polypeptide or nucleic acid molecule encoding the same and the additional treatment or therapeutic agent are administered within about 5 minutes to about two weeks of each other. In yet other embodiments, several days (2, 3, 4, 5, 6 or 7), several weeks (1, 2, 3, 4, 5, 6, 7 or 8) or several months (1, 2, 3, 4, 5, 6, 7 or 8) may lapse between administration of the NELL1 polypeptide or nucleic acid molecule encoding the same and the additional treatment or therapeutic agent.

### IV. Miscellaneous

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In addition, ranges provided in the specification and appended claims include both end points and all points between the end points. Therefore, a range of 2.0 to 3.0 includes 2.0, 3.0, and all points between 2.0 and 3.0.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

General methods in molecular genetics and genetic engineering useful in the present invention are described in the current editions of Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). Reagents, cloning vectors, and kits for genetic manipulation are available from commercial vendors such as BioRad, Stratagene, Invitrogen, ClonTech and Sigma-Aldrich Co.

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference, regardless of whether the phrase “incorporated by reference” is or is not used in relation to the particular reference. The foregoing detailed description and the examples that follow have been given for clarity of understanding. No unnecessary limitations are to be understood therefrom. The invention is not limited to the exact details shown and described. Variations obvious to one skilled in the art are included in the invention defined by the claims. Any section headings used herein are for organizational purposes and are not to be construed as limiting the subject matter described.

### V. Sequence Summary and Sequences

The following Table 1 provides a summary of the included sequences.

TABLE 1
Nucleotide and amino acid sequences disclosed herein.

| SEQ ID | |
|---|---|
| NO. | Description |
| 1 | Homo sapiens NELL1 isoform 1 transcript variant (nucleotide) |
| 2 | Homo sapiens NELL1 isoform 1 (amino acid) |
| 3 | Homo sapiens NELL1 isoform 2 transcript variant (nucleotide) |
| 4 | Homo sapiens NELL1 isoform 2 (amino acid) |
| 5 | Equus caballus NELL1 isoform 1 (nucleotide) |
| 6 | Equus caballus NELL1 isoform 1 (amino acid) |
| 7 | Equus caballus NELL1 isoform 2 (nucleotide) |
| 8 | Equus caballus NELL1 isoform 2 (amino acid) |
| 9 | Mus musculus NELL1 (nucleotide) |
| 10 | Mus musculus NELL1 (amino acid) |
| 11 | Rattus norvegicus NELL1 (nucleotide) |
| 12 | Rattus norvegicus NELL1 (amino acid) |
| 13 | Felis catus NELL1 isoform 1 (amino acid) |
| 14 | Felis catus NELL1 isoform 2 (amino acid) |
| 15 | Canis lupis familiaris NELL1 (amino acid) |
| 16 | Ovis aries NELL1 (amino acid) |
| 17 | Homo sapiens NELL1 fragment (amino acid) |
| 18 | Equus caballus NELL1 fragment (amino acid) |
| 19 | Bos taurus NELL1 (amino acid) |

Homo sapiens NELL1 isoform 1 nucleotide sequence (SEQ ID NO: 1) and
translated amino acid sequence (SEQ ID NO: 2)

| | |
|---|---|
| atatgcgagc gcagcacccg gcgctgccga gccacctccc ccgccgcccg ctagcaagtt | 60 |
| tggcggctcc aagccaggcg cgcctcagga tccaggctca tttgcttcca cctagcttcg | 120 |
| gtgccccctg ctaggcgggg accctcgaga gcg atg ccg atg gat ttg att tta | 174 |
| Met Pro Met Asp Leu Ile Leu | |
| gtt gtg tgg ttc tgt gtg tgc act gcc agg aca gtg gtg ggc ttt ggg | 222 |
| Val Val Trp Phe Cys Val Cys Thr Ala Arg Thr Val Val Gly Phe Gly | |
| atg gac cct gac ctt cag atg gat atc gtc acc gag ctt gac ctt gtg | 270 |
| Met Asp Pro Asp Leu Gln Met Asp Ile Val Thr Glu Leu Asp Leu Val | |
| aac acc acc ctt gga gtt gct cag gtg tct gga atg cac aat gcc agc | 318 |
| Asn Thr Thr Leu Gly Val Ala Gln Val Ser Gly Met His Asn Ala Ser | |
| aaa gca ttt tta ttt caa gac ata gaa aga gag atc cat gca gct cct | 366 |
| Lys Ala Phe Leu Phe Gln Asp Ile Glu Arg Glu Ile His Ala Ala Pro | |
| cat gtg agt gag aaa tta att cag ctg ttc cgg aac aag agt gaa ttc | 414 |
| His Val Ser Glu Lys Leu Ile Gln Leu Phe Arg Asn Lys Ser Glu Phe | |
| acc att ttg gcc act gta cag cag aag cca tcc act tca gga gtg ata | 462 |
| Thr Ile Leu Ala Thr Val Gln Gln Lys Pro Ser Thr Ser Gly Val Ile | |
| ctg tcc att cga gaa ctg gag cac agc tat ttt gaa ctg gag agc agt | 510 |
| Leu Ser Ile Arg Glu Leu Glu His Ser Tyr Phe Glu Leu Glu Ser Ser | |
| ggc ctg agg gat gag att cgg tat cac tac ata cac aat ggg aag cca | 558 |
| Gly Leu Arg Asp Glu Ile Arg Tyr His Tyr Ile His Asn Gly Lys Pro | |
| agg aca gag gca ctt cct tac cgc atg gca gat gga caa tgg cac aag | 606 |
| Arg Thr Glu Ala Leu Pro Tyr Arg Met Ala Asp Gly Gln Trp His Lys | |
| gtt gca ctg tca gtt agc gcc tct cat ctc ctg ctc cat gtc gac tgt | 654 |
| Val Ala Leu Ser Val Ser Ala Ser His Leu Leu Leu His Val Asp Cys | |
| aac agg att tat gag cgt gtg ata gac cct cca gat acc aac ctt ccc | 702 |
| Asn Arg Ile Tyr Glu Arg Val Ile Asp Pro Pro Asp Thr Asn Leu Pro | |
| cca gga atc aat tta tgg ctt ggc cag cgc aac caa aag cat ggc tta | 750 |
| Pro Gly Ile Asn Leu Trp Leu Gly Gln Arg Asn Gln Lys His Gly Leu | |
| ttc aaa ggg atc atc caa gat ggg aag atc atc ttt atg ccg aat gga | 798 |
| Phe Lys Gly Ile Ile Gln Asp Gly Lys Ile Ile Phe Met Pro Asn Gly | |
| tat ata aca cag tgt cca aat cta aat cac act tgc cca acc tgc agt | 846 |
| Tyr Ile Thr Gln Cys Pro Asn Leu Asn His Thr Cys Pro Thr Cys Ser | |
| gat ttc tta agc ctg gtg caa gga ata atg gat tta caa gag ctt ttg | 894 |
| Asp Phe Leu Ser Leu Val Gln Gly Ile Met Asp Leu Gln Glu Leu Leu | |
| gcc aag atg act gca aaa cta aat tat gca gag aca aga ctt agt caa | 942 |
| Ala Lys Met Thr Ala Lys Leu Asn Tyr Ala Glu Thr Arg Leu Ser Gln | |
| ttg gaa aac tgt cat tgt gag aag act tgt caa gtg agt gga ctg ctc | 990 |
| Leu Glu Asn Cys His Cys Glu Lys Thr Cys Gln Val Ser Gly Leu Leu | |
| tat cga gat caa gac tct tgg gta gat ggt gac cat tgc agg aac tgc | 1038 |
| Tyr Arg Asp Gln Asp Ser Trp Val Asp Gly Asp His Cys Arg Asn Cys | |
| act tgc aaa agt ggt gcc gtg gaa tgc cga agg atg tcc tgt ccc cct | 1086 |
| Thr Cys Lys Ser Gly Ala Val Glu Cys Arg Arg Met Ser Cys Pro Pro | |
| ctc aat tgc tcc cca gac tcc ctc cca gtg cac att gct ggc cag tgc | 1134 |
| Leu Asn Cys Ser Pro Asp Ser Leu Pro Val His Ile Ala Gly Gln Cys | |
| tgt aag gtc tgc cga cca aaa tgt atc tat gga gga aaa gtt ctt gca | 1182 |
| Cys Lys Val Cys Arg Pro Lys Cys Ile Tyr Gly Gly Lys Val Leu Ala | |
| gaa ggc cag cgg att tta acc aag agc tgt cgg gaa tgc cga ggt gga | 1230 |
| Glu Gly Gln Arg Ile Leu Thr Lys Ser Cys Arg Glu Cys Arg Gly Gly | |
| gtt tta gta aaa att aca gaa atg tgt cct cct ttg aac tgc tca gaa | 1278 |
| Val Leu Val Lys Ile Thr Glu Met Cys Pro Pro Leu Asn Cys Ser Glu | |
| aag gat cac att ctt cct gag aat cag tgc tgc cgt gtc tgt aga ggt | 1326 |
| Lys Asp His Ile Leu Pro Glu Asn Gln Cys Cys Arg Val Cys Arg Gly | |
| cat aac ttt tgt gca gaa gga cct aaa tgt ggt gaa aac tca gag tgc | 1374 |
| His Asn Phe Cys Ala Glu Gly Pro Lys Cys Gly Glu Asn Ser Glu Cys | |
| aaa aac tgg aat aca aaa gct act tgt gag tgc aag agt ggt tac atc | 1422 |
| Lys Asn Trp Asn Thr Lys Ala Thr Cys Glu Cys Lys Ser Gly Tyr Ile | |
| tct gtc cag gga gac tct gcc tac tgt gaa gat att gat gag tgt gca | 1470 |
| Ser Val Gln Gly Asp Ser Ala Tyr Cys Glu Asp Ile Asp Glu Cys Ala | |
| gct aag atg cat tac tgt cat gcc aat act gtg tgt gtc aac ctt cct | 1518 |
| Ala Lys Met His Tyr Cys His Ala Asn Thr Val Cys Val Asn Leu Pro | |
| ggg tta tat cgc tgt gac tgt gtc cca gga tac att cgt gtg gat gac | 1566 |
| Gly Leu Tyr Arg Cys Asp Cys Val Pro Gly Tyr Ile Arg Val Asp Asp | |
| ttc tct tgt aca gaa cac gat gaa tgt ggc agc ggc cag cac aac tgt | 1614 |
| Phe Ser Cys Thr Glu His Asp Glu Cys Gly Ser Gly Gln His Asn Cys | |
| gat gag aat gcc atc tgc acc aac act gtc cag gga cac agc tgc acc | 1662 |
| Asp Glu Asn Ala Ile Cys Thr Asn Thr Val Gln Gly His Ser Cys Thr | |
| tgc aaa ccg ggc tac gtg ggg aac ggg acc atc tgc aga gct ttc tgt | 1710 |
| Cys Lys Pro Gly Tyr Val Gly Asn Gly Thr Ile Cys Arg Ala Phe Cys | |
| gaa gag ggc tgc aga tac ggt gga acg tgt gtg gct ccc aac aaa tgt | 1758 |
| Glu Glu Gly Cys Arg Tyr Gly Gly Thr Cys Val Ala Pro Asn Lys Cys | |
| gtc tgt cca tct gga ttc aca gga agc cac tgc gag aaa gat att gat | 1806 |
| Val Cys Pro Ser Gly Phe Thr Gly Ser His Cys Glu Lys Asp Ile Asp | |
| gaa tgt tca gag gga atc att gag tgc cac aac cat tcc cgc tgc gtt | 1854 |
| Glu Cys Ser Glu Gly Ile Ile Glu Cys His Asn His Ser Arg Cys Val | |
| aac ctg cca ggg tgg tac cac tgt gag tgc aga agc ggt ttc cat gac | 1902 |
| Asn Leu Pro Gly Trp Tyr His Cys Glu Cys Arg Ser Gly Phe His Asp | |
| gat ggg acc tat tca ctg tcc ggg gag tcc tgt att gac att gat gaa | 1950 |
| Asp Gly Thr Tyr Ser Leu Ser Gly Glu Ser Cys Ile Asp Ile Asp Glu | |
| tgt gcc tta aga act cac acc tgt tgg aac gat tct gcc tgc atc aac | 1998 |
| Cys Ala Leu Arg Thr His Thr Cys Trp Asn Asp Ser Ala Cys Ile Asn | |
| ctg gca ggg ggc ttt gac tgt ctc tgc ccc tct ggg ccc tcc tgc tct | 2046 |
| Leu Ala Gly Gly Phe Asp Cys Leu Cys Pro Ser Gly Pro Ser Cys Ser | |
| ggt gac tgt cct cat gaa ggg ggg ctg aag cac aat ggc cag gtg tgg | 2094 |
| Gly Asp Cys Pro His Glu Gly Gly Leu Lys His Asn Gly Gln Val Trp | |
| acc ttg aaa gaa gac agg tgt tct gtc tgc tcc tgc aag gat ggc aag | 2142 |
| Thr Leu Lys Glu Asp Arg Cys Ser Val Cys Ser Cys Lys Asp Gly Lys | |
| ata ttc tgc cga cgg aca gct tgt gat tgc cag aat cca agt gct gac | 2190 |
| Ile Phe Cys Arg Arg Thr Ala Cys Asp Cys Gln Asn Pro Ser Ala Asp | |
| cta ttc tgt tgc cca gaa tgt gac acc aga gtc aca agt caa tgt tta | 2238 |
| Leu Phe Cys Cys Pro Glu Cys Asp Thr Arg Val Thr Ser Gln Cys Leu | |
| gac caa aat ggt cac aag ctg tat cga agt gga gac aat tgg acc cat | 2286 |
| Asp Gln Asn Gly His Lys Leu Tyr Arg Ser Gly Asp Asn Trp Thr His | |
| agc tgt cag cag tgt cgg tgt ctg gaa gga gag gta gat tgc tgg cca | 2334 |
| Ser Cys Gln Gln Cys Arg Cys Leu Glu Gly Glu Val Asp Cys Trp Pro | |
| ctc act tgc ccc aac ttg agc tgt gag tat aca gct atc tta gaa ggg | 2382 |
| Leu Thr Cys Pro Asn Leu Ser Cys Glu Tyr Thr Ala Ile Leu Glu Gly | |
| gaa tgt tgt ccc cgc tgt gtc agt gac ccc tgc cta gct gat aac atc | 2430 |
| Glu Cys Cys Pro Arg Cys Val Ser Asp Pro Cys Leu Ala Asp Asn Ile | |
| acc tat gac atc aga aaa act tgc ctg gac agc tat ggt gtt tca cgg | 2478 |
| Thr Tyr Asp Ile Arg Lys Thr Cys Leu Asp Ser Tyr Gly Val Ser Arg | |
| ctt agt ggc tca gtg tgg acg atg gct gga tct ccc tgc aca acc tgt | 2526 |
| Leu Ser Gly Ser Val Trp Thr Met Ala Gly Ser Pro Cys Thr Thr Cys | |
| aaa tgc aag aat gga aga gtc tgt tgt tct gtg gat ttt gag tgt ctt | 2574 |
| Lys Cys Lys Asn Gly Arg Val Cys Cys Ser Val Asp Phe Glu Cys Leu | |
| caa aat aat tga agtatttaca gtggactcaa cgcagaagaa tggacgaaat | 2626 |
| Gln Asn Asn * | |
| gaccatccaa cgtgattaag gataggaatc ggtagtttgg tttttttgtt tgttttgttt | 2686 |
| ttttaaccac agataattgc caaagtttcc acctgaggac ggtgtttgga ggttgccttt | 2746 |
| tggacctacc actttgctca ttcttgctaa cctagtctag gtgacctaca gtgccgtgca | 2806 |
| tttaagtcaa tggttgttaa aagaagtttc ccgtgttgta aatcatgttt cccttatcag | 2866 |
| atcatttgca aatacattta aatgatctca tggtaaatgt tgatgtattt tttggtttat | 2926 |
| tttgtgtact aacataatag agagagactc agctcctttt atttattttg ttgatttatg | 2986 |
| gatcaaattc taaaataaag ttgcctgttg tgacttttgt cccatctact gcatacttag | 3046 |
| tgctgagatc cctgtaaaat gttttgatga aaatatgtat gtagagtcca gtcgcattat | 3106 |
| acatacattt catagtgctg aaccttctta aatgcctact cattcagctt aaacaggctg | 3166 |
| aagccaagta tgacaaagag gggaagggcc aaaaacataa tcaaagaata attttaaaga | 3226 |
| gaattcttgt ctctcttgca aaaaaaaaa | 3255 |

Homo sapiens NELL1 isoform 1 amino acid sequence (SEQ ID NO: 2)
Met Pro Met Asp Leu Ile Leu Val Val Trp Phe Cys Val Cys Thr Ala
Arg Thr Val Val Gly Phe Gly Met Asp Pro Asp Leu Gln Met Asp Ile
Val Thr Glu Leu Asp Leu Val Asn Thr Thr Leu Gly Val Ala Gln Val
Ser Gly Met His Asn Ala Ser Lys Ala Phe Leu Phe Gln Asp Ile Glu
Arg Glu Ile His Ala Ala Pro His Val Ser Glu Lys Leu Ile Gln Leu
Phe Arg Asn Lys Ser Glu Phe Thr Ile Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Leu Arg Asp Glu Ile Arg Tyr His
Tyr Ile His Asn Gly Lys Pro Arg Thr Glu Ala Leu Pro Tyr Arg Met
Ala Asp Gly Gln Trp His Lys Val Ala Leu Ser Val Ser Ala Ser His
Leu Leu Leu His Val Asp Cys Asn Arg Ile Tyr Glu Arg Val Ile Asp
Pro Pro Asp Thr Asn Leu Pro Pro Gly Ile Asn Leu Trp Leu Gly Gln
Arg Asn Gln Lys His Gly Leu Phe Lys Gly Ile Ile Gln Asp Gly Lys
Ile Ile Phe Met Pro Asn Gly Tyr Ile Thr Gln Cys Pro Asn Leu Asn
His Thr Cys Pro Thr Cys Ser Asp Phe Leu Ser Leu Val Gln Gly Ile
Met Asp Leu Gln Glu Leu Leu ala Lys Met Thr Ala Lys Leu Asn Tyr
Ala Glu Thr Arg Leu Ser Gln Leu Glu Asn Cys His Cys Glu Lys Thr
Cys Gln Val Ser Gly Leu Leu Tyr Arg Asp Gln Asp Ser Trp Val Asp
Gly Asp His Cys Arg Asn Cys Thr Cys Lys Ser Gly Ala Val Glu Cys
Arg Arg Met Ser Cys Pro Pro Leu Asn Cys Ser Pro Asp Ser Leu Pro
Val His Ile Ala Gly Gln Cys Cys Lys Val Cys Arg Pro Lys Cys Ile
Tyr Gly Gly Lys Val Leu Ala Glu Gly Gln Arg Ile Leu Thr Lys Ser
Cys Arg Glu Cys Arg Gly Gly Val Leu Val Lys Ile Thr Glu Met Cys
Pro Pro Leu Asn Cys Ser Glu Lys Asp His Ile Leu Pro Glu Asn Gln
Cys Cys Arg Val Cys Arg Gly His Asn Phe Cys Ala Glu Gly Pro Lys
Cys Gly Glu Asn Ser Glu Cys Lys Asn Trp Asn Thr Lys Ala Thr Cys
Glu Cys Lys Ser Gly Tyr Ile Ser Val Gln Gly Asp Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Pro
Gly Tyr Ile Arg Val Asp Asp Phe Ser Cys Thr Glu His Asp Glu Cys
Gly Ser Gly Gln His Asn Cys Asp Glu Asn Ala Ile Cys Thr Asn Thr
Val Gln Gly His Ser Cys Thr Cys Lys Pro Gly Tyr Val Gly Asn Gly
Thr Ile Cys Arg Ala Phe Cys Glu Glu Gly Cys Arg Tyr Gly Gly Thr
Cys Val Ala Pro Asn Lys Cys Val Cys Pro Ser Gly Phe Thr Gly Ser
His Cys Glu Lys Asp Ile Asp Glu Cys Ser Glu Gly Ile Ile Glu Cys
His Asn His Ser Arg Cys Val Asn Leu Pro Gly Trp Tyr His Cys Glu
Cys Arg Ser Gly Phe His Asp Asp Gly Thr Tyr Ser Leu Ser Gly Glu
Ser Cys Ile Asp Ile Asp Glu Cys Ala Leu Arg Thr His Thr Cys Trp
Asn Asp Ser Ala Cys Ile Asn Leu Ala Gly Gly Phe Asp Cys Leu Cys
Pro Ser Gly Pro Ser Cys Ser Gly Asp Cys Pro His Glu Gly Gly Leu
Lys His Asn Gly Gln Val Trp Thr Leu Lys Glu Asp Arg Cys Ser Val
Cys Ser Cys Lys Asp Gly Lys Ile Phe Cys Arg Arg Thr Ala Cys Asp
Cys Gln Asn Pro Ser Ala Asp Leu Phe Cys Cys Pro Glu Cys Asp Thr
Arg Val Thr Ser Gln Cys Leu Asp Gln Asn Gly His Lys Leu Tyr Arg
Ser Gly Asp Asn Trp Thr His Ser Cys Gln Gln Cys Arg Cys Leu Glu
Gly Glu Val Asp Cys Trp Pro Leu Thr Cys Pro Asn Leu Ser Cys Glu
Tyr Thr Ala Ile Leu Glu Gly Glu Cys Cys Pro Arg Cys Val Ser Asp
Pro Cys Leu Ala Asp Asn Ile Thr Tyr Asp Ile Arg Lys Thr Cys Leu
Asp Ser Tyr Gly Val Ser Arg Leu Ser Gly Ser Val Trp Thr Met Ala
Gly Ser Pro Cys Thr Thr Cys Lys Cys Lys Asn Gly Arg Val Cys Cys
Ser Val Asp Phe Glu Cys Leu Gln Asn Asn
Homo sapiens NELL1 isoform 2 nucleotide sequence (SEQ ID NO: 3) and
translated amino acid sequence (SEQ ID NO: 4)

| | |
|---|---|
| atatgcgagc gcagcacccg gcgctgccga gccacctccc ccgccgcccg ctagcaagtt | 60 |
| tggcggctcc aagccaggcg cgcctcagga tccaggctca tttgcttcca cctagcttcg | 120 |
| gtgccccctg ctaggcgggg accctcgaga gcg atg ccg atg gat ttg att tta | 174 |
| Met Pro Met Asp Leu Ile Leu | |
| gtt gtg tgg ttc tgt gtg tgc act gcc agg aca gtg gtg ggc ttt ggg | 222 |
| Val Val Trp Phe Cys Val Cys Thr Ala Arg Thr Val Val Gly Phe Gly | |
| atg gac cct gac ctt cag atg gat atc gtc acc gag ctt gac ctt gtg | 270 |
| Met Asp Pro Asp Leu Gln Met Asp Ile Val Thr Glu Leu Asp Leu Val | |
| aac acc acc ctt gga gtt gct cag gtg tct gga atg cac aat gcc agc | 318 |
| Asn Thr Thr Leu Gly Val Ala Gln Val Ser Gly Met His Asn Ala Ser | |
| aaa gca ttt tta ttt caa gac ata gaa aga gag atc cat gca gct cct | 366 |
| Lys Ala Phe Leu Phe Gln Asp Ile Glu Arg Glu Ile His Ala Ala Pro | |
| cat gtg agt gag aaa tta att cag ctg ttc cgg aac aag agt gaa ttc | 414 |
| His Val Ser Glu Lys Leu Ile Gln Leu Phe Arg Asn Lys Ser Glu Phe | |
| acc att ttg gcc act gta cag cag aag cca tcc act tca gga gtg ata | 462 |
| Thr Ile Leu Ala Thr Val Gln Gln Lys Pro Ser Thr Ser Gly Val Ile | |
| ctg tcc att cga gaa ctg gag cac agc tat ttt gaa ctg gag agc agt | 510 |
| Leu Ser Ile Arg Glu Leu Glu His Ser Tyr Phe Glu Leu Glu Ser Ser | |
| ggc ctg agg gat gag att cgg tat cac tac ata cac aat ggg aag cca | 558 |
| Gly Leu Arg Asp Glu Ile Arg Tyr His Tyr Ile His Asn Gly Lys Pro | |
| agg aca gag gca ctt cct tac cgc atg gca gat gga caa tgg cac aag | 606 |
| Arg Thr Glu Ala Leu Pro Tyr Arg Met Ala Asp Gly Gln Trp His Lys | |
| gtt gca ctg tca gtt agc gcc tct cat ctc ctg ctc cat gtc gac tgt | 654 |
| Val Ala Leu Ser Val Ser Ala Ser His Leu Leu Leu His Val Asp Cys | |
| aac agg att tat gag cgt gtg ata gac cct cca gat acc aac ctt ccc | 702 |
| Asn Arg Ile Tyr Glu Arg Val Ile Asp Pro Pro Asp Thr Asn Leu Pro | |
| cca gga atc aat tta tgg ctt ggc cag cgc aac caa aag cat ggc tta | 750 |
| Pro Gly Ile Asn Leu Trp Leu Gly Gln Arg Asn Gln Lys His Gly Leu | |
| ttc aaa ggg atc atc caa gat ggg aag atc atc ttt atg ccg aat gga | 798 |
| Phe Lys Gly Ile Ile Gln Asp Gly Lys Ile Ile Phe Met Pro Asn Gly | |
| tat ata aca cag tgt cca aat cta aat cac act tgc cca acc tgc agt | 846 |
| Tyr Ile Thr Gln Cys Pro Asn Leu Asn His Thr Cys Pro Thr Cys Ser | |
| gat ttc tta agc ctg gtg caa gga ata atg gat tta caa gag ctt ttg | 894 |
| Asp Phe Leu Ser Leu Val Gln Gly Ile Met Asp Leu Gln Glu Leu Leu | |
| gcc aag atg act gca aaa cta aat tat gca gag aca aga ctt agt caa | 942 |
| Ala Lys Met Thr Ala Lys Leu Asn Tyr Ala Glu Thr Arg Leu Ser Gln | |
| ttg gaa aac tgt cat tgt gag aag act tgt caa gtg agt gga ctg ctc | 990 |
| Leu Glu Asn Cys His Cys Glu Lys Thr Cys Gln Val Ser Gly Leu Leu | |
| tat cga gat caa gac tct tgg gta gat ggt gac cat tgc agg aac tgc | 1038 |
| Tyr Arg Asp Gln Asp Ser Trp Val Asp Gly Asp His Cys Arg Asn Cys | |
| act tgc aaa agt ggt gcc gtg gaa tgc cga agg atg tcc tgt ccc cct | 1086 |
| Thr Cys Lys Ser Gly Ala Val Glu Cys Arg Arg Met Ser Cys Pro Pro | |
| ctc aat tgc tcc cca gac tcc ctc cca gtg cac att gct ggc cag tgc | 1134 |
| Leu Asn Cys Ser Pro Asp Ser Leu Pro Val His Ile Ala Gly Gln Cys | |
| tgt aag gtc tgc cga cca aaa tgt atc tat gga gga aaa gtt ctt gca | 1182 |
| Cys Lys Val Cys Arg Pro Lys Cys Ile Tyr Gly Gly Lys Val Leu Ala | |
| gaa ggc cag cgg att tta acc aag agc tgt cgg gaa tgc cga ggt gga | 1230 |
| Glu Gly Gln Arg Ile Leu Thr Lys Ser Cys Arg Glu Cys Arg Gly Gly | |
| gtt tta gta aaa att aca gaa atg tgt cct cct ttg aac tgc tca gaa | 1278 |
| Val Leu Val Lys Ile Thr Glu Met Cys Pro Pro Leu Asn Cys Ser Glu | |
| aag gat cac att ctt cct gag aat cag tgc tgc cgt gtc tgt aga ggt | 1326 |
| Lys Asp His Ile Leu Pro Glu Asn Gln Cys Cys Arg Val Cys Arg Gly | |
| cat aac ttt tgt gca gaa gga cct aaa tgt ggt gaa aac tca gag tgc | 1374 |
| His Asn Phe Cys Ala Glu Gly Pro Lys Cys Gly Glu Asn Ser Glu Cys | |
| aaa aac tgg aat aca aaa gct act tgt gag tgc aag agt ggt tac atc | 1422 |
| Lys Asn Trp Asn Thr Lys Ala Thr Cys Glu Cys Lys Ser Gly Tyr Ile | |
| tct gtc cag gga gac tct gcc tac tgt gaa gat att gat gag tgt gca | 1470 |
| Ser Val Gln Gly Asp Ser Ala Tyr Cys Glu Asp Ile Asp Glu Cys Ala | |
| gct aag atg cat tac tgt cat gcc aat act gtg tgt gtc aac ctt cct | 1518 |
| Ala Lys Met His Tyr Cys His Ala Asn Thr Val Cys Val Asn Leu Pro | |
| ggg tta tat cgc tgt gac tgt gtc cca gga tac att cgt gtg gat gac | 1566 |
| Gly Leu Tyr Arg Cys Asp Cys Val Pro Gly Tyr Ile Arg Val Asp Asp | |
| ttc tct tgt aca gaa cac gat gaa tgt ggc agc ggc cag cac aac tgt | 1614 |
| Phe Ser Cys Thr Glu His Asp Glu Cys Gly Ser Gly Gln His Asn Cys | |
| gat gag aat gcc atc tgc acc aac act gtc cag gga cac agc tgc acc | 1662 |
| Asp Glu Asn Ala Ile Cys Thr Asn Thr Val Gln Gly His Ser Cys Thr | |
| tgc aaa ccg ggc tac gtg ggg aac ggg acc atc tgc aga gct ttc tgt | 1710 |
| Cys Lys Pro Gly Tyr Val Gly Asn Gly Thr Ile Cys Arg Ala Phe Cys | |
| gaa gag ggc tgc aga tac ggt gga acg tgt gtg gct ccc aac aaa tgt | 1758 |
| Glu Glu Gly Cys Arg Tyr Gly Gly Thr Cys Val Ala Pro Asn Lys Cys | |
| gtc tgt cca tct gga ttc aca gga agc cac tgc gag aaa gac att gat | 1806 |
| Val Cys Pro Ser Gly Phe Thr Gly Ser His Cys Glu Lys Asp Ile Asp | |
| gaa tgt gcc tta aga act cac acc tgt tgg aac gat tct gcc tgc atc | 1854 |
| Glu Cys Ala Leu Arg Thr His Thr Cys Trp Asn Asp Ser Ala Cys Ile | |
| aac ctg gca ggg ggc ttt gac tgt ctc tgc ccc tct ggg ccc tcc tgc | 1902 |
| Asn Leu Ala Gly Gly Phe Asp Cys Leu Cys Pro Ser Gly Pro Ser Cys | |
| tct ggt gac tgt cct cat gaa ggg ggg ctg aag cac aat ggc cag gtg | 1950 |
| Ser Gly Asp Cys Pro His Glu Gly Gly Leu Lys His Asn Gly Gln Val | |
| tgg acc ttg aaa gaa gac agg tgt tct gtc tgc tcc tgc aag gat ggc | 1998 |
| Trp Thr Leu Lys Glu Asp Arg Cys Ser Val Cys Ser Cys Lys Asp Gly | |
| aag ata ttc tgc cga cgg aca gct tgt gat tgc cag aat cca agt gct | 2046 |
| Lys Ile Phe Cys Arg Arg Thr Ala Cys Asp Cys Gln Asn Pro Ser Ala | |
| gac cta ttc tgt tgc cca gaa tgt gac acc aga gtc aca agt caa tgt | 2094 |
| Asp Leu Phe Cys Cys Pro Glu Cys Asp Thr Arg Val Thr Ser Gln Cys | |
| tta gac caa aat ggt cac aag ctg tat cga agt gga gac aat tgg acc | 2142 |
| Leu Asp Gln Asn Gly His Lys Leu Tyr Arg Ser Gly Asp Asn Trp Thr | |
| cat agc tgt cag cag tgt cgg tgt ctg gaa gga gag gta gat tgc tgg | 2190 |
| His Ser Cys Gln Gln Cys Arg Cys Leu Glu Gly Glu Val Asp Cys Trp | |
| cca ctc act tgc ccc aac ttg agc tgt gag tat aca gct atc tta gaa | 2238 |
| Pro Leu Thr Cys Pro Asn Leu Ser Cys Glu Tyr Thr Ala Ile Leu Glu | |
| ggg gaa tgt tgt ccc cgc tgt gtc agt gac ccc tgc cta gct gat aac | 2286 |
| Gly Glu Cys Cys Pro Arg Cys Val Ser Asp Pro Cys Leu Ala Asp Asn | |
| atc acc tat gac atc aga aaa act tgc ctg gac agc tat ggt gtt tca | 2334 |
| Ile Thr Tyr Asp Ile Arg Lys Thr Cys Leu Asp Ser Tyr Gly Val Ser | |
| cgg ctt agt ggc tca gtg tgg acg atg gct gga tct ccc tgc aca acc | 2382 |
| Arg Leu Ser Gly Ser Val Trp Thr Met Ala Gly Ser Pro Cys Thr Thr | |
| tgt aaa tgc aag aat gga aga gtc tgt tgt tct gtg gat ttt gag tgt | 2430 |
| Cys Lys Cys Lys Asn Gly Arg Val Cys Cys Ser Val Asp Phe Glu Cys | |
| ctt caa aat aat tga agtatttaca gtggactcaa cgcagaagaa tggacgaaat | 2485 |
| Leu Gln Asn Asn * | |
| gaccatccaa cgtgattaag gataggaatc ggtagtttgg tttttttgtt tgttttgttt | 2545 |
| ttttaaccac agataattgc caaagtttcc acctgaggac ggtgtttgga ggttgccttt | 2605 |
| tggacctacc actttgctca ttcttgctaa cctagtctag gtgacctaca gtgccgtgca | 2665 |
| tttaagtcaa tggttgttaa aagaagtttc ccgtgttgta aatcatgttt cccttatcag | 2725 |
| atcatttgca aatacattta aatgatctca tggtaaatgt tgatgtattt tttggtttat | 2785 |
| tttgtgtact aacataatag agagagactc agctcctttt atttattttg ttgatttatg | 2845 |
| gatcaaattc taaaataaag ttgcctgttg tgacttttgt cccatctact gcatacttag | 2905 |
| tgctgagatc cctgtaaaat gttttgatga aaatatgtat gtagagtcca gtcgcattat | 2965 |
| acatacattt catagtgctg aaccttctta aatgcctact cattcagctt aaacaggctg | 3025 |
| aagccaagta tgacaaagag gggaagggcc aaaaacataa tcaaagaata attttaaaga | 3085 |
| gaattcttgt ctctcttgca aaaaaaaaa | 3114 |

Homo sapiens NELL1 isoform 2 amino acid sequence (SEQ ID NO: 4)
Met Pro Met Asp Leu Ile Leu Val Val Trp Phe Cys Val Cys Thr Ala
Arg Thr Val Val Gly Phe Gly Met Asp Pro Asp Leu Gln Met Asp Ile
Val Thr Glu Leu Asp Leu Val Asn Thr Thr Leu Gly Val Ala Gln Val
Ser Gly Met His Asn Ala Ser Lys Ala Phe Leu Phe Gln Asp Ile Glu
Arg Glu Ile His Ala Ala Pro His Val Ser Glu Lys Leu Ile Gln Leu
Phe Arg Asn Lys Ser Glu Phe Thr Ile Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Leu Arg Asp Glu Ile Arg Tyr His
Tyr Ile His Asn Gly Lys Pro Arg Thr Glu Ala Leu Pro Tyr Arg Met
Ala Asp Gly Gln Trp His Lys Val Ala Leu Ser Val Ser Ala Ser His
Leu Leu Leu His Val Asp Cys Asn Arg Ile Tyr Glu Arg Val Ile Asp
Pro Pro Asp Thr Asn Leu Pro Pro Gly Ile Asn Leu Trp Leu Gly Gln
Arg Asn Gln Lys His Gly Leu Phe Lys Gly Ile Ile Gln Asp Gly Lys
Ile Ile Phe Met Pro Asn Gly Tyr Ile Thr Gln Cys Pro Asn Leu Asn
His Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Ser Gln Leu Glu Asn Cys His Cys Glu Lys Thr
Cys Gln Val Ser Gly Leu Leu Tyr Arg Asp Gln Asp Ser Trp Val Asp
Gly Asp His Cys Arg Asn Cys Thr Cys Lys Ser Gly Ala Val Glu Cys
Arg Arg Met Ser Cys Pro Pro Leu Asn Cys Ser Pro Asp Ser Leu Pro
Val His Ile Ala Gly Gln Cys Cys Lys Val Cys Arg Pro Lys Cys Ile
Tyr Gly Gly Lys Val Leu Ala Glu Gly Gln Arg Ile Leu Thr Lys Ser
Cys Arg Glu Cys Arg Gly Gly Val Leu Val Lys Ile Thr Glu Met Cys
Pro Pro Leu Asn Cys Ser Glu Lys Asp His Ile Leu Pro Glu Asn Gln
Cys Cys Arg Val Cys Arg Gly His Asn Phe Cys Ala Glu Gly Pro Lys
Cys Gly Glu Asn Ser Glu Cys Lys Asn Trp Asn Thr Lys Ala Thr Cys
Glu Cys Lys Ser Gly Tyr Ile Ser Val Gln Gly Asp Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Pro
Gly Tyr Ile Arg Val Asp Asp Phe Ser Cys Thr Glu His Asp Glu Cys
Gly Ser Gly Gln His Asn Cys Asp Glu Asn Ala Ile Cys Thr Asn Thr
Val Gln Gly His Ser Cys Thr Cys Lys Pro Gly Tyr Val Gly Asn Gly
Thr Ile Cys Arg Ala Phe Cys Glu Glu Gly Cys Arg Tyr Gly Gly Thr
Cys Val Ala Pro Asn Lys Cys Val Cys Pro Ser Gly Phe Thr Gly Ser
His Cys Glu Lys Asp Ile Asp Glu Cys Ala Leu Arg Thr His Thr Cys
Trp Asn Asp Ser Ala Cys Ile Asn Leu Ala Gly Gly Phe Asp Cys Leu
Cys Pro Ser Gly Pro Ser Cys Ser Gly Asp Cys Pro His Glu Gly Gly
Leu Lys His Asn Gly Gln Val Trp Thr Leu Lys Glu Asp Arg Cys Ser
Val Cys Ser Cys Lys Asp Gly Lys Ile Phe Cys Arg Arg Thr Ala Cys
Asp Cys Gln Asn Pro Ser Ala Asp Leu Phe Cys Cys Pro Glu Cys Asp
Thr Arg Val Thr Ser Gln Cys Leu Asp Gln Asn Gly His Lys Leu Tyr
Arg Ser Gly Asp Asn Trp Thr His Ser Cys Gln Gln Cys Arg Cys Leu
Glu Gly Glu Val Asp Cys Trp Pro Leu Thr Cys Pro Asn Leu Ser Cys
Glu Tyr Thr Ala Ile Leu Glu Gly Glu Cys Cys Pro Arg Cys Val Ser
Asp Pro Cys Leu Ala Asp Asn Ile Thr Tyr Asp Ile Arg Lys Thr Cys
Leu Asp Ser Tyr Gly Val Ser Arg Leu Ser Gly Ser Val Trp Thr Met
Ala Gly Ser Pro Cys Thr Thr Cys Lys Cys Lys Asn Gly Arg Val Cys
Cys Ser Val Asp Phe Glu Cys Leu Gln Asn Asn
Equus caballus NELL1 isoform 1 nucleotide sequence (SEQ ID NO: 5) and
translated amino acid sequence (SEQ ID NO: 6)

| | |
|---|---|
| atg ggc ttt ggg atg gac ccc gac ctt caa atg gat att atc acc gag | 48 |
| Met Gly Phe Gly Met Asp Pro Asp Leu Gln Met Asp Ile Ile Thr Glu | |
| ctc gac ctc gtg aac acc acc ctt gga gtc act cag gtg tcc gga ctg | 96 |
| Leu Asp Leu Val Asn Thr Thr Leu Gly Val Thr Gln Val Ser Gly Leu | |
| cac aat gcc agc aaa gca ttt tta ttt caa gat gta gag aga gag atc | 144 |
| His Asn Ala Ser Lys Ala Phe Leu Phe Gln Asp Val Glu Arg Glu Ile | |
| cat gca gcc cca cac gtg agt gag aaa tta att cag ctg ttc cgg aat | 192 |
| His Ala Ala Pro His Val Ser Glu Lys Leu Ile Gln Leu Phe Arg Asn | |
| aag agt gaa ttc acc ttt ttg gcc act gtg cag cag aag ccg tca act | 240 |
| Lys Ser Glu Phe Thr Phe Leu Ala Thr Val Gln Gln Lys Pro Ser Thr | |
| tca gga gtg ata ctg tcc att cga gaa ctg gaa aac agt tat ttt gaa | 288 |
| Ser Gly Val Ile Leu Ser Ile Arg Glu Leu Glu Asn Ser Tyr Phe Glu | |
| ctg gag agc agt ggc ctg aga gat gag att cga tat cac tac aca cac | 336 |
| Leu Glu Ser Ser Gly Leu Arg Asp Glu Ile Arg Tyr His Tyr Thr His | |
| aag ggg aag ccc agg aca gag gca ctt ccc tac cgg atg gcg gac gga | 384 |
| Lys Gly Lys Pro Arg Thr Glu Ala Leu Pro Tyr Arg Met Ala Asp Gly | |
| cgg tgg cac aag gtg gcg ctg tca gtt agc gcc tct cat ctc ctg ctc | 432 |
| Arg Trp His Lys Val Ala Leu Ser Val Ser Ala Ser His Leu Leu Leu | |
| cac atc gac tgc aac agg att tat gaa cgt gtg ata gac act cct gag | 480 |
| His Ile Asp Cys Asn Arg Ile Tyr Glu Arg Val Ile Asp Thr Pro Glu | |
| acc aac ctc ccc cca gga agc aat ttg tgg ctg ggt cag cga aac caa | 528 |
| Thr Asn Leu Pro Pro Gly Ser Asn Leu Trp Leu Gly Gln Arg Asn Gln | |
| aag cac ggc tta ttc aaa gga atc atc caa gat gga aaa atc atc ttc | 576 |
| Lys His Gly Leu Phe Lys Gly Ile Ile Gln Asp Gly Lys Ile Ile Phe | |
| atg ccg aat gga tac ata aca cag tgt ccg aac ctg aat cgc act tgc | 624 |
| Met Pro Asn Gly Tyr Ile Thr Gln Cys Pro Asn Leu Asn Arg Thr Cys | |
| cca acg tgc agt gat ttc tta agc ctg gtg caa gga atc atg gat tta | 672 |
| Pro Thr Cys Ser Asp Phe Leu Ser Leu Val Gln Gly Ile Met Asp Leu | |
| caa gag ctt ctg gcc aag atg act gcg aaa cta aat tat gca gag aca | 720 |
| Gln Glu Leu Leu Ala Lys Met Thr Ala Lys Leu Asn Tyr Ala Glu Thr | |
| cga ctt agt caa ttg gaa aac tgc cac tgc gag aag acc tgt caa gtg | 768 |
| Arg Leu Ser Gln Leu Glu Asn Cys His Cys Glu Lys Thr Cys Gln Val | |
| agt gga ctg ctc tat aga gac cag gac tcc tgg gtt gat ggc gat cac | 816 |
| Ser Gly Leu Leu Tyr Arg Asp Gln Asp Ser Trp Val Asp Gly Asp His | |
| tgt agg aac tgc acg tgc aaa agc ggc gct gtg gaa tgt cgg agg atg | 864 |
| Cys Arg Asn Cys Thr Cys Lys Ser Gly Ala Val Glu Cys Arg Arg Met | |
| tct tgt ccc cct ctc aat tgc tcc cca gac tcc ctc cct gtg cac gtt | 912 |
| Ser Cys Pro Pro Leu Asn Cys Ser Pro Asp Ser Leu Pro Val His Val | |
| gcc ggc cag tgc tgt aag gtc tgc cga cca aaa tgt atc tac gga ggg | 960 |
| Ala Gly Gln Cys Cys Lys Val Cys Arg Pro Lys Cys Ile Tyr Gly Gly | |
| aaa gtc ctt gca gaa ggc cag cgg att tta acc aag agc tgt cgg gaa | 1008 |
| Lys Val Leu Ala Glu Gly Gln Arg Ile Leu Thr Lys Ser Cys Arg Glu | |
| tgc cga ggt gga gtt tta gtg aaa att aca gaa gcg tgc cct cct ttg | 1056 |
| Cys Arg Gly Gly Val Leu Val Lys Ile Thr Glu Ala Cys Pro Pro Leu | |
| aac tgc tca gac aag gat cac att ctc cca gag aat cag tgc tgc agc | 1104 |
| Asn Cys Ser Asp Lys Asp His Ile Leu Pro Glu Asn Gln Cys Cys Ser | |
| gtc tgc aga ggt cat aac ttt tgt gcg gaa gga cct aaa tgt ggt gaa | 1152 |
| Val Cys Arg Gly His Asn Phe Cys Ala Glu Gly Pro Lys Cys Gly Glu | |
| aat tca gag tgc aaa aac tgg aat aca aaa gct act tgc gag tgc aag | 1200 |
| Asn Ser Glu Cys Lys Asn Trp Asn Thr Lys Ala Thr Cys Glu Cys Lys | |
| aat ggt tat atc tct gtc cag ggg gac tcc gcc tac tgt gaa gat atc | 1248 |
| Asn Gly Tyr Ile Ser Val Gln Gly Asp Ser Ala Tyr Cys Glu Asp Ile | |
| gat gag tgt gct gct aag atg cat tac tgt cgt gcc aat act gtg tgt | 1296 |
| Asp Glu Cys Ala Ala Lys Met His Tyr Cys Arg Ala Asn Thr Val Cys | |
| gtc aac ctg cct ggg tta tat cgg tgt gac tgt gtc ccg gga tac att | 1344 |
| Val Asn Leu Pro Gly Leu Tyr Arg Cys Asp Cys Val Pro Gly Tyr Ile | |
| cgc gtg gat gat ttc tct tgt aca gaa cat gac gaa tgt ggc agc ggg | 1392 |
| Arg Val Asp Asp Phe Ser Cys Thr Glu His Asp Glu Cys Gly Ser Gly | |
| cag cac aac tgt gat gag aat gcc atc tgc acc aac act gtc cag gga | 1440 |
| Gln His Asn Cys Asp Glu Asn Ala Ile Cys Thr Asn Thr Val Gln Gly | |
| cac agc tgc acc tgc aaa ccg ggc tac gtg ggg aat ggg acc agc tgc | 1488 |
| His Ser Cys Thr Cys Lys Pro Gly Tyr Val Gly Asn Gly Thr Ser Cys | |
| aga gcg ttc tgc gaa gag ggc tgc aga tat ggc ggg aca tgc gtg gct | 1536 |
| Arg Ala Phe Cys Glu Glu Gly Cys Arg Tyr Gly Gly Thr Cys Val Ala | |
| cct aac aaa tgt gtc tgt cct tct gga ttc aca gga agc cac tgt gag | 1584 |
| Pro Asn Lys Cys Val Cys Pro Ser Gly Phe Thr Gly Ser His Cys Glu | |
| aaa gat att gat gaa tgt aca gag gga atc att gag tgc cac aac cat | 1632 |
| Lys Asp Ile Asp Glu Cys Thr Glu Gly Ile Ile Glu Cys His Asn His | |
| tcc cgc tgc gtt aac ctg cca ggg tgg tac cac tgt gag tgc aga agc | 1680 |
| Ser Arg Cys Val Asn Leu Pro Gly Trp Tyr His Cys Glu Cys Arg Ser | |
| ggt ttc cat gac gat ggg acc tat tca ctg tcc ggg gag tcc tgt att | 1728 |
| Gly Phe His Asp Asp Gly Thr Tyr Ser Leu Ser Gly Glu Ser Cys Ile | |
| gac att gat gaa tgt gcc tta aga act cac acc tgt tgg aat gat tct | 1776 |
| Asp Ile Asp Glu Cys Ala Leu Arg Thr His Thr Cys Trp Asn Asp Ser | |
| gcc tgc atc aac ttg gca ggg ggc ttc gac tgc ctg tgt ccc tca ggg | 1824 |
| Ala Cys Ile Asn Leu Ala Gly Gly Phe Asp Cys Leu Cys Pro Ser Gly | |
| cca tcc tgc tct ggt gac tgc ccc cac gaa gga gga ctg aag cgc aac | 1872 |
| Pro Ser Cys Ser Gly Asp Cys Pro His Glu Gly Gly Leu Lys Arg Asn | |
| ggg cag gtg tgg acc ctg aaa gaa gac agg tgt tct gtg tgt tcc tgc | 1920 |
| Gly Gln Val Trp Thr Leu Lys Glu Asp Arg Cys Ser Val Cys Ser Cys | |
| aag gat ggg aag ata ttc tgc cga cgg aca gct tgt gat tgc cag aat | 1968 |
| Lys Asp Gly Lys Ile Phe Cys Arg Arg Thr Ala Cys Asp Cys Gln Asn | |
| cca agc gtt gac ctt ttc tgt tgc cca gag tgt gac acc agg gtc aca | 2016 |
| Pro Ser Val Asp Leu Phe Cys Cys Pro Glu Cys Asp Thr Arg Val Thr | |
| agt caa tgt tta gac caa aat gga cac aag ctc tat cga agt gga gac | 2064 |
| Ser Gln Cys Leu Asp Gln Asn Gly His Lys Leu Tyr Arg Ser Gly Asp | |
| aat tgg act cac agc tgt cag cag tgc cgg tgt ctg gaa gga gag gta | 2112 |
| Asn Trp Thr His Ser Cys Gln Gln Cys Arg Cys Leu Glu Gly Glu Val | |
| gat tgc tgg cca ctc act tgc ccc aga ttg agc tgt gag tac aca gcc | 2160 |
| Asp Cys Trp Pro Leu Thr Cys Pro Arg Leu Ser Cys Glu Tyr Thr Ala | |
| atc ttg gaa ggg gag tgt tgt cca cgc tgt gtc agc gac ccc tgc ctg | 2208 |
| Ile Leu Glu Gly Glu Cys Cys Pro Arg Cys Val Ser Asp Pro Cys Leu | |
| gcg gat aac atc gtc tat gac atc aga gaa act tgc ctg gac agc tat | 2256 |
| Ala Asp Asn Ile Val Tyr Asp Ile Arg Glu Thr Cys Leu Asp Ser Tyr | |
| gga gtt tca agg ctt agt ggc tca gtg tgg aca ttg gct gga tct ccc | 2304 |
| Gly Val Ser Arg Leu Ser Gly Ser Val Trp Thr Leu Ala Gly Ser Pro | |
| tgc acg acc tgc aaa tgc aag aat gga agt gtc tgc tgt tct gtg gat | 2352 |
| Cys Thr Thr Cys Lys Cys Lys Asn Gly Ser Val Cys Cys Ser Val Asp | |
| ttg gag tgt ctt cat aat aat tga aggatttaaa atggactcat gatcgccaga | 2406 |
| Leu Glu Cys Leu His Asn Asn * | |
| gaaaaatgga caaatgacca tccatgatga tgaaagaaca ggagttggtg ttttttttac | 2466 |
| cacagacaat taccaaagtc tccgtctgag gaaggtgttt gcaggttgcc ttttggacct | 2526 |
| cccactctgc tcattcttgc taacctagtc taggtgacct acagtgcatt tcagtctatg | 2586 |
| gttgttaaaa gaagttttcc gtgttgtaaa tcacgtttcc cttaccaggt cattgcaaat | 2646 |
| acatttaaat gatttcatgg taaatgttga tgtatttttt gggtttattt tgtgtactaa | 2706 |
| cataatagag attcagctgc ttttatttat ttttttcttg acttttggat caaattcaac | 2766 |
| aaataaagtt gcctgttgtg atttt | 2791 |

Equus caballus NELL1 isoform 1 amino acid sequence (SEQ ID NO: 6)
Met Gly Phe Gly Met Asp Pro Asp Leu Gln Met Asp Ile Ile Thr Glu
Leu Asp Leu Val Asn Thr Thr Leu gly Val Thr Gln Val Ser Gly Leu
His Asn Ala Ser Lys Ala Phe Leu Phe Gln Asp Val Glu Arg Glu Ile
His Ala Ala Pro His Val Ser Glu Lys Leu Ile Gln Leu Phe Arg Asn
Lys Ser Glu Phe Thr Phe Leu Ala Thr Val Gln Gln Lys Pro Ser Thr
Ser Gly Val Ile Leu Ser Ile Arg Glu Leu Glu Asn Ser Tyr Phe Glu
Leu Glu Ser Ser Gly Leu Arg Asp Glu Ile Arg Tyr His Tyr Thr His
Lys Gly Lys Pro Arg Thr Glu Ala Leu Pro Tyr Arg Met Ala Asp Gly
Arg Trp His Lys Val Ala Leu Ser Val Ser Ala Ser His Leu Leu Leu
His Ile Asp Cys Asn Arg Ile Tyr Glu Arg Val Ile Asp Thr Pro Glu
Thr Asn Leu Pro Pro Gly Ser Asn Leu Trp Leu Gly Gln Arg Asn Gln
Lys His Gly Leu Phe Lys Gly Ile Ile Gln Asp Gly Lys Ile Ile Phe
Met Pro Asn Gly Tyr Ile Thr Gln Cys Pro Asn Leu Asn Arg Thr Cys
Pro Thr Cys Ser Asp Phe Leu Ser Leu Val Gln Gly Ile Met Asp Leu
Gln Glu Leu Leu Ala Lys Met Thr Ala Lys Leu Asn Tyr Ala Glu Thr
Arg Leu Ser Gln Leu Glu Asn Cys His Cys Glu Lys Thr Cys Gln Val
Ser Gly Leu Leu Tyr Arg Asp Gln Asp Ser Trp Val Asp Gly Asp His
Cys Arg Asn Cys Thr Cys Lys Ser Gly Ala Val Glu Cys Arg Arg Met
Ser Cys Pro Pro Leu Asn Cys Ser Pro Asp Ser Leu Pro Val His Val
Ala Gly Gln Cys Cys Lys Val Cys Arg Pro Lys Cys Ile Tyr Gly Gly
Lys Val Leu Ala Glu Gly Gln Arg Ile Leu Thr Lys Ser Cys Arg Glu
Cys Arg Gly Gly Val Leu Val Lys Ile Thr Glu Ala Cys Pro Pro Leu
Asn Cys Ser Asp Lys Asp His Ile Leu Pro Glu Asn Gln Cys Cys Ser
Val Cys Arg Gly His Asn Phe Cys Ala Glu Gly Pro Lys Cys Gly Glu
Asn Ser Glu Cys Lys Asn Trp Asn Thr Lys Ala Thr Cys Glu Cys Lys
Asn Gly Tyr Ile Ser Val Gln Gly Asp Ser Ala Tyr Cys Glu Asp Ile
Asp Glu Cys Ala Ala Lys Met His Tyr Cys Arg Ala Asn Thr Val Cys
Val Asn Leu Pro Gly Leu Tyr Arg Cys Asp Cys Val Pro Gly Tyr Ile
Arg Val Asp Asp Phe Ser Cys Thr Glu His Asp Glu Cys Gly Ser Gly
Gln His Asn Cys Asp Glu Asn Ala Ile Cys Thr Asn Thr Val Gln Gly
His Ser Cys Thr Cys Lys Pro Gly Tyr Val Gly Asn Gly Thr Ser Cys
Arg Ala Phe Cys Glu Glu Gly Cys Arg Tyr Gly Gly Thr Cys Val Ala
Pro Asn Lys Cys Val Cys Pro Ser Gly Phe Thr Gly Ser His Cys Glu
Lys Asp Ile Asp Glu Cys Thr Glu Gly Ile Ile Glu Cys His Asn His
Ser Arg Cys Val Asn Leu Pro Gly Trp Tyr His Cys Glu Cys Arg Ser
Gly Phe His Asp Asp Gly Thr Tyr Ser Leu Ser Gly Glu Ser Cys Ile
Asp Ile Asp Glu Cys Ala Leu Arg Thr His Thr Cys Trp Asn Asp Ser
Ala Cys Ile Asn Leu Ala Gly Gly Phe Asp Cys Leu Cys Pro Ser Gly
Pro Ser Cys Ser Gly Asp Cys Pro His Glu Gly Gly Leu Lys Arg Asn
Gly Gln Val Trp Thr Leu Lys Glu Asp Arg Cys Ser Val Cys Ser Cys
Lys Asp Gly Lys Ile Phe Cys Arg Arg Thr Ala Cys Asp Cys Gln Asn
Pro Ser Val Asp Leu Phe Cys Cys Pro Glu Cys Asp Thr Arg Val Thr
Ser Gln Cys Leu Asp Gln Asn Gly His Lys Leu Tyr Arg Ser Gly Asp
Asn Trp Thr His Ser Cys Gln Gln Cys Arg Cys Leu Glu Gly Glu Val
Asp Cys Trp Pro Leu Thr Cys Pro Arg Leu Ser Cys Glu Tyr Thr Ala
Ile Leu Glu Gly Glu Cys Cys Pro Arg Cys Val Ser Asp Pro Cys Leu
Ala Asp Asn Ile Val Tyr Asp Ile Arg Glu Thr Cys Leu Asp Ser Tyr
Gly Val Ser Arg Leu Ser Gly Ser Val Trp Thr Leu Ala Gly Ser Pro
Cys Thr Thr Cys Lys Cys Lys Asn Gly Ser Val Cys Cys Ser Val Asp
Leu Glu Cys Leu His Asn Asn
Equus caballus NELL1 isoform 2 nucleotide sequence (SEQ ID NO: 7) and
translated amino acid sequence (SEP ID NO: 8)

| | |
|---|---|
| atg ggc ttt ggg atg gac ccc gac ctt caa atg gat att atc acc gag | 48 |
| Met Gly Phe Gly Met Asp Pro Asp Leu Gln Met Asp Ile Ile Thr Glu | |
| ctc gac ctc gtg aac acc acc ctt gga gtc act cag gtg tcc gga ctg | 96 |
| Leu Asp Leu Val Asn Thr Thr Leu Gly Val Thr Gln Val Ser Gly Leu | |
| cac aat gcc agc aaa gca ttt tta ttt caa gat gta gag aga gag atc | 144 |
| His Asn Ala Ser Lys Ala Phe Leu Phe Gln Asp Val Glu Arg Glu Ile | |
| cat gca gcc cca cac gtg agt gag aaa tta att cag ctg ttc cgg aat | 192 |
| His Ala Ala Pro His Val Ser Glu Lys Leu Ile Gln Leu Phe Arg Asn | |
| aag agt gaa ttc acc ttt ttg gcc act gtg cag cag aag ccg tca act | 240 |
| Lys Ser Glu Phe Thr Phe Leu Ala Thr Val Gln Gln Lys Pro Ser Thr | |
| tca gga gtg ata ctg tcc att cga gaa ctg gaa aac agt tat ttt gaa | 288 |
| Ser Gly Val Ile Leu Ser Ile Arg Glu Leu Glu Asn Ser Tyr Phe Glu | |
| ctg gag agc agt ggc ctg aga gat gag att cga tat cac tac aca cac | 336 |
| Leu Glu Ser Ser Gly Leu Arg Asp Glu Ile Arg Tyr His Tyr Thr His | |
| aag ggg aag ccc agg aca gag gca ctt ccc tac cgg atg gcg gac gga | 384 |
| Lys Gly Lys Pro Arg Thr Glu Ala Leu Pro Tyr Arg Met Ala Asp Gly | |
| cgg tgg cac aag gtg gcg ctg tca gtt agc gcc tct cat ctc ctg ctc | 432 |
| Arg Trp His Lys Val Ala Leu Ser Val Ser Ala Ser His Leu Leu Leu | |
| cac atc gac tgc aac agg att tat gaa cgt gtg ata gac act cct gag | 480 |
| His Ile Asp Cys Asn Arg Ile Tyr Glu Arg Val Ile Asp Thr Pro Glu | |
| acc aac ctc ccc cca gga agc aat ttg tgg ctg ggt cag cga aac caa | 528 |
| Thr Asn Leu Pro Pro Gly Ser Asn Leu Trp Leu Gly Gln Arg Asn Gln | |
| aag cac ggc tta ttc aaa gga atc atc caa gat gga aaa atc atc ttc | 576 |
| Lys His Gly Leu Phe Lys Gly Ile Ile Gln Asp Gly Lys Ile Ile Phe | |
| atg ccg aat gga tac ata aca cag tgt ccg aac ctg aat cgc act tgc | 624 |
| Met Pro Asn Gly Tyr Ile Thr Gln Cys Pro Asn Leu Asn Arg Thr Cys | |
| cca acg tgc agt gat ttc tta agc ctg gtg caa gga atc atg gat tta | 672 |
| Pro Thr Cys Ser Asp Phe Leu Ser Leu Val Gln Gly Ile Met Asp Leu | |
| caa gag ctt ctg gcc aag atg act gcg aaa cta aat tat gca gag aca | 720 |
| Gln Glu Leu Leu Ala Lys Met Thr Ala Lys Leu Asn Tyr Ala Glu Thr | |
| cga ctt agt caa ttg gaa aac tgc cac tgc gag aag acc tgt caa gtg | 768 |
| Arg Leu Ser Gln Leu Glu Asn Cys His Cys Glu Lys Thr Cys Gln Val | |
| agt gga ctg ctc tat aga gac cag gac tcc tgg gtt gat ggc gat cac | 816 |
| Ser Gly Leu Leu Tyr Arg Asp Gln Asp Ser Trp Val Asp Gly Asp His | |
| tgt agg aac tgc acg tgc aaa agc ggc gct gtg gaa tgt cgg agg atg | 864 |
| Cys Arg Asn Cys Thr Cys Lys Ser Gly Ala Val Glu Cys Arg Arg Met | |
| tct tgt ccc cct ctc aat tgc tcc cca gac tcc ctc cct gtg cac gtt | 912 |
| Ser Cys Pro Pro Leu Asn Cys Ser Pro Asp Ser Leu Pro Val His Val | |
| gcc ggc cag tgc tgt aag gtc tgc cga cca aaa tgt atc tac gga ggg | 960 |
| Ala Gly Gln Cys Cys Lys Val Cys Arg Pro Lys Cys Ile Tyr Gly Gly | |
| aaa gtc ctt gca gaa ggc cag cgg att tta acc aag agc tgt cgg gaa | 1008 |
| Lys Val Leu Ala Glu Gly Gln Arg Ile Leu Thr Lys Ser Cys Arg Glu | |
| tgc cga ggt gga gtt tta gtg aaa att aca gaa gcg tgc cct cct ttg | 1056 |
| Cys Arg Gly Gly Val Leu Val Lys Ile Thr Glu Ala Cys Pro Pro Leu | |
| aac tgc tca gac aag gat cac att ctc cca gag aat cag tgc tgc agc | 1104 |
| Asn Cys Ser Asp Lys Asp His Ile Leu Pro Glu Asn Gln Cys Cys Ser | |
| gtc tgc aga ggt cat aac ttt tgt gcg gaa gga cct aaa tgt ggt gaa | 1152 |
| Val Cys Arg Gly His Asn Phe Cys Ala Glu Gly Pro Lys Cys Gly Glu | |
| aat tca gag tgc aaa aac tgg aat aca aaa gct act tgc gag tgc aag | 1200 |
| Asn Ser Glu Cys Lys Asn Trp Asn Thr Lys Ala Thr Cys Glu Cys Lys | |
| aat ggt tat atc tct gtc cag ggg gac tcc gcc tac tgt gaa gat atc | 1248 |
| Asn Gly Tyr Ile Ser Val Gln Gly Asp Ser Ala Tyr Cys Glu Asp Ile | |
| gat gag tgt gct gct aag atg cat tac tgt cgt gcc aat act gtg tgt | 1296 |
| Asp Glu Cys Ala Ala Lys Met His Tyr Cys Arg Ala Asn Thr Val Cys | |
| gtc aac ctg cct ggg tta tat cgg tgt gac tgt gtc ccg gga tac att | 1344 |
| Val Asn Leu Pro Gly Leu Tyr Arg Cys Asp Cys Val Pro Gly Tyr Ile | |
| cgc gtg gat gat ttc tct tgt aca gaa cat gac gaa tgt ggc agc ggg | 1392 |
| Arg Val Asp Asp Phe Ser Cys Thr Glu His Asp Glu Cys Gly Ser Gly | |
| cag cac aac tgt gat gag aat gcc atc tgc acc aac act gtc cag gga | 1440 |
| Gln His Asn Cys Asp Glu Asn Ala Ile Cys Thr Asn Thr Val Gln Gly | |
| cac agc tgc acc tgc aaa ccg ggc tac gtg ggg aat ggg acc agc tgc | 1488 |
| His Ser Cys Thr Cys Lys Pro Gly Tyr Val Gly Asn Gly Thr Ser Cys | |
| aga gcg ttc tgc gaa gag ggc tgc aga tat ggc ggg aca tgc gtg gct | 1536 |
| Arg Ala Phe Cys Glu Glu Gly Cys Arg Tyr Gly Gly Thr Cys Val Ala | |
| cct aac aaa tgt gtc tgt cct tct gga ttc aca gga agc cac tgt gag | 1584 |
| Pro Asn Lys Cys Val Cys Pro Ser Gly Phe Thr Gly Ser His Cys Glu | |
| aaa gac att gat gaa tgt gcc tta aga act cac acc tgt tgg aat gat | 1632 |
| Lys Asp Ile Asp Glu Cys Ala Leu Arg Thr His Thr Cys Trp Asn Asp | |
| tct gcc tgc atc aac ttg gca ggg ggc ttc gac tgc ctg tgt ccc tca | 1680 |
| Ser Ala Cys Ile Asn Leu Ala Gly Gly Phe Asp Cys Leu Cys Pro Ser | |
| ggg cca tcc tgc tct ggt gac tgc ccc cac gaa gga gga ctg aag cgc | 1728 |
| Gly Pro Ser Cys Ser Gly Asp Cys Pro His Glu Gly Gly Leu Lys Arg | |
| aac ggg cag gtg tgg acc ctg aaa gaa gac agg tgt tct gtg tgt tcc | 1776 |
| Asn Gly Gln Val Trp Thr Leu Lys Glu Asp Arg Cys Ser Val Cys Ser | |
| tgc aag gat ggg aag ata ttc tgc cga cgg aca gct tgt gat tgc cag | 1824 |
| Cys Lys Asp Gly Lys Ile Phe Cys Arg Arg Thr Ala Cys Asp Cys Gln | |
| aat cca agc gtt gac ctt ttc tgt tgc cca gag tgt gac acc agg gtc | 1872 |
| Asn Pro Ser Val Asp Leu Phe Cys Cys Pro Glu Cys Asp Thr Arg Val | |
| aca agt caa tgt tta gac caa aat gga cac aag ctc tat cga agt gga | 1920 |
| Thr Ser Gln Cys Leu Asp Gln Asn Gly His Lys Leu Tyr Arg Ser Gly | |
| gac aat tgg act cac agc tgt cag cag tgc cgg tgt ctg gaa gga gag | 1968 |
| Asp Asn Trp Thr His Ser Cys Gln Gln Cys Arg Cys Leu Glu Gly Glu | |
| gta gat tgc tgg cca ctc act tgc ccc aga ttg agc tgt gag tac aca | 2016 |
| Val Asp Cys Trp Pro Leu Thr Cys Pro Arg Leu Ser Cys Glu Tyr Thr | |
| gcc atc ttg gaa ggg gag tgt tgt cca cgc tgt gtc agc gac ccc tgc | 2064 |
| Ala Ile Leu Glu Gly Glu Cys Cys Pro Arg Cys Val Ser Asp Pro Cys | |
| ctg gcg gat aac atc gtc tat gac atc aga gaa act tgc ctg gac agc | 2112 |
| Leu Ala Asp Asn Ile Val Tyr Asp Ile Arg Glu Thr Cys Leu Asp Ser | |
| tat gga gtt tca agg ctt agt ggc tca gtg tgg aca ttg gct gga tct | 2160 |
| Tyr Gly Val Ser Arg Leu Ser Gly Ser Val Trp Thr Leu Ala Gly Ser | |
| ccc tgc acg acc tgc aaa tgc aag aat gga agt gtc tgc tgt tct gtg | 2208 |
| Pro Cys Thr Thr Cys Lys Cys Lys Asn Gly Ser Val Cys Cys Ser Val | |
| gat ttg gag tgt ctt cat aat aat tga aggatttaaa atggactcat | 2255 |
| Asp Leu Glu Cys Leu His Asn Asn * | |
| gatcgccaga gaaaaatgga caaatgacca | 2285 |

Equus caballus NELL1 isoform 2 amino acid sequence (SEQ ID NO: 8)
Met Gly Phe Gly Met Asp Pro Asp Leu gln Met Asp Ile Ile Thr Glu
Leu Asp Leu Val Asn Thr Thr Leu Gly Val Thr Gln Val Ser Gly Leu
His Asn Ala Ser Lys Ala Phe Leu Phe Gln Asp Val Glu Arg Glu Ile
His Ala Ala Pro His Val Ser Glu Lys Leu Ile Gln Leu Phe Arg Asn
Lys Ser Glu Phe Thr Phe Leu Ala Thr Val Gln Gln Lys Pro Ser Thr
Ser Gly Val Ile Leu Ser Ile Arg Glu Leu Glu Asn Ser Tyr Phe Glu
Leu Glu Ser Ser Gly Leu Arg Asp Glu Ile Arg Tyr His Tyr Thr His
Lys Gly Lys Pro Arg Thr Glu Ala Leu Pro Tyr Arg Met Ala Asp Gly
Arg Trp His Lys Val Ala Leu Ser Val Ser Ala Ser His Leu Leu Leu
His Ile Asp Cys Asn Arg Ile Tyr Glu Arg Val Ile Asp Thr Pro Glu
Thr Asn Leu Pro Pro Gly Ser Asn Leu Trp Leu Gly Gln Arg Asn Gln
Lys His Gly Leu Phe Lys Gly Ile Ile Gln Asp Gly Lys Ile Ile Phe
Met Pro Asn Gly Tyr Ile Thr Gln Cys Pro Asn Leu Asn Arg Thr Cys
Pro Thr Cys Ser Asp Phe Leu Ser Leu Val Gln Gly Ile Met Asp Leu
Gln Glu Leu Leu Ala Lys Met Thr Ala Lys Leu Asn Tyr Ala Glu Thr
Arg Leu Ser Gln Leu Glu Asn Cys His Cys Glu Lys Thr Cys Gln Val
Ser Gly Leu Leu Tyr Arg Asp Gln Asp Ser Trp Val Asp Gly Asp His
Cys Arg Asn Cys Thr Cys Lys Ser Gly Ala Val Glu Cys Arg Arg Met
Ser Cys Pro Pro Leu Asn Cys Ser Pro Asp Ser Leu Pro Val His Val
Ala Gly Gln Cys Cys Lys Val Cys Arg Pro Lys Cys Ile Tyr Gly Gly
Lys Val Leu Ala Glu Gly Gln Arg Ile Leu Thr Lys Ser Cys Arg Glu
Cys Arg Gly Gly Val Leu Val Lys Ile Thr Glu Ala Cys Pro Pro Leu
Asn Cys Ser Asp Lys Asp His Ile Leu Pro Glu Asn Gln Cys Cys Ser
Val Cys Arg Gly His Asn Phe Cys Ala Glu Gly Pro Lys Cys Gly Glu
Asn Ser Glu Cys Lys Asn Trp Asn Thr Lys Ala Thr Cys Glu Cys Lys
Asn Gly Tyr Ile Ser Val Gln Gly Asp Ser Ala Tyr Cys Glu Asp Ile
Asp Glu Cys Ala Ala Lys Met His Tyr Cys Arg Ala Asn Thr Val Cys
Val Asn Leu Pro Gly Leu Tyr Arg Cys Asp Cys Val Pro Gly Tyr Ile
Arg Val Asp Asp Phe Ser Cys Thr Glu His Asp Glu Cys Gly Ser Gly
Gln His Asn Cys Asp Glu Asn Ala Ile cys Thr Asn Thr Val Gln Gly
His Ser Cys Thr Cys Lys Pro Gly Tyr Val Gly Asn Gly Thr Ser Cys
Arg Ala Phe Cys Glu Glu Gly Cys Arg Tyr Gly Gly Thr Cys Val Ala
Pro Asn Lys Cys Val Cys Pro Ser Gly Phe Thr Gly Ser His Cys Glu
Lys Asp Ile Asp Glu Cys Ala Leu Arg Thr His Thr Cys Trp Asn Asp
Ser Ala Cys Ile Asn Leu Ala Gly Gly Phe Asp Cys Leu Cys Pro Ser
Gly Pro Ser Cys Ser Gly Asp Cys Pro His Glu Gly Gly Leu Lys Arg
Asn Gly Gln Val Trp Thr Leu Lys Glu Asp Arg Cys Ser Val Cys Ser
Cys Lys Asp Gly Lys Ile Phe Cys Arg Arg Thr Ala Cys Asp Cys Gln
Asn Pro Ser Val Asp Leu Phe Cys Cys Pro Glu Cys Asp Thr Arg Val
Thr Ser Gln Cys Leu Asp Gln Asn Gly His Lys Leu Tyr Arg Ser Gly
Asp Asn Trp Thr His Ser Cys Gln Gln Cys Arg Cys Leu Glu Gly Glu
Val Asp Cys Trp Pro Leu Thr Cys Pro Arg Leu Ser Cys Glu Tyr Thr
Ala Ile Leu Glu Gly Glu Cys Cys Pro Arg Cys Val Ser Asp Pro Cys
Leu Ala Asp Asn Ile Val Tyr Asp Ile Arg Glu Thr Cys Leu Asp Ser
Tyr Gly Val Ser Arg Leu Ser Gly Ser Val Trp Thr Leu Ala Gly Ser
Pro Cys Thr Thr Cys Lys Cys Lys Asn Gly Ser Val Cys Cys Ser Val
Asp Leu Glu Cys Leu His Asn Asn
Mus musculus NELL1 nucleotide sequence (SEQ ID NO: 9) and translated;
ID NO: 10)

| gcgttggtgc gccctgcttg gcggggggcc tccggagcg atg ccg atg gat gtg | 54 | |
|---|---|---|
| Met Pro Met Asp Val | | |
| att tta gtt ttg tgg ttc tgt gtg tgc acc gcc agg aca gtg ctg ggc | 02 | |
| Ile Leu Val Leu Trp Phe Cys Val Cys Thr Ala Arg Thr Val Leu Gly | | |
| ttt ggg atg gac cct gac ctt cag atg gac atc atc act gaa ctt gac | 150 | |
| Phe Gly Met Asp Pro Asp Leu Gln Met Asp Ile Ile Thr Glu Leu Asp | | |
| ctt gtg aac acc acc ctg ggc gtc act cag gtg gct gga cta cac aat | 198 | |
| Leu Val Asn Thr Thr Leu Gly Val Thr Gln Val Ala Gly Leu His Asn | | |
| gcc agt aag gca ttt ctg ttt caa gat gta cag aga gag atc cac tca | 246 | |
| Ala Ser Lys Ala Phe Leu Phe Gln Asp Val Gln Arg Glu Ile His Ser | | |
| gcc cct cat gtg agt gag aag ctg atc cag cta ttc cgg aat aag agt | 294 | |
| Ala Pro His Val Ser Glu Lys Leu Ile Gln Leu Phe Arg Asn Lys Ser | | |
| gag ttt acc ttt ttg gct aca gtg cag cag aag ccg tcc acc tca ggg | 342 | |
| Glu Phe Thr Phe Leu Ala Thr Val Gln Gln Lys Pro Ser Thr Ser Gly | | |
| gtg ata ctg tcg atc cgg gag ctg gaa cac agc tat ttt gaa ctg gag | 390 | |
| Val Ile Leu Ser Ile Arg Glu Leu Glu His Ser Tyr Phe Glu Leu Glu | | |
| agc agt ggc cca aga gaa gag ata cgc tat cat tac atc cat ggc ggc | 438 | |
| Ser Ser Gly Pro Arg Glu Glu Ile Arg Tyr His Tyr Ile His Gly Gly | | |
| aag ccc agg act gag gcc ctt ccc tac cgc atg gcc gat gga cag tgg | 486 | |
| Lys Pro Arg Thr Glu Ala Leu Pro Tyr Arg Met Ala Asp Gly Gln Trp | | |
| cac aag gtc gcg ctg tct gtg agc gcc tct cac ctc cta ctc cat gtc | 534 | |
| His Lys Val Ala Leu Ser Val Ser Ala Ser His Leu Leu Leu His Val | | |
| gac tgc aat agg att tat gag cgt gtg ata gat cct ccg gag acc aac | 582 | |
| Asp Cys Asn Arg Ile Tyr Glu Arg Val Ile Asp Pro Pro Glu Thr Asn | | |
| ctt cct cca gga agc aat cta tgg ctt ggg caa cgt aat caa aag cat | 630 | |
| Leu Pro Pro Gly Ser Asn Leu Trp Leu Gly Gln Arg Asn Gln Lys His | | |
| ggc ttt ttc aaa gga atc atc caa gat ggc aag atc atc ttc atg ccg | 678 | |
| Gly Phe Phe Lys Gly Ile Ile Gln Asp Gly Lys Ile Ile Phe Met Pro | | |
| aac ggc ttc atc aca cag tgc ccc aac cta aat cgc act tgc cca aca | 726 | |
| Asn Gly Phe Ile Thr Gln Cys Pro Asn Leu Asn Arg Thr Cys Pro Thr | | |
| tgc agt gat ttc ctg agc ctg gtt caa gga ata atg gat ttg caa gag | 774 | |
| Cys Ser Asp Phe Leu Ser Leu Val Gln Gly Ile Met Asp Leu Gln Glu | | |
| ctt ttg gcc aag atg act gca aaa ctg aat tat gca gag acg aga ctt | 822 | |
| Leu Leu Ala Lys Met Thr Ala Lys Leu Asn Tyr Ala Glu Thr Arg Leu | | |
| ggt caa ctg gaa aat tgc cac tgt gag aag acc tgc caa gtg agt ggg | 870 | |
| Gly Gln Leu Glu Asn Cys His Cys Glu Lys Thr Cys Gln Val Ser Gly | | |
| ctg ctc tac agg gac caa gac tcc tgg gta gat ggt gac aac tgc agg | 918 | |
| Leu Leu Tyr Arg Asp Gln Asp Ser Trp Val Asp Gly Asp Asn Cys Arg | | |
| aac tgc aca tgc aaa agt ggt gct gtg gag tgc cga agg atg tcc tgt | 966 | |
| Asn Cys Thr Cys Lys Ser Gly Ala Val Glu Cys Arg Arg Met Ser Cys | | |
| ccc cca ctc aac tgt tcc cca gac tca ctt cct gtg cat att tct ggc | 1014 | |
| Pro Pro Leu Asn Cys Ser Pro Asp Ser Leu Pro Val His Ile Ser Gly | | |
| caa tgt tgt aaa gtt tgc aga cca aaa tgt atc tat gga gga aaa gtt | 1062 | |
| Gln Cys Cys Lys Val Cys Arg Pro Lys Cys Ile Tyr Gly Gly Lys Val | | |
| ctt gct gag ggc cag cgg att tta acc aag acc tgc cgg gaa tgt cga | 1110 | |
| Leu Ala Glu Gly Gln Arg Ile Leu Thr Lys Thr Cys Arg Glu Cys Arg | | |
| ggt gga gtc ttg gta aaa atc aca gaa gct tgc cct cct ttg aac tgc | 1158 | |
| Gly Gly Val Leu Val Lys Ile Thr Glu Ala Cys Pro Pro Leu Asn Cys | | |
| tca gag aag gat cat att ctt ccg gag aac cag tgc tgc agg gtc tgc | 1206 | |
| Ser Glu Lys Asp His Ile Leu Pro Glu Asn Gln Cys Cys Arg Val Cys | | |
| cga ggt cat aac ttc tgt gca gaa gca cct aag tgt gga gaa aac tcg | 1254 | |
| Arg Gly His Asn Phe Cys Ala Glu Ala Pro Lys Cys Gly Glu Asn Ser | | |
| gaa tgc aaa aat tgg aat aca aaa gcg act tgt gag tgc aag aat gga | 1302 | |
| Glu Cys Lys Asn Trp Asn Thr Lys Ala Thr Cys Glu Cys Lys Asn Gly | | |
| tac atc tct gtc cag ggc aac tct gca tac tgt gaa gat atc gat gag | 1350 | |
| Tyr Ile Ser Val Gln Gly Asn Ser Ala Tyr Cys Glu Asp Ile Asp Glu | | |
| tgt gca gca aag atg cac tac tgt cat gcc aac acg gtg tgt gtc aac | 1398 | |
| Cys Ala Ala Lys Met His Tyr Cys His Ala Asn Thr Val Cys Val Asn | | |
| ttg ccg ggg tta tat cgc tgt gac tgc atc cca gga tac atc cgt gtg | 1446 | |
| Leu Pro Gly Leu Tyr Arg Cys Asp Cys Ile Pro Gly Tyr Ile Arg Val | | |
| gat gac ttc tct tgt acg gag cat gat gat tgt ggc agc gga caa cac | 1494 | |
| Asp Asp Phe Ser Cys Thr Glu His Asp Asp Cys Gly Ser Gly Gln His | | |
| aac tgt gac aaa aat gcc atc tgt acc aac aca gtc cag gga cac agc | 1542 | |
| Asn Cys Asp Lys Asn Ala Ile Cys Thr Asn Thr Val Gln Gly His Ser | | |
| tgt acc tgc cag cca ggc tac gtg gga aat ggt act gtc tgc aaa gca | 1590 | |
| Cys Thr Cys Gln Pro Gly Tyr Val Gly Asn Gly Thr Val Cys Lys Ala | | |
| ttc tgt gaa gag ggt tgc aga tac gga ggt acc tgt gtg gcc cct aac | 1638 | |
| Phe Cys Glu Glu Gly Cys Arg Tyr Gly Gly Thr Cys Val Ala Pro Asn | | |
| aaa tgt gtc tgt cct tct gga ttc aca gga agc cac tgt gag aaa gat | 1686 | |
| Lys Cys Val Cys Pro Ser Gly Phe Thr Gly Ser His Cys Glu Lys Asp | | |
| att gat gaa tgt gca gag gga ttc gtt gag tgc cac aac cac tcc cgc | 1734 | |
| Ile Asp Glu Cys Ala Glu Gly Phe Val Glu Cys His Asn His Ser Arg | | |
| tgc gtt aac ctt cca ggg tgg tac cac tgt gag tgc aga agc ggt ttc | 1782 | |
| Cys Val Asn Leu Pro Gly Trp Tyr His Cys Glu Cys Arg Ser Gly Phe | | |
| cat gac gat ggg acc tat tca ctg tcc ggg gag tcc tgc att gat att | 1830 | |
| His Asp Asp Gly Thr Tyr Ser Leu Ser Gly Glu Ser Cys Ile Asp Ile | | |
| gat gaa tgt gcc tta aga act cac act tgt tgg aat gac tct gcc tgc | 1878 | |
| Asp Glu Cys Ala Leu Arg Thr His Thr Cys Trp Asn Asp Ser Ala Cys | | |
| atc aac tta gca gga gga ttt gac tgc ctg tgt ccc tct ggg ccc tcc | 1926 | |
| Ile Asn Leu Ala Gly Gly Phe Asp Cys Leu Cys Pro Ser Gly Pro Ser | | |
| tgc tct ggt gac tgt ccc cac gaa ggg ggg ctg aag cat aat ggg cag | 1974 | |
| Cys Ser Gly Asp Cys Pro His Glu Gly Gly Leu Lys His Asn Gly Gln | | |
| gtg tgg att ctg aga gaa gac agg tgt tca gtc tgt tcc tgt aag gat | 2022 | |
| Val Trp Ile Leu Arg Glu Asp Arg Cys Ser Val Cys Ser Cys Lys Asp | | |
| ggg aag ata ttc tgc cgg cgg aca gct tgt gat tgc cag aat cca aat | 2070 | |
| Gly Lys Ile Phe Cys Arg Arg Thr Ala Cys Asp Cys Gln Asn Pro Asn | | |
| gtt gac ctt ttc tgc tgc cca gag tgt gac acc agg gtc act agc caa | 2118 | |
| Val Asp Leu Phe Cys Cys Pro Glu Cys Asp Thr Arg Val Thr Ser Gln | | |
| tgt tta gat caa agc gga cag aag ctc tat cga agt gga gac aac tgg | 2166 | |
| Cys Leu Asp Gln Ser Gly Gln Lys Leu Tyr Arg Ser Gly Asp Asn Trp | | |
| acc cac agc tgc cag cag tgc cga tgt ctg gaa gga gag gca gac tgc | 2214 | |
| Thr His Ser Cys Gln Gln Cys Arg Cys Leu Glu Gly Glu Ala Asp Cys | | |
| tgg cct cta gct tgc cct agt ttg agc tgt gaa tac aca gcc atc ttt | 2262 | |
| Trp Pro Leu Ala Cys Pro Ser Leu Ser Cys Glu Tyr Thr Ala Ile Phe | | |
| gaa gga gag tgt tgt ccc cgc tgt gtc agt gac ccc tgc ctg gct gat | 2310 | |
| Glu Gly Glu Cys Cys Pro Arg Cys Val Ser Asp Pro Cys Leu Ala Asp | | |
| aat att gcc tat gac atc aga aaa act tgc ctg gac agc tct ggt att | 2358 | |
| Asn Ile Ala Tyr Asp Ile Arg Lys Thr Cys Leu Asp Ser Ser Gly Ile | | |
| tcg agg ctg agc ggc gca gtg tgg aca atg gct gga tct ccc tgt aca | 2406 | |
| Ser Arg Leu Ser Gly Ala Val Trp Thr Met Ala Gly Ser Pro Cys Thr | | |
| acc tgt caa tgc aag aat ggg aga gtc tgc tgc tct gtg gat ctg gtg | 2454 | |
| Thr Cys Gln Cys Lys Asn Gly Arg Val Cys Cys Ser Val Asp Leu Val | | |
| tgt ctt gag aat aac tga agattttaaa tggactcatc acatgagaaa | 2502 | |
| Cys Leu Glu Asn Asn * | | |
| atggacaaaa tgaccatcca acctgaggaa gaggaggggc tgatttcttt ttctttttaa | 2562 | |
| ccacagtcaa ttaccaaagt ctccatcaga ggaaggcgtt tgggttgcct ttaccacttt | 2622 | |
| gctcatcctt gctgacctag tctagatgcc tgcagtaccg tgtatttcgg tcgatggttg | 2682 | |
| ttgagtctcc gtgctgtaaa tcacatttcc cttgtcagat catttacaga tacatttaaa | 2742 | |
| ggattccatg ataaatgtta aagtaccttt tgtttatttt gtgtaccaac ataatagaga | 2802 | |
| cttggcacca | | 2812 |

Mus musculus NELL1 amino acid sequence (SEQ ID NO: 10)
Met Pro Met Asp Val Ile Leu Val Leu Trp Phe Cys Val Cys Thr Ala
Arg Thr Val Leu Gly Phe Gly Met Asp Pro Asp Leu Gln Met Asp Ile
Ile Thr Glu Leu Asp Leu Val Asn Thr Thr Leu Gly Val Thr Gln Val
Ala Gly Leu His Asn Ala Ser Lys Ala Phe Leu Phe Gln Asp Val Gln
Arg Glu Ile His Ser Ala Pro His Val Ser Glu Lys Leu Ile Gln Leu
Phe Arg Asn Lys Ser Glu Phe Thr Phe Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Pro Arg Glu Glu Ile Arg Tyr His
Tyr Ile His Gly Gly Lys Pro Arg Thr Glu Ala Leu Pro Tyr Arg Met
Ala Asp Gly Gln Trp His Lys Val Ala Leu Ser Val Ser Ala Ser His
Leu Leu Leu His Val Asp Cys Asn Arg Ile Tyr Glu Arg Val Ile Asp
Pro Pro Glu Thr Asn Leu Pro Pro Gly Ser Asn Leu Trp Leu Gly Gln
Arg Asn Gln Lys His Gly Phe Phe Lys Gly Ile Ile Gln Asp Gly Lys
Ile Ile Phe Met Pro Asn Gly Phe Ile Thr Gln Cys Pro Asn Leu Asn
Arg Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Gly Gln Leu Glu Asn Cys His Cys Glu Lys Thr
Cys Gln Val Ser Gly Leu Leu Tyr Arg Asp Gln Asp Ser Trp Val Asp
Gly Asp Asn Cys Arg Asn Cys Thr Cys Lys Ser Gly Ala Val Glu Cys
Arg Arg Met Ser Cys Pro Pro Leu Asn Cys Ser Pro Asp Ser Leu Fro
Val His Ile Ser Gly Gln Cys Cys Lys Val Cys Arg Pro Lys Cys Ile
Tyr Gly Gly Lys Val Leu Ala Glu Gly Gln Arg Ile Leu Thr Lys Thr
Cys Arg Glu Cys Arg Gly Gly Val Leu Val Lys Ile Thr Glu Ala Cys
Pro Pro Leu Asn Cys Ser Glu Lys Asp His Ile Leu Pro Glu Asn Gln
Cys Cys Arg Val Cys Arg Gly His Asn Phe Cys Ala Glu Ala Pro Lys
Cys Gly Glu Asn Ser Glu Cys Lys Asn Trp Asn Thr Lys Ala Thr Cys
Glu Cys Lys Asn Gly Tyr Ile Ser Val Gln Gly Asn Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Ile Pro
Gly Tyr Ile Arg Val Asp Asp Phe Ser Cys Thr Glu His Asp Asp Cys
Gly Ser Gly Gln His Asn Cys Asp Lys Asn Ala Ile Cys Thr Asn Thr
Val Gln Gly His Ser Cys Thr Cys Gln Pro Gly Tyr Val Gly Asn Gly
Thr Val Cys Lys Ala Phe Cys Glu Glu Gly Cys Arg Tyr Gly Gly Thr
Cys Val Ala Pro Asn Lys Cys Val Cys Pro Ser Gly Phe Thr Gly Ser
His Cys Glu Lys Asp Ile Asp Glu Cys Ala Glu Gly Phe Val Glu Cys
His Asn His Ser Arg Cys Val Asn Leu Pro Gly Trp Tyr His Cys Glu
Cys Arg Ser Gly Phe His Asp Asp Gly Thr Tyr Ser Leu Ser Gly Glu
Ser Cys Ile Asp Ile Asp Glu Cys Ala Leu Arg Thr His Thr Cys Trp
Asn Asp Ser Ala Cys Ile Asn Leu Ala Gly Gly Phe Asp Cys Leu Cys
Pro Ser Gly Pro Ser Cys Ser Gly Asp Cys Pro His Glu Gly Gly Leu
Lys His Asn Gly Gln Val Trp Ile Leu Arg Glu Asp Arg Cys Ser Val
Cys Ser Cys Lys Asp Gly Lys Ile Phe Cys Arg Arg Thr Ala Cys Asp
Cys Gln Asn Pro Asn Val Asp Leu Phe Cys Cys Pro Glu Cys Asp Thr
Arg Val Thr Ser Gln Cys Leu Asp Gln Ser Gly Gln Lys Leu Tyr Arg
Ser Gly Asp Asn Trp Thr His Ser Cys Gln Gln Cys Arg Cys Leu Glu
Gly Glu Ala Asp Cys Trp Pro Leu Ala Cys Pro Ser Leu Ser Cys Glu
Tyr Thr Ala Ile Phe Glu Gly Glu Cys Cys Pro Arg Cys Val Ser Asp
Pro Cys Leu Ala Asp Asn Ile Ala Tyr Asp Ile Arg Lys Thr Cys Leu
Asp Ser Ser Gly Ile Ser Arg Leu Ser Gly Ala Val Trp Thr Met Ala
Gly Ser Pro Cys Thr Thr Cys Gln Cys Lys Asn Gly Arg Val Cys Cys
Ser Val Asp Leu Val Cys Leu Glu Asn Asn
Rattus norvesicus NELL1 nucleotide sequence (SEQ ID NO: 11) and translated
amino acid sequence (SEQ ID NO: 12)

| | |
|---|---|
| aagcactggt ttcttgttag cgttggtgcg ccctgcttgg cgggggttct ccggagcg | 58 |
| atg ccg atg gat gtg att tta gtt ttg tgg ttc tgt gta tgc acc gcc | 106 |
| Met Pro Met Asp Val Ile Leu Val Leu Trp Phe Cys Val Cys Thr Ala | |
| agg aca gtg ttg ggc ttt ggg atg gac cct gac ctt cag ctg gac atc | 154 |
| Arg Thr Val Leu Gly Phe Gly Met Asp Pro Asp Leu Gln Leu Asp Ile | |
| atc tca gag ctc gac ctg gtg aac acc acc ctg gga gtc acg cag gtg | 202 |
| Ile Ser Glu Leu Asp Leu Val Asn Thr Thr Leu Gly Val Thr Gln Val | |
| gct gga ctg cac aac gcc agt aaa gca ttt cta ttt caa gat gta cag | 250 |
| Ala Gly Leu His Asn Ala Ser Lys Ala Phe Leu Phe Gln Asp Val Gln | |
| aga gag atc cat tcg gcc cct cac gtg agt gag aag ctg atc cag cta | 298 |
| Arg Glu Ile His Ser Ala Pro His Val Ser Glu Lys Leu Ile Gln Leu | |
| ttc cgg aat aag agc gag ttc acc ttt ttg gct aca gtg cag cag aaa | 346 |
| Phe Arg Asn Lys Ser Glu Phe Thr Phe Leu Ala Thr Val Gln Gln Lys | |
| cca tcc acc tca ggg gtg ata ctg tcc atc cgg gag ctg gag cac agc | 394 |
| Pro Ser Thr Ser Gly Val Ile Leu Ser Ile Arg Glu Leu Glu His Ser | |
| tat ttt gaa ctg gag agc agt ggc cca aga gaa gag ata cgc tac cat | 442 |
| Tyr Phe Glu Leu Glu Ser Ser Gly Pro Arg Glu Glu Ile Arg Tyr His | |
| tac ata cat ggt gga aag ccc agg act gag gcc ctt ccc tac cgc atg | 490 |
| Tyr Ile His Gly Gly Lys Pro Arg Thr Glu Ala Leu Pro Tyr Arg Met | |
| gca gac gga caa tgg cac aag gtc gcg ctg tca gtg agc gcc tct cac | 538 |
| Ala Asp Gly Gln Trp His Lys Val Ala Leu Ser Val Ser Ala Ser His | |
| ctc ctg ctc cac atc gac tgc aat agg att tac gag cgt gtg ata gac | 586 |
| Leu Leu Leu His Ile Asp Cys Asn Arg Ile Tyr Glu Arg Val Ile Asp | |
| cct ccg gag acc aac ctt cct cca gga agc aat ctg tgg ctt ggg caa | 634 |
| Pro Pro Glu Thr Asn Leu Pro Pro Gly Ser Asn Leu Trp Leu Gly Gln | |
| cgt aac caa aag cat ggc ttt ttc aaa gga atc atc caa gat ggt aag | 682 |
| Arg Asn Gln Lys His Gly Phe Phe Lys Gly Ile Ile Gln Asp Gly Lys | |
| atc atc ttc atg ccg aat ggt ttc atc aca cag tgt ccc aac ctc aat | 730 |
| Ile Ile Phe Met Pro Asn Gly Phe Ile Thr Gln Cys Pro Asn Leu Asn | |
| cgc act tgc cca aca tgc agt gac ttc ctg agc ctg gtt caa gga ata | 778 |
| Arg Thr Cys Pro Thr Cys Ser Asp Phe Leu Ser Leu Val Gln Gly Ile | |
| atg gat ttg caa gag ctt ttg gcc aag atg act gca aaa ctg aat tat | 826 |
| Met Asp Leu Gln Glu Leu Leu Ala Lys Met Thr Ala Lys Leu Asn Tyr | |
| gca gag acg aga ctt ggt caa ctg gaa aat tgc cac tgt gag aag acc | 874 |
| Ala Glu Thr Arg Leu Gly Gln Leu Glu Asn Cys His Cys Glu Lys Thr | |
| tgc caa gtg agt ggg ctg ctc tac agg gac caa gac tcc tgg gtg gat | 922 |
| Cys Gln Val Ser Gly Leu Leu Tyr Arg Asp Gln Asp Ser Trp Val Asp | |
| ggt gac aac tgt ggg aac tgc acg tgc aaa agt ggt gcc gtg gag tgc | 970 |
| Gly Asp Asn Cys Gly Asn Cys Thr Cys Lys Ser Gly Ala Val Glu Cys | |
| cgc agg atg tcc tgt ccc ccg ctc aac tgt tcc ccg gac tca ctt cct | 1018 |
| Arg Arg Met Ser Cys Pro Pro Leu Asn Cys Ser Pro Asp Ser Leu Pro | |
| gtg cac att tcc ggc cag tgt tgt aaa gtt tgc aga cca aaa tgt atc | 1066 |
| Val His Ile Ser Gly Gln Cys Cys Lys Val Cys Arg Pro Lys Cys Ile | |
| tat gga gga aaa gtt ctt gct gag ggc cag cgg att tta acc aag acc | 1114 |
| Tyr Gly Gly Lys Val Leu Ala Glu Gly Gln Arg Ile Leu Thr Lys Thr | |
| tgc cgg gaa tgt cga ggt gga gtc ttg gta aaa atc aca gaa gct tgc | 1162 |
| Cys Arg Glu Cys Arg Gly Gly Val Leu Val Lys Ile Thr Glu Ala Cys | |
| cct cct ttg aac tgc tca gca aag gat cat att ctt cca gag aat cag | 1210 |
| Pro Pro Leu Asn Cys Ser Ala Lys Asp His Ile Leu Pro Glu Asn Gln | |
| tgc tgc agg gtc tgc cca ggt cat aac ttc tgt gca gaa gca cct aag | 1258 |
| Cys Cys Arg Val Cys Pro Gly His Asn Phe Cys Ala Glu Ala Pro Lys | |
| tgc gga gaa aac tcg gaa tgc aaa aat tgg aat aca aaa gca acc tgt | 1306 |
| Cys Gly Glu Asn Ser Glu Cys Lys Asn Trp Asn Thr Lys Ala Thr Cys | |
| gag tgc aag aat gga tac atc tct gtc cag ggc aac tct gca tac tgt | 1354 |
| Glu Cys Lys Asn Gly Tyr Ile Ser Val Gln Gly Asn Ser Ala Tyr Cys | |
| gaa gat att gat gag tgt gca gct aaa atg cac tat tgt cat gcc aac | 1402 |
| Glu Asp Ile Asp Glu Cys Ala Ala Lys Met His Tyr Cys His Ala Asn | |
| acc gtg tgt gtc aac ttg ccg ggg ttg tat cgc tgt gac tgc gtc cca | 1450 |
| Thr Val Cys Val Asn Leu Pro Gly Leu Tyr Arg Cys Asp Cys Val Pro | |
| ggg tac atc cgt gtg gat gac ttc tct tgt acg gag cat gat gat tgt | 1498 |
| Gly Tyr Ile Arg Val Asp Asp Phe Ser Cys Thr Glu His Asp Asp Cys | |
| ggc agc gga caa cac aac tgc gac aaa aat gcc atc tgt acc aac aca | 1546 |
| Gly Ser Gly Gln His Asn Cys Asp Lys Asn Ala Ile Cys Thr Asn Thr | |
| gtc cag gga cac agc tgc acc tgc cag ccg ggt tac gtg gga aat ggc | 1594 |
| Val Gln Gly His Ser Cys Thr Cys Gln Pro Gly Tyr Val Gly Asn Gly | |
| acc atc tgc aaa gca ttc tgt gaa gag ggt tgc aga tac gga ggt acc | 1642 |
| Thr Ile Cys Lys Ala Phe Cys Glu Glu Gly Cys Arg Tyr Gly Gly Thr | |
| tgt gtg gct cct aac aag tgt gtc tgt cct tct gga ttc acg gga agc | 1690 |
| Cys Val Ala Pro Asn Lys Cys Val Cys Pro Ser Gly Phe Thr Gly Ser | |
| cac tgt gag aaa gat att gat gaa tgc gca gag gga ttc gtt gaa tgc | 1738 |
| His Cys Glu Lys Asp Ile Asp Glu Cys Ala Glu Gly Phe Val Glu Cys | |
| cac aac tac tcc cgc tgt gtt aac ctg cca ggg tgg tac cac tgt gag | 1786 |
| His Asn Tyr Ser Arg Cys Val Asn Leu Pro Gly Trp Tyr His Cys Glu | |
| tgc aga agc ggt ttc cat gac gat ggg acc tac tca ctg tcc ggg gag | 1834 |
| Cys Arg Ser Gly Phe His Asp Asp Gly Thr Tyr Ser Leu Ser Gly Glu | |
| tcc tgc att gat atc gat gaa tgt gcc tta aga act cac act tgt tgg | 1882 |
| Ser Cys Ile Asp Ile Asp Glu Cys Ala Leu Arg Thr His Thr Cys Trp | |
| aat gac tct gcc tgc atc aac tta gca gga gga ttt gac tgc ctg tgt | 1930 |
| Asn Asp Ser Ala Cys Ile Asn Leu Ala Gly Gly Phe Asp Cys Leu Cys | |
| ccc tct ggg ccc tcc tgc tct ggt gac tgt ccc cac gaa gga ggg ctg | 1978 |
| Pro Ser Gly Pro Ser Cys Ser Gly Asp Cys Pro His Glu Gly Gly Leu | |
| aag cat aat ggg cag gtg tgg att ctg aga gaa gac agg tgt tca gtc | 2026 |
| Lys His Asn Gly Gln Val Trp Ile Leu Arg Glu Asp Arg Cys Ser Val | |
| tgt tcc tgc aag gat ggg aag ata ttc tgc cgg cgg aca gct tgt gat | 2074 |
| Cys Ser Cys Lys Asp Gly Lys Ile Phe Cys Arg Arg Thr Ala Cys Asp | |
| tgc cag aat cca aat gtt gac ctt ttt tgc tgc cca gag tgc gat acc | 2122 |
| Cys Gln Asn Pro Asn Val Asp Leu Phe Cys Cys Pro Glu Cys Asp Thr | |
| agg gtc acc agc caa tgt tta gat caa agt gga cag aag ctc tat cga | 2170 |
| Arg Val Thr Ser Gln Cys Leu Asp Gln Ser Gly Gln Lys Leu Tyr Arg | |
| agt gga gac aac tgg acc cac agc tgc cag cag tgc cga tgt ctg gaa | 2218 |
| Ser Gly Asp Asn Trp Thr His Ser Cys Gln Gln Cys Arg Cys Leu Glu | |
| gga gag gca gac tgc tgg cct ctg gct tgc cct agt ttg ggc tgt gaa | 2266 |
| Gly Glu Ala Asp Cys Trp Pro Leu Ala Cys Pro Ser Leu Gly Cys Glu | |
| tac aca gcc atg ttt gaa ggg gag tgt tgt ccc cga tgt gtc agt gac | 2314 |
| Tyr Thr Ala Met Phe Glu Gly Glu Cys Cys Pro Arg Cys Val Ser Asp | |
| ccc tgc ctg gct ggt aat att gcc tat gac atc aga aaa act tgc ctg | 2362 |
| Pro Cys Leu Ala Gly Asn Ile Ala Tyr Asp Ile Arg Lys Thr Cys Leu | |
| gac agc ttt ggt gtt tcg agg ctg agc gga gcc gtg tgg aca atg gct | 2410 |
| Asp Ser Phe Gly Val Ser Arg Leu Ser Gly Ala Val Trp Thr Met Ala | |
| gga tct cct tgt aca acc tgc aaa tgc aag aat ggg aga gtc tgc tgc | 2458 |
| Gly Ser Pro Cys Thr Thr Cys Lys Cys Lys Asn Gly Arg Val Cys Cys | |
| tct gtg gat ctg gag tgt att gag aat aac tga agattttaaa tggactcgtc | 2511 |
| Ser Val Asp Leu Glu Cys Ile Glu Asn Asn * | |
| acgtgagaaa atgggcaaaa tgatcatccc acctgaggaa gaagaggggc tgatttcttt | 2571 |
| ttctttttaa ccacagtcaa ttaccaaagt ctccatctga ggaaggcgtt tggattgcct | 2631 |
| ttgccacttt gctcatcctt gctgacctag tctagatgcc tgcagtaccg tgcatttcgg | 2691 |
| tcgatggttg ttgagtctca gtgttgtaaa tcgcatttcc ctcgtcagat catttacaga | 2751 |
| tacatttaaa ggggttccat gataaatgtt aatgtaactt ttgtttattt tgtgtactga | 2811 |
| cataatagag acttggcacc atttatttat ttttcttgat ttttggatca aattctaaaa | 2871 |
| ataaagttgc ctgttgcgaa aaaaaaaaaa aaaaaaaaaa aaaa | 2915 |

Rattus norvegicus NELL1 amino acid sequence (SEQ ID NO: 12)
Met Pro Met Asp Val Ile Leu Val Leu Trp Phe Cys Val Cys Thr Ala
Arg Thr Val Leu Gly Phe Gly Met Asp Pro Asp Leu Gln Leu Asp Ile
Ile Ser Glu Leu Asp Leu Val Asn Thr Thr Leu Gly Val Thr Gln Val
Ala Gly Leu His Asn Ala Ser Lys Ala Phe Leu Phe Gln Asp Val Gln
Arg Glu Ile His Ser Ala Pro His Val Ser Glu Lys Leu Ile Gln Leu
Phe Arg Asn Lys Ser Glu Phe Thr Phe Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Pro Arg Glu Glu Ile Arg Tyr His
Tyr Ile His Gly Gly Lys Pro Arg Thr Glu Ala Leu Pro Tyr Arg Met
Ala Asp Gly Gln Trp His Lys Val Ala Leu Ser Val Ser Ala Ser His
Leu Leu Leu His Ile Asp Cys Asn Arg Ile Tyr Glu Arg Val Ile Asp
Pro Pro Glu Thr Asn Leu Pro Pro Gly Ser Asn Leu Trp Leu Gly Gln
Arg Asn Gln Lys His Gly Phe Phe Lys Gly Ile Ile Gln Asp Gly Lys
Ile Ile Phe Met Pro Asn Gly Phe Ile Thr Gln Cys Pro Asn Leu Asn
Arg Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Gly Gln Leu Glu Asn Cys His Cys Glu Lys Thr
Cys Gln Val Ser Gly Leu Leu Tyr Arg Asp Gln Asp Ser Trp Val Asp
Gly Asp Asn Cys Gly Asn Cys Thr Cys Lys Ser Gly Ala Val Glu Cys
Arg Arg Met Ser Cys Pro Pro Leu Asn Cys Ser Pro Asp Ser Leu Pro
Val His Ile Ser Gly Gln Cys Cys Lys Val Cys Arg Pro Lys Cys Ile
Tyr Gly Gly Lys Val Leu Ala Glu Gly Gln Arg Ile Leu Thr Lys Thr
Cys Arg Glu Cys Arg Gly Gly Val Leu Val Lys Ile Thr Glu Ala Cys
Pro Pro Leu Asn Cys Ser Ala Lys Asp His Ile Leu Pro Glu Asn Gln
Cys Cys Arg Val Cys Pro Gly His Asn Phe Cys Ala Glu Ala Pro Lys
Cys Gly Glu Asn Ser Glu Cys Lys Asn Trp Asn Thr Lys Ala Thr Cys
Glu Cys Lys Asn Gly Tyr Ile Ser Val Gln Gly Asn Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Fro
Gly Tyr Ile Arg Val Asp Asp Phe Ser Cys Thr Glu His Asp Asp Cys
Gly Ser Gly Gln His Asn Cys Asp Lys Asn Ala Ile Cys Thr Asn Thr
Val Gln Gly His Ser Cys Thr Cys Gln Pro Gly Tyr Val Gly Asn Gly
Thr Ile Cys Lys Ala Phe Cys Glu Glu Gly Cys Arg Tyr Gly Gly Thr
Cys Val Ala Pro Asn Lys Cys Val Cys Pro Ser Gly Phe Thr Gly Ser
His Cys Glu Lys Asp Ile Asp Glu Cys Ala Glu Gly Phe Val Glu Cys
His Asn Tyr Ser Arg Cys Val Asn Leu Pro Gly Trp Tyr His Cys Glu
Cys Arg Ser Gly Phe His Asp Asp Gly Thr Tyr Ser Leu Ser Gly Glu
Ser Cys Ile Asp Ile Asp Glu Cys Ala Leu Arg Thr His Thr Cys Trp
Asn Asp Ser Ala Cys Ile Asn Leu Ala gly Gly Phe Asp Cys Leu Cys
Pro Ser Gly Pro Ser Cys Ser Gly Asp Cys Pro His glu Gly Gly Leu
Lys His Asn Gly Gln Val Trp Ile Leu Arg Glu Asp Arg cys Ser Val
Cys Ser Cys Lys Asp Gly Lys Ile Phe Cys Arg Arg Thr Ala Cys Asp
Cys Gln Asn Pro Asn Val Asp Leu Phe Cys Cys Pro Glu Cys Asp Thr
Arg Val Thr Ser Gln Cys Leu Asp Gln Ser Gly Gln Lys Leu Tyr Arg
Ser Gly Asp Asn Trp Thr His Ser Cys Gln Gln Cys Arg Cys Leu Glu
Gly Glu Ala Asp Cys Trp Pro Leu Ala Cys Pro Ser Leu Gly Cys Glu
Tyr Thr Ala Met Phe Glu Gly Glu Cys Cys Pro Arg Cys Val Ser Asp
Pro Cys Leu Ala Gly Asn Ile Ala Tyr Asp Ile Arg Lys Thr Cys Leu
Asp Ser Phe Gly Val Ser Arg Leu Ser Gly Ala Val Trp Thr Met Ala
Gly Ser Pro Cys Thr Thr Cys Lys Cys Lys Asn Tly Arg Val Cys Cys
Ser Val Asp Leu Glu Cys Ile Glu Asn Asn
Felis catus NELL1 isoform l amino acid sequence (SEQ ID NO: 13)
Met Pro Arg Asp Val Ile Leu Val Val Trp Phe Cys Val Cys Thr Ala
Arg Thr Val Val Gly Phe Gly Thr Asp Pro Asp Leu Gln Val Asp Ile
Ile Ala Glu Leu Asp Leu Val Asn Thr Thr Ala Gly Val Thr Gln Val
Ser Gly Leu His Asn Ala Ser Lys Ala Tyr Leu Phe Gln Glu Thr Glu
Arg Glu Ile His Ala Ala Pro His Val Ser Glu Lys Leu Ile Gln Leu
Phe Arg Asn Lys Ser Glu Phe Ser Phe Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Leu Arg Asp Glu Ile Arg Tyr His
Tyr Ile His Asn Gly Lys Pro Arg Thr Glu Ala Leu Pro Tyr Arg Met
Ala Asp Gly Gln Trp His Lys Val Ala Leu Ser Ile Ser Ala Ser His
Leu Leu Leu His Val Asp Cys Asn Arg Ile Tyr Glu Arg Val Ile Asp
Pro Pro Glu Thr Asn Leu Pro Pro Gly Ser Asn Val Trp Leu Gly Gln
Arg Asn Gln Lys His Gly Leu Phe Lys Gly Ile Ile Gln Asp Gly Lys
Ile Ile Phe Met Pro Asn Gly Tyr Ile Thr Gln Cys Pro Asn Leu Asn
Arg Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Asn Gln Leu Glu Asn Cys His Cys Glu Lys Thr
Cys Gln Val Ser Gly Leu Leu Tyr Arg Asp Gln Asp Ser Trp Val Asp
Gly Asp His Cys Arg Asn Cys Thr Cys Lys Ser Gly Ala Val Glu Cys
Arg Arg Met Ser Cys Pro Pro Leu Asn Cys Ser Pro Asp Ser Leu Pro
Val His Ile Ala Gly Gln Cys Cys Lys Val Cys Arg Pro Lys Cys Ile
Tyr Gly Gly Lys Val Leu Ala Glu Gly Gln Arg Ile Leu Thr Lys Ser
Cys Arg Glu Cys Arg Gly Gly Val Leu Val Lys Ile Thr Asp Ala Cys
Pro Pro Leu Asn Cys Ser Glu Lys Asp His Ile Leu Pro Glu Asn Gln
Cys Cys Ser Val Cys Arg Gly His Asn Phe Cys Ala Glu Gly Pro Thr
Cys Gly Glu Asn Ser Glu Cys Lys Asn Trp Asn Thr Lys Ala Thr Cys
Glu Cys Lys Asn Gly Tyr Ile Ser Val Gln Gly Asp Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Pro
Gly Tyr Ile Arg Val Asp Asp Phe Ser Cys Thr Glu His Asp Glu Cys
Gly Ser Gly Gln His Asn Cys Asp Glu Asn Ala Ile Cys Thr Asn Thr
Val Gln Gly His Ser Cys Thr Cys Lys Pro Gly Tyr Val Gly Asn Gly
Thr Ile Cys Arg Ala Phe Cys Gln Glu Gly Cys Arg Tyr Gly Gly Thr
Cys Val Ser Pro Asn Lys Cys Val Cys Pro Ser Gly Phe Thr Gly Ser
His Cys Glu Lys Asp Ile Asp Glu Cys Thr Glu Gly Ile Ile Glu Cys
His Asn His Ser Arg Cys Val Asn Leu Pro Gly Trp Tyr His Cys Glu
Cys Arg Ser Gly Phe His Asp Asp Gly Thr Tyr Ser Leu Ser Gly Glu
Ser Cys Ile Asp Ile Asp Glu Cys Ala Leu Arg Thr His Thr Cys Trp
Asn Asp Ser Ala Cys Ile Asn Leu Ala Gly Gly Phe Asp Cys Leu Cys
Pro Ser Gly Pro Ser Cys Ser Gly Asp Cys Pro His Glu Gly Gly Leu
Lys Arg Asn Gly Gln Val Trp Thr Leu Lys Glu Asp Arg Cys Ser Val
Cys Ser Cys Lys Asp Gly Lys Ile Phe Cys Arg Arg Thr Ala Cys Asp
Cys Gln Asn Pro Ser Val Asp Leu Phe Cys Cys Pro Glu Cys Asp Thr
Arg Val Thr Ser Gln Cvs Leu Asp Gln Asn Gly His Lys Leu Tyr Arg
Ser Gly Asp Asn Trp Thr His Ser Cys Gln Gln Cys Arg Cys Leu Glu
Gly Glu Val Asp Cys Trp Pro Leu thr Cys Pro Asn Leu Ser Cys Glu
Tyr Thr Ala Met Leu Glu Gly Glu Cys Cys Pro Arg Cys Val Ser Asp
Pro Cys Leu Ala Asp Asn Ile Ala Tyr Asp Ile Arg Lys Thr Cys Leu
Asp Ser Tyr Gly Ile Ser Arg Leu Ser Gly Ala Val Trp Thr Met Ala
Gly Ser Pro Cys Thr Thr Cys Lys Cys Lys Asn Gly Ser Val Cys Cys
Ser Val Asp Leu Glu Cys Leu His Asn Asn
Felis catus NELL1 isoform 2 amino acid sequence (SEQ ID NO: 14)
Met Pro Arg Asp Val Ile Leu Val Val Trp Phe Cys Val Cys Thr Ala
Arg Thr Val Val Gly Phe Gly Thr Asp Pro Asp Leu Gln Val Asp Ile
Ile Ala Glu Leu Asp Leu Val Asn Thr Thr Ala Gly Val Thr Gln Val
Ser Gly Leu His Asn Ala Ser Lys Ala Tyr Leu Phe Gln Glu Thr Glu
Arg Glu Ile His Ala Ala Pro His Val Ser Glu Lys Leu Ile Gln Leu
Phe Arg Asn Lys Ser Glu Phe Ser Phe Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Leu Arg Asp Glu Ile Arg Tyr His
Tyr Ile His Asn Gly Lys Pro Arg Thr Glu Ala Leu Pro Tyr Arg Met
Ala Asp Gly Gln Trp His Lys Val Ala Leu Ser Ile Ser Ala Ser His
Leu Leu Leu His Val Asp Cys Asn Arg Ile Tyr Glu Arg Val Ile Asp
Pro Pro Glu Thr Asn Leu Pro Pro Gly Ser Asn Val Trp Leu Gly Gln
Arg Asn Gln Lys His Gly Leu Phe Lys Gly Ile Ile Gln Asp Gly Lys
Ile Ile Phe Met Pro Asn Gly Tyr Ile Thr Gln Cys Pro Asn Leu Asn
Arg Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Asn Gln Leu Glu Asn Cys His Cys Glu Lys Thr
Cys Gln Val Ser Gly Leu Leu Tyr Arg Asp Gln Asp Ser Trp Val Asp
Gly Asp His Cys Arg Asn Cys Thr Cys Lys Ser Gly Ala Val Glu Cys
Arg Arg Met Ser Cys Pro Pro Leu Asn Cys Ser Pro Asp Ser Leu Fro
Val His Ile Ala Gly Gln Cys Cys Lys Val Cys Arg Pro Lys Cys Ile
Tyr Gly Gly Lys Val Leu Ala Glu Gly Gln Arg Ile Leu Thr Lys Ser
Cys Arg Glu Cys Arg Gly Gly Val Leu Val Lys Ile Thr Asp Ala Cys
Pro Pro Leu Asn Cys Ser Glu Lys Asp His Ile Leu Pro Glu Asn Gln
Cys Cys Ser Val Cys Arg Gly His Asn Phe Cys Ala Glu Gly Pro Thr
Cys Gly Glu Asn Ser Glu Cys Lys Asn Trp Asn Thr Lys Ala Thr Cys
Glu Cys Lys Asn Gly Tyr Ile Ser Val Gln Gly Asp Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Pro
Gly Tyr Ile Arg Val Asp Asp Phe Ser Cys Thr Glu His Asp Glu Cys
Gly Ser Gly Gln His Asn Cys Asp Glu Asn Ala Ile Cys Thr Asn Thr
Val Gln Gly His Ser Cys Thr Cys Lys Pro Gly Tyr Val Gly Asn Gly
Thr Ile Cys Arg Ala Phe Cys Gln Glu Gly Cys Arg Tyr Gly Gly Thr
Cys Val Ser Pro Asn Lys Cys Val Cys Pro Ser Gly Phe Thr Gly Ser
His Cys Glu Lys Asp Ile Asp Glu Cys Ala Leu Arg Thr His Thr Cys
Trp Asn Asp Ser Ala Cys Ile Asn Leu Ala Gly Gly Phe Asp Cys Leu
Cys Pro Ser Gly Pro Ser Cys Ser Gly Asp Cys Pro His Glu Gly Gly
Leu Lys Arg Asn Gly Gln Val Trp Thr Leu Lys Glu Asp Arg Cys Ser
Val Cys Ser Cys Lys Asp Gly Lys Ile Phe Cys Arg Arg Thr Ala Cys
Asp Cys Gln Asn Pro Ser Val Asp Leu Phe Cys Cys Pro Glu Cys Asp
Thr Arg Val Thr Ser Gln Cys Leu Asp Gln Asn Gly His Lys Leu Tyr
Arg Ser Gly Asp Asn Trp Thr His Ser Cys Gln Gln Cys Arg Cys Leu
Glu Gly Glu Val Asp Cys Trp Pro Leu Thr Cys Pro Asn Leu Ser Cys
Glu Tyr Thr Ala Met Leu Glu Gly Glu Cys Cys Pro Arg Cys Val Ser
Asp Pro Cys Leu Ala Asp Asn Ile Ala Tyr Asp Ile Arg Lys Thr Cys
Leu Asp Ser Tyr Gly Ile Ser Arg Leu Ser Gly Ala Val Trp Thr Met
Ala Gly Ser Pro Cys Thr Thr Cys Lys Cys Lys Asn Gly Ser Val Cys
Cys Ser Val Asp Leu Glu Cys Leu His Asn Asn
Canis lupis familiaris NELL1 amino acid sequence (SEP ID NO: 15)
Met Thr Ser Thr Ser Phe Leu Leu Trp Leu Gly Cys Val His Asn Thr
Lys Phe Pro Phe Pro Leu Val Leu Val Thr Arg Ala Ile Val Val Val
Val Val Glu Val Val Gly Val Gly Ser Pro Gly Val Arg Ile Arg Ser
Thr Gly Cys Asp Ile Leu Leu Leu Tyr Glu Val Leu Glu His Leu Leu
Gly Ile Arg Phe Leu Cys Val Asp Gln Gly Glu Asn Ser Cys His His
Gly Gln Cys Ala Cys Arg Leu Gln Val Ile Val Pro Lys Ala Leu Met
Ser Val Phe Glu Ala Lys Thr Ala Val Cys Phe Phe Pro Val Val Gly
Phe Gly Thr Asp Pro Asp Leu Gln Met Asp Ile Ile Thr Glu Leu Asp
Leu Val Asn Ile Ser Leu Gly Val Thr Gln Val Ser Gly Leu His Asn
Ala Ser Lys Ala Tyr Val Phe Gln Asp Thr Ala Arg Glu Ile His Ala
Ala Pro His Val Ser Glu Lys Leu Ile Gln Leu Phe Arg Asn Lys Ser
Asp Phe Thr Phe Leu Ala Thr Val Gln Gln Lys Pro Ser Thr Ser Gly
Val Ile Leu Ser Ile Arg Glu Leu Glu His Ser Tyr Phe Glu Leu Glu
Ser Ser Gly Leu Arg Asp Glu Ile Arg Tyr His Tyr Met His Asn Gly
Lys Pro Arg Thr Glu Ala Leu Pro Tyr Arg Leu Ala Asp Gly Gln Trp
His Lys Val Ala Leu Ser Val Ser Ala Ser His Leu Leu Leu His Ile
Asp Cys Asn Arg Ile Tyr Glu Arg Val Ile Asp Pro Pro Glu Thr Asn
Leu Pro Pro Gly Ser Asn Leu Trp Leu Gly Gln Arg Asn Gln Lys His
Gly Phe Phe Lys Gly Ile Ile Gln Asp Gly Lys Ile Ile Phe Met Pro
Asn Gly Tyr Ile Thr Gln Cys Pro Asn Leu Asn Arg Thr Cys Pro Thr
Cys Ser Asp Phe Leu Ser Leu Val Gln Gly Ile Met Asp Leu Gln Glu
Leu Leu Ala Lys Met Thr Ala Lys Leu Asn Tyr Ala Glu Thr Arg Leu
Ser Gln Leu Glu Asn Cys His Cys Glu Lys Thr Cys Gln Val Ser Gly
Leu Leu Tyr Arg Asp Gln Asp Ser Trp Val Asp Gly Asp His Cys Arg
Asn Cys Thr Cys Lys Gly Gly Ala Val Glu Cys Arg Arg Met Ser Cys
Pro Pro Leu Asn Cys Ser Pro Asp Ser Leu Pro Val His Ile Ala Gly
Gln Cys Cys Lys Val Cys Arg Pro Lys Cys Ile Tyr Gly Gly Arg Val
Leu Ala Glu Gly Gln Arg Ile Leu Thr Lys Ser Cys Arg Glu Cys Arg
Gly Gly Val Leu Val Lys Ile Thr Asp Ala Cys Pro Pro Leu Asn Cys
Ser Glu Lys Asp His Ile Leu Pro Glu Asn Gln Cys Cys Ser Val Cys
Arg Gly His Asn Phe Cys Ala Glu Gly Pro Lys Cys Gly Glu Asn Ser
Glu Cys Lys Asn Trp Asn Thr Lys Ala Thr Cys Glu Cys Lys Asn Gly
Tyr Ile Ser Val Gln Gly Asp Ser Ala Tyr Cys Glu Asp Ile Asp Glu
Cys Ala Ala Lys Met His Tyr Cys His Ala Asn Thr Val Cys Val Asn
Leu Pro Gly Leu Tyr Arg Cys Asp Cys Val Pro Gly Tyr Ile Arg Val
Asp Asp Phe Ser Cys Thr Glu His Asp Glu Cys Gly Ser Gly Gln His
Asn Cys Asp Glu Asn Ala Ile Cys Thr Asn Thr Val Arg Gly His Ser
Cys Thr Cys Lys Pro Gly Tyr Val Gly Asn Gly Thr Ile Cys Arg Ala
Phe Cys Gln Glu Gly Cys Arg Tyr Gly Gly Ser Cys Val Ser Pro Asn
Lys Cys Val Cys Pro Ser Gly Phe Thr Gly Ser His Cys Glu Lys Asp
Ile Asp Glu Cys Thr Glu Gly Ile Ile Glu Cys His Asn His Ser Arg
Cys Val Asn Leu Pro Gly Trp Tyr His Cys Glu Cys Arg Ser Gly Phe
His Asp Asp Gly Thr Tyr Ser Leu Ser Gly Glu Ser Cys Ile Asp Ile
Asp Glu Cys Ala Leu Arg Thr His Thr Cys Trp Asn Asp Ser Ala Cys
Ile Asn Leu Ala Gly Gly Phe Asp Cys Leu Cys Pro Ser Gly Pro Ser
Cys Ser Gly Asp Cys Pro His Glu Gly Gly Leu Lys Arg Asn Gly Gln
Val Trp Thr Leu Lys Glu Asp Arg Cys Ser Val Cys Ser Cys Lys Asp
Gly Lys Ile Leu Cys Arg Arg Thr Ala Cys Asp Cys Gln Asn Pro Ser
Val Asp Leu Phe Cys Cys Pro Glu Cys Asp Thr Arg Val Thr Ser Gln
Cys Leu Asp Gln Asn Gly His Lys Leu Tyr Arg Ser Gly Asp Asn Trp
Thr His Ser Cys Gln Gln Cys Arg Cys Leu Glu Gly Glu Val Asp Cys
Trp Pro Leu Thr Cys Pro Asn Leu Ser Cys Glu Tyr Thr Ala Ile Leu
Glu Gly Glu Cys Cys Pro Arg Cys Val Ser Asp Pro Cys Leu Ala Asp
Asn Ile Ala Tyr Asp Ile Arg Lys Thr Cys Leu Asp Ser Tyr Gly Ile
Ser Arg Leu Ser Gly Ser Val Trp Thr Met Ala Gly Ser Pro Cys Thr
Thr Cys Lys Cys Lys Asn Gly Ser Val Cys Cys Ser Val Asp Leu Glu
Cys Leu His Asn Asn
Ovis aries NELL1 amino acid sequence (SEQ ID NO: 16)
Met Pro Arg Gly Val Ile Leu Val Val Cys Phe Cys Val Cys Ala Ala
Arg Thr Val Val Gly Phe Gly Met Asp Pro Asp Leu Gln Leu Asp Ile
Ile Thr Glu Leu Asp Leu Val Asn Thr Thr Leu Gly Val Thr Gln Val
Ser Gly Leu His Asn Thr Ser Lys Ala Phe Leu Phe Gln Asp Ala Glu
Arg Glu Ile His Ala Ala Pro His Val Ser Glu Lys Leu Ile Gln Leu
Phe Arg Asn Lys Ser Glu Phe Thr Phe Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Leu Arg Asp Glu Ile Arg Tyr His
Tyr Met His Ser Gly Arg Pro Arg Thr Glu Ala Leu Pro Tyr Arg Leu
Ala Asp Gly Gln Trp His Arg Val Ala Leu Ser Val Ser Ala Ser His
Leu Leu Leu His Ile Asp Cys Asn Arg Ile Tyr Glu Arg Val Ile Asp
Pro Pro Glu Thr Asn Leu Pro Pro Gly Ser Asn Leu Trp Leu Gly Gln
Arg Asn Gln Lys His Gly Leu Phe Lys Gly Ile Ile Gln Asp Gly Lys
Ile Ile Phe Met Pro Asn Gly Tyr Ile Thr Gln Cys Pro Asn Leu Asn
Arg Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Ser Gln Leu Glu Asn Cys His Cys Glu Lys Thr
Cys Gln Val Ser Gly Leu Leu Tyr Arg Asp Gln Asp Ser Trp Val Asp
Gly Asp His Cys Arg Asn Cys Thr Cys Lys Ser Gly Ala Val Glu Cys
Arg Arg Met Ser Cys Pro Pro Leu Asn Cys Ser Pro Asp Ser Leu Pro
Val His Ile Ala Gly Gln Cys Cys Lys Val Cys Arg Pro Lys Cys Ile
Tyr Gly Gly Lys Val Leu Ala Glu Gly Gln Arg Ile Leu Ser Lys Asn
Cys Gln Glu Cys Arg Gly Gly Val Leu Val Lys Ile Thr Glu Ala Cys
Pro Leu Leu Asn Cys Ser Glu Lys Asp His Ile Leu Pro Glu Asn Gln
Cys Cys Ser Val Cys Arg Gly His Asn Phe Cys Ala Glu Gly Pro Lys
Cys Gly Glu Asn Ser Glu Cys Lys Asn Trp Asn Thr Lys Ala Thr Cys
Glu Cys Lys Asn Gly Tyr Ile Ser Val Gln Gly Asp Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Pro
Gly Tyr Ile Arg Val Asp Asp Phe Ser Cys Thr Glu His Asp Asp Cys
Gly Ser Gly Gln His Asn Cys Asp Glu Asn Ala Ile Cys Thr Asn Thr
Val Gln Gly His Ser Cys Thr Cys Lys Pro Gly Tyr Val Gly Asn Gly
Thr Ile Cys Arg Ala Phe Cys Glu Glu Gly Cys Arg Tyr Gly Gly Thr
Cys Met Ala Pro Asn Lys Cys Val Cys Pro Ser Gly Phe Thr Gly Ser
His Cys Glu Lys Asp Ile Asp Glu Cys Ala Glu Gly Ile Ile Glu Cys
His Ser His Ser Arg Cys Val Asn Leu Pro Gly Trp Tyr His Cys Glu
Cys Arg Ser Gly Phe His Asp Asp Gly Thr Tyr Ser Leu Ser Gly Glu
Ser Cys Val Asp Ile Asp Glu Cys Ala Leu Arg Thr His Thr Cys Trp
Asn Asp Ser Ala Cys Ile Asn Leu Ala Gly Gly Phe Asp Cys Leu Cys
Pro Ser Gly Pro Ser Cys Ser Gly Asp Cys Pro His Glu Gly Gly Leu
Lys Arg Asn Gly Gln Val Trp Thr Leu Lys Glu Asp Arg Cys Ser Val
Cys Ser Cys Lys Asp Gly Lys Ile Phe Cys Arg Arg Thr Ala Cys Asp
Cys Gln Asn Pro Ser Val Asp Leu Phe Cys Cys Pro Glu Cys Asp Thr
Arg Val Thr Ser Gln Cys Leu Asp Gln Asn Gly Asn Lys Leu Tyr Arg
Ser Gly Asp Asn Trp Thr His Ser Cys Gln Gln Cys Arg Cys Leu Glu
Gly Glu Val Asp Cys Trp Pro Leu Thr Cys Pro Ser Leu Ser Cys Glu
Tyr Thr Thr Ile Leu Glu Glu Glu Cys Cys Pro Arg Cys Val Ser Asp
Pro Cys Leu Ala Asp Asn Ile Ala Tyr Asp Ile Arg Lys Thr Cys Leu
Asp Ser Tyr Gly Leu Ser Arg Leu Ser Gly Ser Val Trp Thr Met Ala
Gly Ser Pro Cys Thr Thr Cys Lys Cys Lys Asn Gly Ser Val cys Cys
Ser Val Asp Leu Glu Cys Leu His Asn Asn
Homo sapiens NELL1 fragment amino acid sequence (SEQ ID NO: 17)
Phe Gly Met Asp Pro Asp Leu Gln Met Asp Ile
Val Thr Glu Leu Asp Leu Val Asn Thr Thr Leu Gly Val Ala Gln Val
Ser Gly Met His Asn Ala Ser Lys Ala Phe Leu Phe Gln Asp Ile Glu
Arg Glu Ile His Ala Ala Pro His Val Ser Glu Lys Leu Ile Gln Leu
Phe Arg Asn Lys Ser Glu Phe Thr Ile Leu Ala Thr Val Gln Gln Lys
Pro Ser Thr Ser Gly Val Ile Leu Ser Ile Arg Glu Leu Glu His Ser
Tyr Phe Glu Leu Glu Ser Ser Gly Leu Arg Asp Glu Ile Arg Tyr His
Tyr Ile His Asn Gly Lys Pro Arg Thr Glu Ala Leu Pro Tyr Arg Met
Ala Asp Gly Gln Trp His Lys Val Ala Leu Ser Val Ser Ala Ser His
Leu Leu Leu His Val Asp Cys Asn Arg Ile Tyr Glu Arg Val Ile Asp
Pro Pro Asp Thr Asn Leu Pro Pro Gly Ile Asn Leu Trp Leu Gly Gln
Arg Asn Gln Lys His Gly Leu Phe Lys Gly Ile Ile Gln Asp Gly Lys
Ile Ile Phe Met Pro Asn Gly Tyr Ile Thr Gln Cys Tro Asn Leu Asn
His Thr Cys Pro Thr Cys Ser Asp Phe Leu Ser Leu Val Gln Gly Ile
Met Asp Leu Gln Glu Leu Leu Ala Lys Met Thr Ala Lys Leu Asn Tyr
Ala Glu Thr Arg Leu Ser Gln Leu Glu Asn Cys His Cys Glu Lys Thr
Cys Gln Val Ser Gly Leu Leu Tyr Arg Asp Gln Asp Ser Trp Val Asp
Gly Asp His Cys Arg Asn Cys Thr Cys Lys Ser Gly Ala Val Glu Cys
Arg Arg Met Ser Cys Pro Pro Leu Asn Cys Ser Pro Asp Ser Leu Pro
Val His Ile Ala Gly Gln Cys Cys Lys Val Cys Arg Pro Lys Cys Ile
Tyr Gly Gly Lys Val Leu Ala Glu Gly Gln Arg Ile Leu Thr Lys Ser
Cys Arg Glu Cys Arg Gly Gly Val Leu Val Lys Ile Thr Glu Met Cys
Pro Pro Leu Asn Cys Ser Glu Lys Asp His Ile Leu Pro Glu Asn Gln
Cys Cys Arg Val Cys Arg Gly His Asn Phe Cys Ala Glu Gly Pro Lys
Cys Gly Glu Asn Ser Glu Cys Lys Asn Trp Asn Thr Lys Ala Thr Cys
Glu Cys Lys Ser Gly Tyr Ile Ser Val Gln Gly Asp Ser Ala Tyr Cys
Glu Asp Ile Asp Glu Cys Ala Ala Lys Met His Tyr Cys His Ala Asn
Thr Val Cys Val Asn Leu Pro Gly Leu Tyr Arg Cys Asp Cys Val Pro
Gly Tyr Ile Arg Val Asp Asp Phe Ser Cys Thr Glu His Asp Glu Cys
Gly Ser Gly Gln His Asn Cys Asp Glu Asn Ala Ile Cys Thr Asn Thr
Val Gln Gly His Ser Cys Thr Cys Lys Pro Gly Tyr Val Gly Asn Gly
Thr Ile Cys Arg Ala Phe Cys Glu Glu Gly Cys Arg Tyr Gly Gly Thr
Cys Val Ala Pro Asn Lys Cys Val Cys Pro Ser Gly Phe Thr Gly Ser
His Cys Glu Lys Asp Ile Asp Glu Cys Ser Glu Gly Ile Ile Glu Cys
His Asn His Ser Arg Cys Val Asn Leu Pro Gly Trp Tyr His Cys Glu
Cys Arg Ser Gly Phe His Asp Asp Gly Thr Tyr Ser Leu Ser Gly Glu
Ser Cys Ile Asp Ile Asp Glu Cys Ala Leu Arg Thr His Thr Cys Trp
Asn Asp Ser Ala Cys Ile Asn Leu Ala Gly Gly Phe Asp Cys Leu Cys
Pro Ser Gly Pro Ser Cys Ser
Equus caballus NELL1 fragment amino acid seauence (SEQ ID NO: 18)
Phe Gly Met Asp Pro Asp Leu Gln Met Asp Ile Ile Thr Glu
Leu Asp Leu Val Asn Thr Thr Leu Gly Val Thr Gln Val Ser Gly Leu
His Asn Ala Ser Lys Ala Phe Leu Phe Gln Asp Val Glu Arg Glu Ile
His Ala Ala Pro His Val Ser Glu Lys Leu Ile Gln Leu Phe Arg Asn
Lys Ser Glu Phe Thr Phe Leu Ala Thr Val Gln Gln Lys Pro Ser Thr
Ser Gly Val Ile Leu Ser Ile Arg Glu Leu Glu Asn Ser Tyr Phe Glu
Leu Glu Ser Ser Gly Leu Arg Asp Glu Ile Arg Tyr His Tyr Thr His
Lys Gly Lys Pro Arg Thr Glu Ala Leu Pro Tyr Arg Met Ala Asp Gly
Arg Trp His Lys Val Ala Leu Ser Val Ser Ala Ser His Leu Leu Leu
His Ile Asp Cys Asn Arg Ile Tyr Glu Arg Val Ile Asp Thr Pro Glu
Thr Asn Leu Pro Pro Gly Ser Asn Leu Trp Leu Gly Gln Arg Asn Gln
Lys His Gly Leu Phe Lys Gly Ile Ile Gln Asp Gly Lys Ile Ile Phe
Met Pro Asn Gly Tyr Ile Thr Gln Cys Pro Asn Leu Asn Arg Thr Cys
Pro Thr Cys Ser Asp Phe Leu Ser Leu Val Gln Gly Ile Met Asp Leu
Gln Glu Leu Leu Ala Lys Met Thr Ala Lys Leu Asn Tyr Ala Glu Thr
Arg Leu Ser Gln Leu Glu Asn Cys His Cys Glu Lys Thr Cys Gln Val
Ser Gly Leu Leu Tyr Arg Asp Gln Asp Ser Trp Val Asp Gly Asp His
Cys Arg Asn Cys Thr Cys Lys Ser Gly Ala Val Glu Cys Arg Arg Met
Ser Cys Pro Pro Leu Asn Cys Ser Pro Asp Ser Leu Pro Val His Val
Ala Gly Gln Cys Cys Lys Val Cys Arg Pro Lys Cys Ile Tyr Gly Gly
Lys Val Leu Ala Glu Gly Gln Arg Ile Leu Thr Lys Ser Cys Arg Glu
Cys Arg Gly Gly Val Leu Val Lys Ile Thr Glu Ala Cys Pro Pro Leu
Asn Cys Ser Asp Lys Asp His Ile Leu Pro Glu Asn Gln Cys Cys Ser
Val Cys Arg Gly His Asn Phe Cys Ala Glu Gly Pro Lys Cys Gly Glu
Asn Ser Glu Cys Lys Asn Trp Asn Thr Lys Ala Thr Cys Glu Cys Lys
Asn Gly Tyr Ile Ser Val Gln Gly Asp Ser Ala Tyr Cys Glu Asp Ile
Asp Glu Cys Ala Ala Lys Met His Tyr Cys Arg Ala Asn Thr Val Cys
Val Asn Leu Pro Gly Leu Tyr Arg Cys Asp Cys Val Pro Gly Tyr Ile
Arg Val Asp Asp Phe Ser Cys Thr Glu His Asp Glu Cys Gly Ser Gly
Gln His Asn Cys Asp Glu Asn Ala Ile Cys Thr Asn Thr Val Gln Gly
His Ser Cys Thr Cys Lys Pro Gly Tyr Val Gly Asn Gly Thr Ser Cys
Arg Ala Phe Cys Glu Glu Gly Cys Arg Tyr Gly Gly Thr Cys Val Ala
Pro Asn Lys Cys Val Cys Pro Ser Gly Phe Thr Gly Ser His Cys Glu
Lys Asp Ile Asp Glu Cys Thr Glu Gly Ile Ile Glu Cys His Asn His
Ser Arg Cys Val Asn Leu Pro Gly Trp Tyr His Cys Glu Cys Arg Ser
Gly Phe His Asp Asp Gly Thr Tyr Ser Leu Ser Gly Glu Ser Cys Ile
Asp Ile Asp Glu Cys Ala Leu Arg Thr His Thr Cys Trp Asn Asp Ser
Ala Cys Ile Asn Leu Ala Gly Gly Phe Asp Cys Leu Cys Pro Ser Gly
Pro Ser Cys Ser
Bos taurus NELL1 amino acid sequence (SEQ ID NO: 19)
Met Ala Leu Cys Ser Phe Ser Val Val Gly Phe Gly Leu Asp Pro Asp
Leu Gln Leu Asp Ile Ile Thr Glu Leu Asp Leu Val Asn Thr Thr Leu
Gly Val Thr Gln Val Ser Gly Leu His Asn Thr Ser Lys Ala Phe Leu
Phe Gln Asp Ala Glu Arg Glu Ile His Ala Ala Pro His Val Ser Glu
Lys Leu Ile Gln Leu Phe Arg Asn Lys Ser Glu Phe Thr Phe Leu Ala
Thr Val Gln Gln Lys Pro Ser Thr Ser Gly Val Ile Leu Ser Ile Arg
Glu Leu Glu His Ser Tyr Phe Glu Leu Glu Ser Ser Gly Leu Arg Asp
Glu Ile Arg Tyr His Tyr Val His Ser Gly Arg Pro Arg Thr Glu Ala
Leu Pro Tyr Arg Leu Ala Asp Gly Gln Trp His Arg Val Ala Leu Ser
Val Ser Ala Ser His Leu Leu Leu His Ile Asp Cys Asn Arg Ile Tyr
Glu Arg Val Ile Asp Pro Pro Glu Thr Asn Leu Pro Pro Gly Ser Asn
Leu Trp Leu Gly Gln Arg Asn Gln Lys His Gly Leu Phe Lys Gly Ile
Ile Gln Asp Gly Lys Ile Ile Phe Met Pro Asn Gly Tyr Ile Thr Gln
Cys Pro Asn Leu Asn Arg Thr Cys Pro Thr Cys Ser Asp Phe Leu Ser
Leu Val Gln Gly Ile Met Asp Leu Gln Glu Leu Leu Ala Lys Met Thr
Ala Lys Leu Asn Tyr Ala Glu Thr Arg Leu Ser Gln Leu Glu Asn Cys
His Cys Glu Lys Thr Cys Gln Val Ser Gly Leu Leu Tyr Arg Asp Gln
Asp Ser Trp Val Asp Gly Asp His Cys Arg Asn Cys Thr Cys Lys Ser
Gly Ala Val Glu Cys Arg Arg Met Ser Cys Pro Pro Leu Asn Cys Ser
Pro Asp Ser Leu Pro Val His Ile Ala Gly Glu Cys Cys Lys Val Cys
Arg Pro Lys Cys Ile Tyr Gly Gly Lys Val Leu Ala Glu Gly Gln Arg
Ile Leu Ser Lys Ser Cys Gln Glu Cys Arg Gly Gly Val Leu Val Lys
Ile Thr Glu Ala Cys Pro Leu Leu Asn Cys Ser Glu Lys Asp His Ile
Leu Pro Glu Asn Gln Cys Cys Ser Val Cys Arg Gly His Asn Phe Cys
Ala Glu Gly Pro Lys Cys Gly Glu Asn Ser Glu Cys Lys Asn Trp Asn
Thr Lys Ala Thr Cys Glu Cys Lys Asn Gly Tyr Ile Ser Val Gln Gly
Asp Ser Ala Tyr Cys Glu Asp Ile Asp Glu Cys Ala Ala Lys Met His
Tyr Cys His Ala Asn Thr Val Cys Val Asn Leu Pro Gly Leu Tyr Arg
Cys Asp Cys Val Pro Gly Tyr Ile Arg Val Asp Asp Phe Ser Cys Thr
Glu His Asp Asp Cys Gly Ser Gly Gln His Asn Cys Asp Glu Asn Ala
Ile Cys Thr Asn Thr Val Gln Gly His Ser Cys Thr Cys Lys Pro Gly
Tyr Val Gly Asn Gly Thr Ile Cys Arg Gly Met Pro Glu Val Gly Fro
Pro Arg Ala Leu Leu Asn Ser Leu Asp Leu Gly Phe Leu Ser Phe Ser
Lys Glu Ala Leu Ala Val Gly Met Ile Thr Leu Glu Gly Asn Ile Val
Ala Lys Ser Phe Thr Asp Asp Glu Thr Leu Val Glu Arg Gly Arg Glu
Lys Val Ile Ala Leu Leu Phe Ser Trp Leu His Lys Glu Lys Leu Ser
Leu Glu Asn Leu Arg Asp Ile Tyr Cys Lys Ala Asn Ser Leu Val Gly
Leu Asp His Leu Pro Gln Arg

### EXAMPLES

The present invention, thus generally described, will be understood more readily by reference to the following Examples, which are provided by way of illustration and are not intended to be limiting of the instant invention. The Examples are not intended to represent that the experiments below are all experiments performed.

### Example 1

### Effects of NELL1 on an Animal Model of SARS-CoV-2 Infection

Due to the novelty of the nature, infection and effects of SARS-CoV-2, re-purposing and testing of existing therapeutics like NELL1 require direct testing on acute lung tissue injury and ARDS models, using conditions that trigger the same damage found in coronavirus-induced injuries and/or more directly using existing models (in vitro and in vivo) created for testing candidate therapeutics for coronavirus infection. In the latter, models have been generated for SARS-CoV virus that caused an epidemic in 2002-2003. Because of the great similarity in structure of SARS-CoV and SAR-CoV-2 (80%) and that they infect target cells by binding the angiotensin-converting enzyme II (ACE2) and subsequent uptake via receptor-mediated endocytosis, current models for SARS-CoV can be utilized for testing therapeutics against SARS-CoV-2.

Transgenic mice expressing the human ACE2 receptor and adaptation of SARS-CoV to mice by serial passage are also commercially available and were used to test the efficacy of NELL1 in treating virally-induced tissue damage. Coronavirus infection in these models is severe and result in lethality, just like in human patients (Gretebeck L M and Subbarao K 2015 Current Opinion in Virology 13:123-129). These studies used a commercially available mouse model using transgenic (tg) mice with the cytokeratin K18 promoter driving the high expression of the human Angiotensin Converting Enzyme 2 receptor (hACE2r tg; Jackson Laboratory, strain 034860 B6.Cg-TG(K18-ACE2) 2Primn/J. In a preliminary study, the 3-4 weeks old mice were injected via nasal injection of SARS-CoV-2 (NR-52281, BEI Resources) virus in two different concentrations: 2,800 pfu in 50 μl volume or 28,000 pfu in 50 μl volume. Sterile PBS was used as a sham control group. The body weight and clinical symptoms were monitored daily after the infection.

Mice challenged with both a high and medium dose of viral particles showed a substantial decrease in body weight starting at day 4 post infection compared to PBS-injected mice. Despite a 10-fold higher dose of viral injection, an equal number of SARS-CoV-2 particles could be identified with both the doses in lung tissue, trachea and nasal turbinate on day 4 post viral injection. However, the severe decrease in body weight immediately after the infection (4 days) suggests using the lower dose of 2800 pfu of SARS-CoV-2 for ongoing studies.

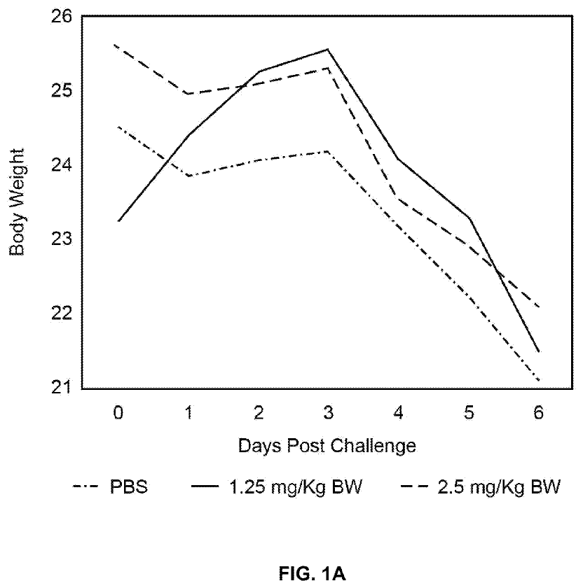

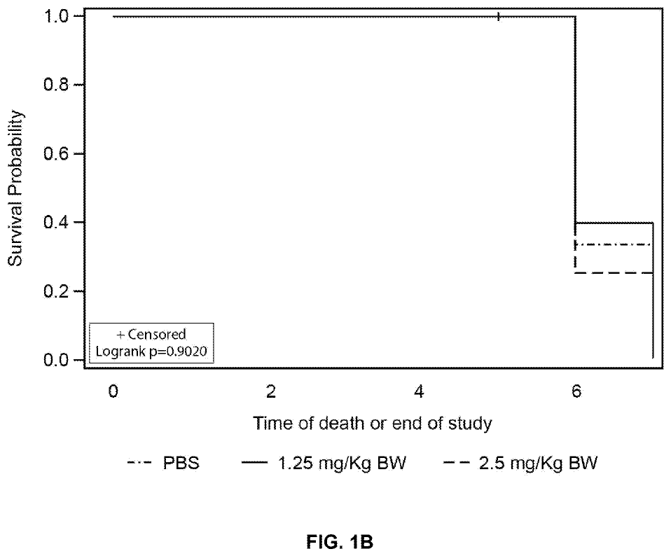

In a first pilot experiment, 10 tg-mice hACE2r (5 male+5 female) were infected by the nasal injection route with 2.8×10³ pfu of SARS-CoV-2 virus. Two doses of NELL1 protein with concentrations of 1.25 mg/kg body weight and 2.5 mg/kg body weight (BW) (in 100 μl PBS) were delivered to 5 animals each. A set of control mice (n=5) mock treated with PBS only were infected similarly. The clinical symptoms of infected control mice include abrupt loss in body weight at 1 dpi, with ruffled fur, hunched back, and reduced mobility. The administration of NELL1 proteins to SARS-CoV-2-infected tg-mice at a dose of 1.25 mg/kg (average weight t=22 gm) and administration regimen of Day0 and Day3 post-infection (p.i.) shows a significant increase in body weight (p<0.05), peaking to >10% at Day3 (FIG. 1A). This group includes two male and three female mice.

Interestingly, the three female mice exhibited a significant weight gain and scored normal clinically till day 4 p.i. and subsequently, developed clinical signs of mild-ruffled, ruffled fur, hunched back and listlessness. The greater protection observed in the female mice appears to be due to a dose effect. The infected female mouse that received the 1.25 mg/kg BW were <22 gm, had a BW average of 17.5 gm compared to the male mice (BW average 29.2 gm). The Kaplan-Meier survival curve indicated a 40% survival for the lower dose of NELL1 at Day 6 p.i. compared to a 20% survival observed for the higher dose of NELL1 (FIG. 1B).

Analyses of percent change in body weight, Kaplan-Meir survival, and change in cytokine expression in NELL1-treated compared to control SARS-CoV-2-infected tg-mice hACE2 were initially analyzed using Prism version 8 (GraphPad). The Mixed-effects Model with Toeplitz errors was used to analyze changes in weight by groups over time. Cox proportional hazard model was used to analyze the survival data. This preliminary study of delivery of NELL1 (1.25 mg/kg and 2.5 mg/kg body weight and administered at Day 0 and 3 post infection) to SARS-CoV-2 infected tg mice hACE2r, indicates that there is a dose dependent effect. Further studies will establish the optimal dose and administration regime in tg-mice hACE2r. One study design could consist of administering NELL1 protein at doses ranging from 0.75, 1.25 and 2.0 mg/kg BW to 14 tg-mice hACE2r in each group (7 male and 7 female) per dose infected with 2800 pfu of SARS-CoV-2. According to the initial statistical analyses, a minimum of 6 samples will be sufficient to yield a power of 80%.

In summary, a 2.8×10³ pfu viral dose was delivered by intranasal injection and induced a dramatic weight loss in SARS-CoV-2 infected untreated control mice immediately at one day post infection, deteriorated health condition (ruffled fur, hunched back and reduced mobility) and the untreated infected hACE2 transgenic mice met criteria for euthanasia at 4-5 days post infection. In contrast, NELL1-treated animals at the lower dose of 1.25 mg/kg body weight administered at 0 and 3 days post infection did not manifest significant weight loss until 6 days post infection. Thus, NELL1 improved clinical scores and survival of SARS-CoV-2-infected tg-mice hACE2.

The increased expression of NELL1 in the lung tissues will be validated by immunoblot and/or IHC. The cytokine profile (e.g., IL-6, IL-8, TNF-α, IL1-β) in the lung tissues will be analyzed by qPCR and BAL fluid (by Luminex 36-plex mouse kit, ThermoFisher). Further immunopathological analyses of lung tissues by immunohistochemistry for extracellular matrix will also be performed.

If retro-orbital injection is not the most effective means of the delivery of NELL1 to the lungs, other delivery routes will be analyzed, including intranasal injection and/or tracheal injection.

The pilot experiment described above used two time points of administration (Day 0 and Day 3 post-infection). Further studies will first analyze an administration regime of alternate days post-infection. The viral load, clinical scores and survival will be determined as described above. Once these are optimized, the efficacy of NELL1 in HIS-DRAGA mice model may be analyzed as described in Example 2.

Similar dose escalation studies of NELL1 delivery will be performed in IAV (H1N1 and H3N2)-infected tg mice model to establish the most optimal administration regime, prior to testing the efficacy in HIS-DRAGA mice.

### Example 2

### Efficacy of NELL1 on Repair of Immunopathological Changes and Rescue from the Cytokine Storm in HIS-DRAGA Mice Infected with SARS-CoV-2 and Influenza A Viruses

The HIS-humanized mouse strain (DRAGA mouse: HLA-DR4. HLA-A2.1. IL-2Rγc KO. RAG KO. NOD (Danner et al. (2011) PLoS One 6:e19826; Majji et al. (2016) Scientific reports 6:28093) that is HIS (human immune system)-reconstituted upon infusion with hematopoietic stem cells (HSC) from HLA-matched umbilical cord blood, lacks the murine immune system while expressing a long-lived functional HIS. This mouse responds vigorously with specific T and B cell responses to infection or immunization with various pathogens including malaria protozoans, HIV, ZIKA, Scrub Typhus, and Influenza type A heterosubtypes (Wijayalath et al. (2014) Malar J 13:386; Jiang et al. (2018) Front Immunol 9:816; Kim et al. (2017) Front Immunol 8:1405; Yi et al. (2017) EBioMedicine 25:87-97; Mendoza et al. (2018) Hum Vaccin Immunother 14:345-360; Mendoza et al. (2020) Hum Vaccin Immunother 1-16; Majji et al, (2018) Malar J 17:114), In addition, this mouse has been also validated as a model of infection with several pathogens, e.g., influenza A virus (IAV) heterosubtypes in which the mice developed significant lung pathology, including severe hemorrhagic responses to infection with higher IAV doses (Mendoza et al. (2020)).

Recently, the HIS-DRAGA mouse has been established as the first HIS-humanized model for IAV heterotypic infections (Mendoza (2018); Mendoza (2020)). When IAV-infected, the mice mimic closely the human influenza pathology and develop human lung-resident CD103⁺CD8⁺ T cells, indicating they are an excellent model not only to study influenza pathology, but also for mechanistic studies of the human immune responses to IAVs. In addition, it has recently been demonstrated that IAV-infected HIS-DRAGA mice transmit the virus to uninfected, co-caged mice (data not shown).

Since it has been found that the HIS-DRAGA mice can reconstitute human lung ECs (Brumeanu et al. (2020) “A human-immune-system mouse model for COVID-19 research (DRAGA mouse: HLA-A2.HLA-DR4.Rag1KO.IL-2Rγc KO.NOD)” BioRxiv), lung and liver EDs (Wijayalath et al. (2014) Malar J 13:386), and the human ACE2 receptor for SARS-CoV-2, two pilot experiments have been carried out indicating that these mice sustain infection with SARS-CoV-2 virus and show human-like immunopathology (Brumeanu et al. (2020)).

In a first pilot experiment, 3 HIS-DRAGA mice were infected by the i.n. route with 2.8×10³ (1 male+1 female) or 2.8×10⁴ pfu (1 female) of SARS-CoV-2 virus. The male mouse infected with the low dose succumbed to infection after 24 hours, while both female mice sustained the infection, showing an abrupt loss in body weight at 1 dpi, with ruff fur, hunched back, and reduced mobility. The surviving female mouse infected with the low dose regained its weight and mobility at 10 dpi, while the female mouse infected with the high dose remained below 10% of its original weight at 14 dpi (Brumeanu et al. (2020)). The female mouse recovered from the infection showed a high titer of Spike (RBD)-specific human IgM serum antibodies (OD450ₙₘ=0.376 at 1:20 dilution). A second ongoing pilot experiment in which 15 HIS-DRAGA female and male mice were infected i.n. with a low dose (10³ pfu) of SARS-CoV-2 virus, showed that a female mouse recovered from the infection 7 days post-infection, whereas some are still below their original body mass at 14 days post-infection (Brumeanu et al. (2020)).

As described in recent reports of human autopsies (Sauter et al. (2020) “Insights into pathogenesis of fatal COVID-19 pneumonia from histopathology with immunohistochemical and viral RNA studies” Histopathology; Bradley et al. (2020) “Histopathology and ultrastructural findings of fatal COVID-19 infections in Washington State: a case series” Lancet; Zhou et al, (2020) “The pathological autopsy of coronavirus disease 2019 (COVID-2019) in China: a review” Pathog Dis 78), the H&E and Mason-trichrome staining of lung sections from SARS-CoV-2 infected HIS-DRAGA mice showed heavy parenchymal infiltrates with T cells around the large bronchioles and at peripheral lung areas and arteriolar micro thrombi throughout the lung parenchyma (Mendoza (2020)). These results demonstrate SARS-CoV-2 infected DRAGA mice can sustain long enough the infection to develop human-like lung immunopathology.

The DRAGA mouse has been established as the first model for inducible and transmissible A/H1N1 and A/H3N2 heterotypic infections (Mendoza (2018); Mendoza (2020); Majji (2018); Brumeanu (2020)). Lungs from infected DRAGA mice closely resemble the human lung pathology of influenza infection, and they respond to infection by developing neutralizing antibodies and human lung-resident CD8⁺CD103⁺ T cell clusters in the lung intra-epithelial niches (Mendoza (2020)).

High concentrations of inflammatory cytokines in the lungs leads to deleterious effects on the lung tissue. Increased levels of IFNγ and TNFα mRNA transcripts were found in the visibly-affected lung areas from H1N1/PR8-infected DRAGA mice advancing toward severe viral pneumonia (Mendoza (2020)).

It has also been reported that increased amounts of IFN-γ and TNF-α mRNA transcripts in the lungs of severely infected DRAGA mice with H1N1/PR8 or Aichi virus occurred in the context of heavy intra-alveolar infiltration with lymphocytes, lung hemorrhage, and disruption of bronchoalveolar architecture (Mendoza (2018); Mendoza (2020); Majji (2018); Brumeanu (2020)). Together, these data strongly suggest that the IAV-infected DRAGA mice closely mimics the immunopathological events described in the lungs of humans exiting from severe influenza infection. Unfortunately, at high virus loads in the lungs, the same CD8 T protective cells can also inflict deleterious damage on the lung tissue by triggering hypercytokinemia (“cytokine storm”), haemophagocytic lymphohistiocytosis, and alveolar infiltration with lymphocytes and monocytes. Such deleterious events have been noticed in early studies of humans exiting from severe IAV pneumonia.

The DRAGA mouse model of IAV or SARS-CoV-2 infections will be employed to timely-monitor the conditions under which human lung-resident CD8 T-cells inflict damage in the lung tissue. The best therapeutic regimen of NELL1 administration established in the hACE2 tg mice in Example 1 will be tested in HIS-DRAGA mice infected with SARS-CoV-2 or IAVs. Mice will be monitored by similar assessments like in the hACE2 tg mice. Immunopathological changes in the lungs and organs such as the cytokine storm and coagulopathologies reported in severely infected humans with COV-2, H1N1, and H3N2 viruses, will be investigated in the context of human immune responses in HIS-DRAGA mice. Subsequently, the efficacy of NELL1 on repairing the lung functions by measuring the airway resistance (FEV_{0.1}) will be determined.

The A/H1N1/PR8 and A/H3N2/Aichi viruses are inoculated in DRAGA female and male mice by the intranasal (i.n.) route. The mice will only be lethally infected with a previously established LD₁₀₀ dose (100,000 EICD₅₀) of A/H1N1/PR8/34 or A/H3N2/Aichi/68. Unscheduled euthanasia will be carried out whenever the mice display a grade 4 clinical score and/or 35% loss in body mass. Clinical scoring of infection will be recorded by a qualified IACUC person blinded to the study design, as follows: 0=no visible signs of disease; 1=slight ruffled fur; 2=ruffled fur and reduced mobility; 3=ruffled fur, reduced mobility, and rapid breathing; 4=ruffled fur, minimal mobility, huddled appearance, and rapid breathing; 5=lethargy and imminent death.

Virus load in the lungs and bronchoalveolar lavage fluid (BALF) from individual infected mice will be measured based on RT-qPCR CT values using primers designed for PR8/HA and Aichi/HA (Mendoza (2018); Mendoza (2020); Majji (2018); Brumeanu (2020)).

Harvesting T cells by bronchoalveolar lavage (BAL) from infected DRAGA mice is carried out by 2 consecutive intra-tracheal (i-t) infusions/aspirations of 0.5 ml RPMI using a 22G indwelling cannula, shortly after euthanasia, cells are pelleted by centrifugation, and subjected to several analyses as described.

Pairwise curves comparison of the clinical scores between genders, and between control groups and infected and/or cross-infected mice will be analyzed by Gehan-Breslow-Wilcoxon test with Bonferroni's corrected threshold of significance. Significant differences between genders and infected versus control groups in the clonal size, phenotypes, and transcriptomics of human lung-resident CD8 T cells at 3, 9, and 30 days post-infection and re-infection will be determined by the analysis of variance using the GraphPad Prism software v5.0.

Four to five month-old, fully HIS-reconstituted DRAGA female (n=30) and male mice (n=30) will be infected i.n. with high and low CoV-2 doses (strain USA-WA1/2020, BEI Resources NR-52281, batch #70033175) at a BSL3 facility, as previously carried out in the two pilot experiments (2.8×10⁴ and 10³ pfu). These virus doses are expected to induce severe infection, and result in long-term sustainable infection from which some mice will self-cure.

The rationale of using DRAGA female and male groups is to determine whether gender differences in susceptibility to the infection occur, and whether gender differences occur in terms of severity of immunopathology in various organs. Previous studies of upper respiratory infections, including influenza in humans, wild-type mice (Robinson et al. (2014) J Virol 88:4711-4720; Vom Steeg and Klein (2019) Semin Immunopathol 41:189-194; Furman et al. (2014) Proc Natl Acad Sci USA 111:869-874; Klein et al. (2012) J Leukoc Biol 92:67-73), the HIS-DRAGA mice (Mendoza (2018); Mendoza (2020); Majji (2018); Brumeanu (2020)), and recently COVID19 patients, showed higher resilience of females than males to the infections. To follow accurately the possible role of estrogens in resilience to infection in female mice, clinical scores and results of the assays described below will be interpreted in the context of 17β-estradiol levels measured by ELISA in sera collected prior to the infection, at 7 and 14 days post-infection.

Euthanasia will be carried out in three mice from each group of mice at day 4 post-infection to determine the viral load in the organs (lungs, liver, kidneys, intestine, brain, and heart) by RT-qPCR using specific primers for Si protein. Unscheduled euthanasia will occur whenever the mice display a grade 4 clinical score and/or 20% loss in body mass. Clinical scoring of infection will be recorded by a qualified veterinarian which is blinded to the study design, as follows: 0=no visible signs of disease; 1=slight ruffled fur; 2=ruffled fur and reduced mobility; 3=ruffled fur, reduced mobility, and rapid breathing; 4=ruffled fur, minimal mobility, huddled appearance, and rapid breathing; 5=lethargy and imminent death.

The best therapeutic regimen of NELL1 administration established in the hACE2 tg mice in Example 1 will be tested in HIS-DRAGA mice infected with SARS-CoV-2 or IAVs. Four to five month-old, fully HIS-reconstituted DRAGA female (n=30) and male mice (n=30) will be infected i.n. with SARS-CoV-2 or A/H1N1/PR8 and A/H3N2/Aichi viruses. Clinical scoring, analyses of viral load, BAL fluid will be performed as described in Example 1.

Mortality due to upper respiratory infections including COVID-19 is mainly attributed to hyper inflammatory responses with excessive secretion of cytokines and chemokines in the lungs, as noted in severely infected patients (Coperchini et al. (2020) Cytokine Growth Factor Rev 53:25-32). Unfortunately, high virus loads in the lungs or prolonged presence of lung-resident CD8⁺ T cell cytolytic activity can lead to deleterious lung damage by triggering hypercytokinemia (“cytokine storm”), haemophagocytic lymphohistiocytosis, which in turn can exacerbate the alveolar infiltration with lymphocytes and monocytes (La Gruta et al. (2007) Immunol Cell Biol 85:85-92; Herold et al. (2015) Eur Respir J 1463-1478; Oldstone and Rosen (2014) Curr Top Microbiol Immunol 378:129-147) and trigger hemorrhagic events (Gilbert et al. (2010) Respiratory care 55:623-625). Such deleterious events have been described in patients exiting from COVID-19.

The murine and human inflammatory cytokines and chemokines in severely-infected HIS-DRAGA mice and in those with prolonged recovery time will be measured in sera using Pro-inflammatory 9-plex ELISA kits (IL-1β, IL-2, IL-6, IL-8, IL-12p70, GM-CSF, IFNγ, TNFα, TGFβ, and IL-10) and Chemokine 9-plex ELISA kits (Eotaxin, MIP-1β, Eotaxin-3, TARC, IP-10, IL-8, MCP-1 (MCAF/CCL2), MDC, and MCP-4 (Anogen, USA), following protocols that have been established to profile human samples (Karim et al. (2020)Front Immunol 11:1219). Plasma and serum samples will be collected and levels of pro-inflammatory markers will be measured in a BSL2+ facility.

Furthermore, RNA extracted from snap-frozen lungs of severely-infected HIS-DRAGA mice and from those with prolonged recovery time will be used to quantify, by RT-qPCR, the major pro-inflammatory murine and human cytokines such as IFN-γ, TNF-α, and IL-6.

The lung tissue damage in severe influenza infection or SARS-CoV-2 infection is not only the result of ECs apoptosis of type 1 and 2 alveolar cells following the virus invasion, but also occurs by excessive CD8 T cell killing of infected ECs. These events lead to high release of inflammatory cytokines and chemokines with destructive effects on the lung tissue. The number of apoptotic ECs in lungs will be timely-monitored and compared from groups of DRAGA mice that recovered and that did not recover from infection, by CLSM of lung sections double-stained for mouse CD326 (EC marker) and Annexin V (early apoptosis). These assays will be paralleled by CLSM on triple stained lung sections for human CD103, CD8, and Granzyme A to detect the cytolytic activity of hu lung-resident T cytolytic cells involved in ECs apoptosis.

Patients with severe COVID-19 show a hyper-inflammatory response to the infection, often accompanied by unusual patterns of microvascular thrombosis, elevated levels of fibrinogen, fibrin D-dimer and other acute-phase markers, and clinical sequelae that can include permanent lung damage, myocarditis, sepsis, and multiple organ failure (Varga et al. (2020) Lancet 395:1417-1418; Ackermann et al. (2020) N Engl J Med 383:120-128; Poissy et al. (2020) Circulation 142:184-186; Helms et al. (2020) Intensive Care Med 46:1502-1503; Helms et al. (2020) Intensive Care Med 46:1089-1098; Pavoni et al. (2020) J Thromb Thrombolysis 50:281-286; Ranucci et al. (2020) J Thromb Haemost 18:1747-1751; Wright et al. (2020) J Am Coll Surg 231:193-203 e191). These planned experiments will indicate if the HIS-DRAGA mouse model is appropriate to test therapeutic agents designed to prevent or mitigate immunothrombosis in the lung and other organs.

Heavily intra-alveolar infiltration in severe respiratory infections including SARS COVID 19 often leads to a fatal outcome due to catastrophic hypoxemic respiratory failure. Lungs from severely infected DRAGA mice will be first assessed for the extent and nature of intra-alveolar infiltrates and hemorrhagic events by HE staining in parallel with CLSM on lung sections stained for hCD3, mouse CD14/CD16 (monocytes), and human and mouse CD31/CD41/CD61 (platelets). It will be determined if mice with long recovery times from infection show pulmonary sequelae. Influenza A studies have indicated that lung healing post-IAV severe infections occurs by a process of building collagen-based fibrotic tissue in the damaged areas. The occurrence of pulmonary fibrotic sequelae in SARS-CoV-2 infected DRAGA mice with long recovery time will be determined by the extent of collagen deposition in the lungs, as revealed by the conventional Masson's trichrome staining, and by the amount of hydroxyproline in lung hydrolysates (μg/mg lung tissue) measured with Ehrlich reagent. IHC assays will detect and quantify fibrin and complement component deposition (e.g. via goat anti-mouse complement C3-Fab2-FITC, MP Biomedicals, unconjugated anti-CD3 for ELISA and Westerns, and rabbit anti-mouse Factor H related protein B, ThermoFisher). Coagulation-relevant markers including tissue factor (polyclonal antibody AF2339-SP, R&D Systems) and tissues will be stained for endothelial protein C receptor (EPCR), using polyclonal and monoclonal antibodies from R&D Systems. Properties of specific endothelial cells, healthy and damaged in areas adjacent to viral infection sites, will be evaluated by staining for markers including CD31, CD36, FABP5, CD54/ICAM-1, CD102/ICAM-2, CD106/VCAM-1, CD62E, CD62P, CD121/IL-1R, and VE-cadherin. These initial exploratory assays will allow the characterization of sites of endothelial and epithelial damage, assess the possible role of dysregulated complement activation, and identify the most significant markers of tissue damage.

In addition to these assays, RT-qPCR will be carried out of lung lysates to quantify murine and human factor VIII (FVIII) mRNA, as in the previous study of hemorrhagic damage to lungs of IAV-infected DRAGA mice (Mendoza et al. (2020) Hum Vaccin Immunother 1-16), using sensitive assays developed in the Pratt laboratory (Dutta et al. (2016) Blood Adv 1:231-239). FVIII is expressed primarily, and likely exclusively, in endothelial cells. Therefore, this assay may be used to assess the relative levels, and hence extent of damage, the engrafted human EDs, compared to damage of murine EDs, following infection with SARS-CoV-2, and also in response to therapeutic agents administered before or after infection.

Standard coagulation/fibrinolysis tests will be applied, including PT and APTT, adapting existing chromogenic assays for small volumes similar to protocols that have been long used in studies of hemophilia A (Karim et al. (2020) Front Immunol 11:1219; Gunasekera et al. (2015) Blood 895-904; Parvathaneni et al. (2017) Transl Res 187:44-52). ELISA-based tests of plasma and serum will be used to quantify circulating fibrinogen levels (Abcam kit ab213478), soluble fibrin D-dimer (mouse D-dimer ELISA kit LS-F6179, LSBio), thrombin-antithrombin (Abcam kit), thrombomodulin (polyclonal antibody AF3894-SP, R&D Systems), ferritin (rabbit mAb ab74973, Abcam) and fibrinopeptide A (LSBio kit) levels. Acute phase proteins CRP (rabbit anti-mouse CRP, Abcam), VWF (polyclonal antibody A008229-5, Agilent) and factor VIII levels will also be measured using established in-house ELISA assays as well as commercial kits and ADAMTS13 (Abcam ab71550), which is required for proper cleaving of ultra-large VWF multimers (Pipe et al. (2016) Blood 128:2007-2016) that have been hypothesized to play a role in at least some of the observed coagulopathies of COVID-19 patients. The VWF multimeric size distributions of infected and non-infected DRAGA mice will also be evaluated using standard methods employing agarose gels/Westerns. Troponin levels in plasma or serum will be measured (Abcam kit) as an indirect indicator of myocardial damage.

Results of all coagulation and fibrinolysis assays will be correlated with clinical scores of infected HIS-DRAGA mice. Data from the RT-qPCR on viral loads in the DRAGA organs and immune responses correlated with results of cytokines/chemokines assays shall provide meaningful correlates between the virus loads and the extent of endotheliopathies and coagulopathies.

The lung function will be analyzed by measuring the airway resistance (FEV_{0.1}) per group. The pulmonary function has been assessed in various diseases using an invasive method, Forced oscillation technique (FOT) (Devos et al. (2017) Respir Res 18:123). A concurrent measurement of forced expiratory volume at 0.1 s (FEV_{0.1}) as well as airway resistance (Rn) are used for clinical assessment of lung function. For FEV_{0.1} measurement in mice, the airways of mice are exposed to a negative pressure, which generates a forced expiratory flow signal. This technique simulates the assessment of human lung function, and measurement of FEV_{0.1} is preferred for pre-clinical testing in mice over the classical airway resistance (Rn) measurements.

Further studies include using a combination therapy, co-administration of NELL1 with approved anti-viral drugs like Remdesivir or with siRNAs against the viral genome. The selection of combination/cocktail therapies with NELL1 will be determined based on the integration of results of the assays described above. These data will indicate which components of the cytokine storm and/or aspects of lung pathologies were not sufficiently addressed by the administration of NELL1 protein alone.

### Example 3

### Effects of NELL1 on Virally-Induced Lung Tissue Injury

Human in vitro models (2-D monocultures of airway epithelium or 3-D tissue microphysiological systems) of lung tissue injury brought about by viral introduction in cell culture or addition of pro-inflammatory molecules to mimic the cytokine storm (multiple cytokines added to lung tissue) to induce cell injury and death are used to demonstrate the efficacy of NELL1 in regenerating virally-induced lung tissue injury. Addition of NELL1NV1 in varying doses are added into culture and examination of cytokine levels and cell/tissue viability, morphology, cell division and differentiation are evaluated by molecular and/or imaging methods. These experiments will show a reduction of inflammation and cell death and promotion of tissue regeneration (growth and differentiation) (Viola H et al. 2019 APL Bioeng 3, 041503 doi: 10.1063/1.5111549; Ng L F P et al. 2004 BMC Infectious Diseases 4:34 doi:10.1186/1471-2334-4-34).

Testing of efficacy is also achieved using small animal models such as mice, rats, ferrets and rabbits. Coronavirus infection (intranasal delivery) is introduced into inbred mouse strains like BALB/c, C57BL6 and 129S, resulting in pneumonitis with diffuse alveolar damage, characteristic of coronavirus infection. Transgenic mice expressing the human ACE2 receptor and adaptation of SARS-CoV to mice by serial passage are also commercially available. Coronavirus infection in these models are severe and result in lethality, just like in human patients (Gretebeck L M and Subbarao K 2015 Current Opinion in Virology 13:123-129; jax.org/news-and-insights/2020/february/introducing-mouse-model-for-corona-virus on the world wide web).

Large animal models such as rhesus macaques, cynomolgous macaques and African green monkeys (non-human primates) have been utilized for SARS coronavirus infection and testing of vaccines and therapeutics. These models mimic the inflammatory responses, pneumonitis and acute lung injury in human patients and are used to the test the effects of NELL1 on these pathologies that are virally-induced (Gretebeck L M and Subbarao K 2015 Current Opinion in Virology 13:123-129).

Additional large animal ARDS models using non-viral agents such as pro-inflammatory endotoxin from bacteria (LPS) and oleic acid or physical methods (repeated lavage, ventilation induced) that injure the lungs have also been developed and are used to test the effects of NELL1 on virally-induced ARDS (Ballard-Croft C et al. (2012) Ann Thoc Surg 93:1331-1339; Bastarache J A and Blackwell T S 2009 Dis Model Mech 2(5-6):218-223).

Several human models of lung injury are also used to address components of the disease that are not easily recapitulated in animal models. These models include: a) surgically-induced in vivo models where one lung is ventilated (OLV or one lung ventilation) to collapse the other lung, b) in vivo model of inhalation, intratracheal injection or intraperitoneal injection of lipopolysaccharides (LPS), a component of gram-negative bacteria, and c) ex vivo isolated perfused lung models (Proudfoot A G et al. (2011) Disease Models and Mechanisms 4:145-153 doi: 10.1242/dmm. 006213).

### Example 4

### Effects of NELL1 on Virally-Induced Heart Tissue Damage

A demonstration of the NELL1/NV1 efficacy for regenerating virally-induced heart injury is conducted by using and modifying existing human in vitro cardiac models (monolayer, 2D and 3D tissue systems) of acute myocardial tissue injury to accommodate the conditions of a viral infection. These experiments are performed by: a) direct infection by the viral pathogen (e.g. SARS-CoV or SARS-CoV-2), b) triggering a high level of inflammation with one or a combination of pro-inflammatory molecules (e.g. cytokines IL 1, IL6, TNF-alpha) implicated in generating and promoting the “cytokine storm”, or c) severe hypoxic conditions. Moreover, a combination of b) and c) is applied to model the simultaneous presence of both high inflammation and hypoxia during a severe viral infection like that manifested by SARS-CoV-2 and its variant strains. Heart tissue injury and recovery by application of the NELL1 therapeutic are assessed with morphological and histopathological observations, immunohistochemical or molecular analysis of cardiac damage markers (e.g. troponins like hs-TnI or TnT), or cytological techniques measuring cell death or apoptosis. More comprehensive analyses like transcriptomics, metabolomics and proteomics assessments are also used for evaluation of efficacy, mechanism of action and cardiotoxicity.

Myocardial cells from human and relevant model animals (e.g. rodents) are obtained from primary cells harvested from donor heart tissue, established cell lines or from human cardiomyocytes differentiated from induced pluripotent stem cells (hiPS-CMs) that are commercially available (Savoji H et al. 2019 Biomaterials 198:3-26; Wei H et al. 2019 Biochem Biophys Res Comm 520: 600-605; iCell Cardiomyocytes Application Protocol. Modeling Cardiac Ischemia: Hypoxia Induction for Cardioprotection Screening. Cellular Dynamics International). There are also available 2D and 3D cardiac models that are fabricated and used for assessing therapeutic efficacy, mechanisms of action, and cardiotoxicity of candidate molecules (Savoji H et. al. 2019 Biomaterials 198:3-26). 2D cardiac tissues are manufactured with aligned cardiomyocytes to create native-like cardiac monolayers that exhibit the conductive properties of normal heart tissue. Highly complex 3D in vitro models are also used to recapitulate the physiological and anatomical structure of the native heart-encapsulating cells in hydrogels, seeding cells into fabricated structures, decellularized extracellular matrix (ECM) of heart tissue and overlaying layers of 2D cell sheets.

NELL1/NV1 in varying doses are added into cultured cells (monolayers, 2D or 3D systems) and cardiac injury biomarker levels are measured to assess the therapeutic effects and the appropriate dosing. A dose range of 50 ng/mL-1000 ng/mL was therapeutically effective in a variety of non-heart cell lines such as skeletal fibroblasts and myoblasts under wound healing or muscle atrophy assays. Since NELL1 effects on cardiomyocytes under cytokine storm conditions or a viral infection are a new environment for NELL1NV1 activity, a much wider dose range of 10-2000 ng/mL is used for initial testing.

In vivo models using small (e.g. rats, mice, rabbits and cats) and large animals (e.g. dogs, sheep and pigs) are also routinely used for cardiovascular applications. Surgical, chemical, and genetic modifications are used to generate appropriate models for cardiovascular injury and disease (Savoji H et al. 2019 Biomaterials 198:3-26). The k18-hACE2 transgenic mouse strain was developed by Dr. Paul McCray from the University of Iowa, in collaboration with the Jackson Laboratory and is an excellent small animal in vivo experimental platform for COVID-19 research (jax.org/news-and-insights/2020/february/introducing-mouse-model-for-corona-virus on the world wide web). This mouse line has been engineered to carry the human ACE2 receptor for more efficient infection by SARs-CoV and SARS-CoV-2 via intranasal inoculation. The severity of the infection mirrors the rapid severity and lethality observed in human hosts. Symptoms (weight loss, lethargy and respiratory distress) are apparent in 3 days and the mice die within a week (7 days). NELL1NV1 recombinant protein is injected intraperitoneally, subcutaneously, intramuscularly or via tail vein injection (intravenous) at varying doses to infected mice daily at T0 to T7 (Days 1-7). NV1 or NELL1 native full-length form are administered in doses at 1×, 2×, 4× and 10× the systemic dose used for NELL1 treatment of osteoarthritis, an inflammatory condition that affects a soft tissue (cartilage) throughout the body (Li C et al. 2020 Biomaterials 226:119541). Efficacy of the treatment is evaluated by measuring mortality rates, weight, behavioral observations and multiplex cytokine assays from blood, and histopathology of lung, heart, kidney and gastrointestinal tissues.

### Example 5

### Development of Aerosol-Based NELL1 for Delivery Via Nebulizer to NHP Models

Efficient nebulizers have the potential to improve delivery of treatments to the inflamed and injured lung. Newer high-performance aerosol delivery such as vibrating mesh nebulizers (VMNs) have the potential to rapidly deliver therapeutic proteins to the distal airspaces, or during mechanical ventilation of COVID-19 patients or Influenza patients with severe pneumonia.

In these studies, the aerosol and delivered dose of NELL1 will be optimized and characterized using bench simulation and subsequently in NHP models. These data will both guide the pre-clinical study design, and de-risk the pre-clinical drug development program prior to testing in humans. Formulation optimization of NELL1 for aerosol-mediated delivery will be performed in order to ensure compatibility with the vibrating mesh nebulizer. Screening of combinations of vibrating mesh type and formulation design will lead to development of an optimal formulation for reliable and reproducible dosing.

Following identification of optimal formulation and mesh combination, the aerosol characteristics shall be assessed in order to ensure that a respirable aerosol is being produced. In line with international regulatory standards and requirements (e.g. FDA, IND, CE Mark, ISO), methods such as laser diffraction and cascade impaction will be used to assess the respirable fraction.

Aerosol-mediated delivery will be characterized using standardized protocols, in a bench model of simulated mechanical ventilation and spontaneous breathing humans. Further, we will assess the potential delivered dose in a model of NHP, in line with publications using similar models (MacLoughlin et al. (2016) J Aerosol Med Pulm Drug Deliv 29:281-287).

Further studies will advance to the NHP model with a strong prediction of the delivered dose. As indicated in Table 2, cynomolgous macaques will receive NELL1 (Isotope or label) via nebulizer of single or multiple doses. The breathing pattern will be analyzed by measuring tidal volume (Vt), breaths per minute (BPM) and ratio of inspiratory to expiratory time (I:E ratio) as described in MacLoughlin et al.

TABLE 2
Biodistribution analyses. Delivery of NELL1 by nebulizer and
analyses in lung tissues of NHP (no viral challenge).

| N | | | |
|---|---|---|---|
| (M/F) | Route | Intervention | Necropsy |
| 10 | Aerosol Spray/ | Days 1, 3, 6, 13 and 20 | 2, 4, 7, 14, 21 |
| | Nebulizer | | (N = 2 per time point) |

A preliminary study using ProteinX indicates that the fine particles generated by VMNs were a substantial fraction of the total (data not shown). Similar dosing will be analyzed for NELL1. The proposed experiments are designed so as to identify the best combination of ventilator setting, nebulizer position and/or nebulizer characteristics for both the pre-clinical NHP work, and also the final intended patient population. These data will facilitate calculation of the minimum effective dose-optimized dosing regimen (e.g. a single dose vs multiple doses).

Those skilled in the art will further appreciate that the present invention may be embodied in other specific forms without departing from the spirit or central attributes thereof. In that the foregoing description of the present invention discloses exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1. A method of treating tissue damage resulting from a viral infection in a subject in need thereof, said method comprising administering an effective amount of a NELL1 polypeptide, or a nucleic acid molecule encoding the same.

2. The method of claim 1, wherein said infection is by a respiratory virus.

3. The method of claim 1 or 2, wherein said infection is by an enveloped virus.

4. The method of claim 3, wherein said enveloped virus is a coronavirus.

5. The method of claim 4, wherein said coronavirus attaches to angiotensin-converting enzyme 2 (ACE2).

6. The method of claim 4 or 5, wherein said coronavirus is severe acute respiratory syndrome coronavirus 2 (SARS-CoV-2).

7. The method of any one of claims 1-6, wherein said subject is exhibiting a cytokine storm.

8. The method of claim 7, wherein said subject has elevated levels of any one of interleukin-6 (IL-6), interferon gamma induced protein 10 (IP-10), monocyte chemotactic protein-3 (MCP-3), interleukin-1ra (IL-1ra), interferon-gamma (IFN-γ), interleukin-2ra (IL-2ra), interleukin-10 (IL-10), interleukin-18 (IL-18), hepatocyte growth factor (HGF), macrophage inflammatory protein 1 alpha (MIG-1a), macrophage colony stimulating factor (M-CSF), granulocyte colony-stimulating factor (G-CSF), and cutaneous T-cell-attracting chemokine (CTACK), when compared to a healthy control subject.

9. The method of claim 8, wherein said subject has blood levels of interleukin-6 (IL-6) of at least about 80 pg/ml.

10. The method of claim 6, wherein said subject is administered said NELL1 polypeptide, or a nucleic acid molecule encoding the same after testing positive for coronavirus disease 2019 (COVID-19) or when exhibiting symptoms of COVID-19.

11. The method of any one of claims 1-10, wherein said subject has pneumonia.

12. The method of any one of claims 1-10, wherein said subject has acute lung injury (ALI) or acute respiratory distress syndrome (ARDS).

13. The method of any one of claims 1-12, wherein said subject is on supplementary oxygen.

14. The method of any one of claims 1-12, wherein said subject is on artificial ventilation.

15. The method of any one of claims 1-14, wherein said tissue damage is damage to a lung tissue.

16. The method of claim 15, wherein said NELL1 polypeptide, or a nucleic acid molecule encoding the same is administered via inhalation.

17. The method of any one of claims 1-14, wherein said tissue damage is damage to a heart tissue or vasculature.

18. The method of claim 17, wherein said NELL1 polypeptide, or a nucleic acid molecule encoding the same is administered via intraarterial injection.

19. The method of claim 17 or 18, wherein said subject has elevated cardiac troponin 1 (hs-cTn1) or troponin T (TnT) levels when compared to a healthy control subject.

20. The method of any one of claims 1-14, wherein said tissue damage is damage to skeletal muscle tissue.

21. The method of any one of claims 1-15, 17, 19, and 20, wherein said NELL1 polypeptide, or a nucleic acid molecule are administered systemically.

22. A method of regenerating lung tissue in a subject, said method comprising administering to a subject with damaged lung tissue an effective amount of a NELL1 polypeptide, or a nucleic acid molecule encoding the same.

23. The method of claim 22, wherein said damaged lung tissue is a result of an infection by a virus.

24. The method of claim 23, wherein said virus is a respiratory virus.

25. The method of claim 23 or 24, wherein said virus is an enveloped virus.

26. The method of claim 25, wherein said enveloped virus is a coronavirus.

27. The method of claim 26, wherein said coronavirus attaches to angiotensin-converting enzyme 2 (ACE2).

28. The method of claim 26 or 27, wherein said coronavirus is severe acute respiratory syndrome coronavirus 2 (SARS-CoV-2).

29. The method of any one of claims 22-28, wherein said damaged lung tissue is from viral pneumonia.

30. The method of any one of claims 22-28, wherein said damaged lung tissue is from acute lung injury (ALI) or acute respiratory distress syndrome (ARDS).

31. The method of any one of claims 22-30, wherein said NELL1 polypeptide, or a nucleic acid molecule encoding the same is administered via inhalation or systemically.

32. A method of treating lung inflammation in a subject, said method comprising administering to a subject in need thereof an effective amount of a NELL1 polypeptide, or a nucleic acid molecule encoding the same.

33. The method of claim 33, wherein said lung inflammation is due to an infection by a virus.

34. The method of embodiment 33, wherein said virus is a respiratory virus.

35. The method of claim 33 or 34, wherein said virus is an enveloped virus.

36. The method of claim 35, wherein said enveloped virus is a coronavirus.

37. The method of claim 36, wherein said coronavirus attaches to angiotensin-converting enzyme 2 (ACE2).

38. The method of claim 36 or 37, wherein said coronavirus is severe acute respiratory syndrome coronavirus 2 (SARS-CoV-2).

39. The method of any one of claims 32-38, wherein said subject is exhibiting a cytokine storm.

40. The method of claim 39, wherein said subject has elevated levels of any one of interleukin-6 (IL-6), interferon gamma induced protein 10 (IP-10), monocyte chemotactic protein-3 (MCP-3), interleukin-1ra (IL-1ra), interferon-gamma (IFN-γ), interleukin-2ra (IL-2ra), interleukin-10 (IL-10), interleukin-18 (IL-18), hepatocyte growth factor (HGF), macrophage inflammatory protein 1 alpha (MIG-1a), macrophage colony stimulating factor (M-CSF), granulocyte colony-stimulating factor (G-CSF), and cutaneous T-cell-attracting chemokine (CTACK), when compared to a healthy control subject.

41. The method of claim 40, wherein said subject has blood levels of interleukin-6 (IL-6) of at least about 80 pg/ml.

42. The method of any one of claims 38-41, wherein said subject is administered said NELL1 polypeptide, or a nucleic acid molecule encoding the same after testing positive for coronavirus disease 2019 (COVID-19) or when exhibiting symptoms of COVID-19.

43. The method of any one of claims 32-42, wherein said NELL1 polypeptide, or a nucleic acid molecule encoding the same is administered via inhalation or systemically.

44. The method of any one of claims 32-43, wherein said subject has pneumonia.

45. The method of any one of claims 32-43, wherein said subject has acute lung injury (ALI) or acute respiratory distress syndrome (ARDS).

46. The method of any one of claims 32-45, wherein said subject is on supplementary oxygen.

47. The method of any one of claims 32-45, wherein said subject is on artificial ventilation.

48. A method of treating weight loss or muscle atrophy due to a viral infection in a subject in need thereof, wherein said method comprises administering to said subject an effective amount of a NELL1 polypeptide, or a nucleic acid molecule encoding the same.

49. The method of claim 48, wherein said viral infection is an infection by a respiratory virus.

50. The method of claim 48 or 49, wherein said viral infection is an infection by a coronavirus.

51. The method of claim 50, wherein said coronavirus is SARS-CoV-2.

52. The method of any one of claims 1-51, wherein said NELL1 polypeptide has an amino acid sequence having at least 80% sequence identity to the amino acid sequence set forth as SEQ ID NO: 2.

53. The method of claim 52, wherein said NELL1 polypeptide is the polypeptide of SEQ ID NO: 2.

54. The method of any one of claims 1-51, wherein said NELL1 polypeptide has an amino acid sequence having at least 80% sequence identity to the amino acid sequence set forth as SEQ ID NO: 4.

55. The method of claim 54, wherein said NELL1 polypeptide is the polypeptide of SEQ ID NO: 4.

56. The method of any one of claims 1-51, wherein said NELL1 polypeptide has an amino acid sequence having at least 80% sequence identity to the amino acid sequence set forth as SEQ ID NO: 6.

57. The method of claim 56, wherein said NELL1 polypeptide is the polypeptide of SEQ ID NO: 6.

58. The method of any one of claims 1-51, wherein said NELL1 polypeptide has an amino acid sequence having at least 80% sequence identity to the amino acid sequence set forth as SEQ ID NO: 10.

59. The method of claim 58, wherein said NELL1 polypeptide is the polypeptide of SEQ ID NO: 10.

60. The method of any one of claims 1-51, wherein said NELL1 polypeptide has an amino acid sequence having at least 80% sequence identity to the amino acid sequence set forth as SEQ ID NO: 12.

61. The method of claim 60, wherein said NELL1 polypeptide is the polypeptide of SEQ ID NO: 12.

62. The method of any one of claims 1-51, wherein said NELL1 polypeptide has an amino acid sequence having at least 80% sequence identity to the amino acid sequence set forth as SEQ ID NO: 17.

63. The method of claim 62, wherein said NELL1 polypeptide is the polypeptide of SEQ ID NO: 17.

64. The method of any one of claims 1-51, wherein said NELL1 polypeptide has an amino acid sequence having at least 80% sequence identity to the amino acid sequence set forth as SEQ ID NO: 18.

65. The method of claim 64, wherein said NELL1 polypeptide is the polypeptide of SEQ ID NO: 18.

66. The method of claim 62 or 64, wherein said NELL1 polypeptide has one or more of the properties selected from the group consisting of:
a) enhanced efficacy in tissue regeneration,
b) enhanced prevention of tissue loss,
c) enhanced promotion of wound healing,
d) easier purification,
e) higher yield, and
f) less aggregate formation,
when compared to the NELL1 polypeptide's respective full-length NELL1 protein.

67. The method of claim 66, wherein said NELL1 polypeptide lacks the carboxy-terminal 179 amino acid residues of the NELL1 polypeptide's respective full-length NELL1 protein.

68. The method of any one of claims 1-67, wherein said nucleic acid molecule is comprised within an expression vector and operably linked to a promoter.

69. The method of any one of claims 1-68, wherein said subject is a mammal.

70. The method of claim 69, wherein said mammal is a human.


SEQUENCE LISTING (19 sequences)

SEQ ID NO: 1 - DNA, 3255 bp, Homo sapiens
  misc_feature: NELL1 isoform 1
  CDS (154)..(2586)
atatgcgagcgcagcacccggcgctgccgagccacctcccccgccgcccgctagcaagtttggcggctccaagccaggcgcgcctcaggatccaggctcatttgcttccacctagcttcggtgccccctgctaggcggggaccctcgagagcgatgccgatggatttgattttagttgtgtggttctgtgtgtgcactgccaggacagtggtgggctttgggatggaccctgaccttcagatggatatcgtcaccgagcttgaccttgtgaacaccacccttggagttgctcaggtgtctggaatgcacaatgccagcaaagcatttttatttcaagacatagaaagagagatccatgcagctcctcatgtgagtgagaaattaattcagctgttccggaacaagagtgaattcaccattttggccactgtacagcagaagccatccacttcaggagtgatactgtccattcgagaactggagcacagctattttgaactggagagcagtggcctgagggatgagattcggtatcactacatacacaatgggaagccaaggacagaggcacttccttaccgcatggcagatggacaatggcacaaggttgcactgtcagttagcgcctctcatctcctgctccatgtcgactgtaacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcacacttgcccaacctgcagtgatttcttaagcctggtgcaaggaataatggatttacaagagcttttggccaagatgactgcaaaactaaattatgcagagacaagacttagtcaattggaaaactgtcattgtgagaagacttgtcaagtgagtggactgctctatcgagatcaagactcttgggtagatggtgaccattgcaggaactgcacttgcaaaagtggtgccgtggaatgccgaaggatgtcctgtccccctctcaattgctccccagactccctcccagtgcacattgctggccagtgctgtaaggtctgccgaccaaaatgtatctatggaggaaaagttcttgcagaaggccagcggattttaaccaagagctgtcgggaatgccgaggtggagttttagtaaaaattacagaaatgtgtcctcctttgaactgctcagaaaaggatcacattcttcctgagaatcagtgctgccgtgtctgtagaggtcataacttttgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgatgagaatgccatctgcaccaacactgtccagggacacagctgcacctgcaaaccgggctacgtggggaacgggaccatctgcagagctttctgtgaagagggctgcagatacggtggaacgtgtgtggctcccaacaaatgtgtctgtccatctggattcacaggaagccactgcgagaaagatattgatgaatgttcagagggaatcattgagtgccacaaccattcccgctgcgttaacctgccagggtggtaccactgtgagtgcagaagcggtttccatgacgatgggacctattcactgtccggggagtcctgtattgacattgatgaatgtgccttaagaactcacacctgttggaacgattctgcctgcatcaacctggcagggggctttgactgtctctgcccctctgggccctcctgctctggtgactgtcctcatgaaggggggctgaagcacaatggccaggtgtggaccttgaaagaagacaggtgttctgtctgctcctgcaaggatggcaagatattctgccgacggacagcttgtgattgccagaatccaagtgctgacctattctgttgcccagaatgtgacaccagagtcacaagtcaatgtttagaccaaaatggtcacaagctgtatcgaagtggagacaattggacccatagctgtcagcagtgtcggtgtctggaaggagaggtagattgctggccactcacttgccccaacttgagctgtgagtatacagctatcttagaaggggaatgttgtccccgctgtgtcagtgacccctgcctagctgataacatcacctatgacatcagaaaaacttgcctggacagctatggtgtttcacggcttagtggctcagtgtggacgatggctggatctccctgcacaacctgtaaatgcaagaatggaagagtctgttgttctgtggattttgagtgtcttcaaaataattgaagtatttacagtggactcaacgcagaagaatggacgaaatgaccatccaacgtgattaaggataggaatcggtagtttggtttttttgtttgttttgtttttttaaccacagataattgccaaagtttccacctgaggacggtgtttggaggttgccttttggacctaccactttgctcattcttgctaacctagtctaggtgacctacagtgccgtgcatttaagtcaatggttgttaaaagaagtttcccgtgttgtaaatcatgtttcccttatcagatcatttgcaaatacatttaaatgatctcatggtaaatgttgatgtattttttggtttattttgtgtactaacataatagagagagactcagctccttttatttattttgttgatttatggatcaaattctaaaataaagttgcctgttgtgacttttgtcccatctactgcatacttagtgctgagatccctgtaaaatgttttgatgaaaatatgtatgtagagtccagtcgcattatacatacatttcatagtgctgaaccttcttaaatgcctactcattcagcttaaacaggctgaagccaagtatgacaaagaggggaagggccaaaaacataatcaaagaataattttaaagagaattcttgtctctcttgcaaaaaaaaaa

SEQ ID NO: 2 - protein, 810 aa, Homo sapiens
  misc_feature: NELL1 isoform 1
MPMDLILVVWFCVCTARTVVGFGMDPDLQMDIVTELDLVNTTLGVAQVSGMHNASKAFLFQDIEREIHAAPHVSEKLIQLFRNKSEFTILATVQQKPSTSGVILSIRELEHSYFELESSGLRDEIRYHYIHNGKPRTEALPYRMADGQWHKVALSVSASHLLLHVDCNRIYERVIDPPDTNLPPGINLWLGQRNQKHGLFKGIIQDGKIIFMPNGYITQCPNLNHTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECSEGIIECHNHSRCVNLPGWYHCECRSGFHDDGTYSLSGESCIDIDECALRTHTCWNDSACINLAGGFDCLCPSGPSCSGDCPHEGGLKHNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNITYDIRKTCLDSYGVSRLSGSVWTMAGSPCTTCKCKNGRVCCSVDFECLQNN

SEQ ID NO: 3 - DNA, 3114 bp, Homo sapiens
  misc_feature: NELL1 isoform 2
  CDS (154)..(2445)
atatgcgagcgcagcacccggcgctgccgagccacctcccccgccgcccgctagcaagtttggcggctccaagccaggcgcgcctcaggatccaggctcatttgcttccacctagcttcggtgccccctgctaggcggggaccctcgagagcgatgccgatggatttgattttagttgtgtggttctgtgtgtgcactgccaggacagtggtgggctttgggatggaccctgaccttcagatggatatcgtcaccgagcttgaccttgtgaacaccacccttggagttgctcaggtgtctggaatgcacaatgccagcaaagcatttttatttcaagacatagaaagagagatccatgcagctcctcatgtgagtgagaaattaattcagctgttccggaacaagagtgaattcaccattttggccactgtacagcagaagccatccacttcaggagtgatactgtccattcgagaactggagcacagctattttgaactggagagcagtggcctgagggatgagattcggtatcactacatacacaatgggaagccaaggacagaggcacttccttaccgcatggcagatggacaatggcacaaggttgcactgtcagttagcgcctctcatctcctgctccatgtcgactgtaacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcacacttgcccaacctgcagtgatttcttaagcctggtgcaaggaataatggatttacaagagcttttggccaagatgactgcaaaactaaattatgcagagacaagacttagtcaattggaaaactgtcattgtgagaagacttgtcaagtgagtggactgctctatcgagatcaagactcttgggtagatggtgaccattgcaggaactgcacttgcaaaagtggtgccgtggaatgccgaaggatgtcctgtccccctctcaattgctccccagactccctcccagtgcacattgctggccagtgctgtaaggtctgccgaccaaaatgtatctatggaggaaaagttcttgcagaaggccagcggattttaaccaagagctgtcgggaatgccgaggtggagttttagtaaaaattacagaaatgtgtcctcctttgaactgctcagaaaaggatcacattcttcctgagaatcagtgctgccgtgtctgtagaggtcataacttttgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgatgagaatgccatctgcaccaacactgtccagggacacagctgcacctgcaaaccgggctacgtggggaacgggaccatctgcagagctttctgtgaagagggctgcagatacggtggaacgtgtgtggctcccaacaaatgtgtctgtccatctggattcacaggaagccactgcgagaaagacattgatgaatgtgccttaagaactcacacctgttggaacgattctgcctgcatcaacctggcagggggctttgactgtctctgcccctctgggccctcctgctctggtgactgtcctcatgaaggggggctgaagcacaatggccaggtgtggaccttgaaagaagacaggtgttctgtctgctcctgcaaggatggcaagatattctgccgacggacagcttgtgattgccagaatccaagtgctgacctattctgttgcccagaatgtgacaccagagtcacaagtcaatgtttagaccaaaatggtcacaagctgtatcgaagtggagacaattggacccatagctgtcagcagtgtcggtgtctggaaggagaggtagattgctggccactcacttgccccaacttgagctgtgagtatacagctatcttagaaggggaatgttgtccccgctgtgtcagtgacccctgcctagctgataacatcacctatgacatcagaaaaacttgcctggacagctatggtgtttcacggcttagtggctcagtgtggacgatggctggatctccctgcacaacctgtaaatgcaagaatggaagagtctgttgttctgtggattttgagtgtcttcaaaataattgaagtatttacagtggactcaacgcagaagaatggacgaaatgaccatccaacgtgattaaggataggaatcggtagtttggtttttttgtttgttttgtttttttaaccacagataattgccaaagtttccacctgaggacggtgtttggaggttgccttttggacctaccactttgctcattcttgctaacctagtctaggtgacctacagtgccgtgcatttaagtcaatggttgttaaaagaagtttcccgtgttgtaaatcatgtttcccttatcagatcatttgcaaatacatttaaatgatctcatggtaaatgttgatgtattttttggtttattttgtgtactaacataatagagagagactcagctccttttatttattttgttgatttatggatcaaattctaaaataaagttgcctgttgtgacttttgtcccatctactgcatacttagtgctgagatccctgtaaaatgttttgatgaaaatatgtatgtagagtccagtcgcattatacatacatttcatagtgctgaaccttcttaaatgcctactcattcagcttaaacaggctgaagccaagtatgacaaagaggggaagggccaaaaacataatcaaagaataattttaaagagaattcttgtctctcttgcaaaaaaaaaa

SEQ ID NO: 4 - protein, 763 aa, Homo sapiens
  misc_feature: NELL1 isoform 2
MPMDLILVVWFCVCTARTVVGFGMDPDLQMDIVTELDLVNTTLGVAQVSGMHNASKAFLFQDIEREIHAAPHVSEKLIQLFRNKSEFTILATVQQKPSTSGVILSIRELEHSYFELESSGLRDEIRYHYIHNGKPRTEALPYRMADGQWHKVALSVSASHLLLHVDCNRIYERVIDPPDTNLPPGINLWLGQRNQKHGLFKGIIQDGKIIFMPNGYITQCPNLNHTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECALRTHTCWNDSACINLAGGFDCLCPSGPSCSGDCPHEGGLKHNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNITYDIRKTCLDSYGVSRLSGSVWTMAGSPCTTCKCKNGRVCCSVDFECLQNN

SEQ ID NO: 5 - DNA, 2791 bp, Equus caballus
  misc_feature: NELL1 isoform 1
  CDS (1)..(2376)
atgggctttgggatggaccccgaccttcaaatggatattatcaccgagctcgacctcgtgaacaccacccttggagtcactcaggtgtccggactgcacaatgccagcaaagcatttttatttcaagatgtagagagagagatccatgcagccccacacgtgagtgagaaattaattcagctgttccggaataagagtgaattcacctttttggccactgtgcagcagaagccgtcaacttcaggagtgatactgtccattcgagaactggaaaacagttattttgaactggagagcagtggcctgagagatgagattcgatatcactacacacacaaggggaagcccaggacagaggcacttccctaccggatggcggacggacggtggcacaaggtggcgctgtcagttagcgcctctcatctcctgctccacatcgactgcaacaggatttatgaacgtgtgatagacactcctgagaccaacctccccccaggaagcaatttgtggctgggtcagcgaaaccaaaagcacggcttattcaaaggaatcatccaagatggaaaaatcatcttcatgccgaatggatacataacacagtgtccgaacctgaatcgcacttgcccaacgtgcagtgatttcttaagcctggtgcaaggaatcatggatttacaagagcttctggccaagatgactgcgaaactaaattatgcagagacacgacttagtcaattggaaaactgccactgcgagaagacctgtcaagtgagtggactgctctatagagaccaggactcctgggttgatggcgatcactgtaggaactgcacgtgcaaaagcggcgctgtggaatgtcggaggatgtcttgtccccctctcaattgctccccagactccctccctgtgcacgttgccggccagtgctgtaaggtctgccgaccaaaatgtatctacggagggaaagtccttgcagaaggccagcggattttaaccaagagctgtcgggaatgccgaggtggagttttagtgaaaattacagaagcgtgccctcctttgaactgctcagacaaggatcacattctcccagagaatcagtgctgcagcgtctgcagaggtcataacttttgtgcggaaggacctaaatgtggtgaaaattcagagtgcaaaaactggaatacaaaagctacttgcgagtgcaagaatggttatatctctgtccagggggactccgcctactgtgaagatatcgatgagtgtgctgctaagatgcattactgtcgtgccaatactgtgtgtgtcaacctgcctgggttatatcggtgtgactgtgtcccgggatacattcgcgtggatgatttctcttgtacagaacatgacgaatgtggcagcgggcagcacaactgtgatgagaatgccatctgcaccaacactgtccagggacacagctgcacctgcaaaccgggctacgtggggaatgggaccagctgcagagcgttctgcgaagagggctgcagatatggcgggacatgcgtggctcctaacaaatgtgtctgtccttctggattcacaggaagccactgtgagaaagatattgatgaatgtacagagggaatcattgagtgccacaaccattcccgctgcgttaacctgccagggtggtaccactgtgagtgcagaagcggtttccatgacgatgggacctattcactgtccggggagtcctgtattgacattgatgaatgtgccttaagaactcacacctgttggaatgattctgcctgcatcaacttggcagggggcttcgactgcctgtgtccctcagggccatcctgctctggtgactgcccccacgaaggaggactgaagcgcaacgggcaggtgtggaccctgaaagaagacaggtgttctgtgtgttcctgcaaggatgggaagatattctgccgacggacagcttgtgattgccagaatccaagcgttgaccttttctgttgcccagagtgtgacaccagggtcacaagtcaatgtttagaccaaaatggacacaagctctatcgaagtggagacaattggactcacagctgtcagcagtgccggtgtctggaaggagaggtagattgctggccactcacttgccccagattgagctgtgagtacacagccatcttggaaggggagtgttgtccacgctgtgtcagcgacccctgcctggcggataacatcgtctatgacatcagagaaacttgcctggacagctatggagtttcaaggcttagtggctcagtgtggacattggctggatctccctgcacgacctgcaaatgcaagaatggaagtgtctgctgttctgtggatttggagtgtcttcataataattgaaggatttaaaatggactcatgatcgccagagaaaaatggacaaatgaccatccatgatgatgaaagaacaggagttggtgttttttttaccacagacaattaccaaagtctccgtctgaggaaggtgtttgcaggttgccttttggacctcccactctgctcattcttgctaacctagtctaggtgacctacagtgcatttcagtctatggttgttaaaagaagttttccgtgttgtaaatcacgtttcccttaccaggtcattgcaaatacatttaaatgatttcatggtaaatgttgatgtattttttgggtttattttgtgtactaacataatagagattcagctgcttttatttatttttttcttgacttttggatcaaattcaacaaataaagttgcctgttgtgatttt

SEQ ID NO: 6 - protein, 791 aa, Equus caballus
  misc_feature: NELL1 isoform 1
MGFGMDPDLQMDIITELDLVNTTLGVTQVSGLHNASKAFLFQDVEREIHAAPHVSEKLIQLFRNKSEFTFLATVQQKPSTSGVILSIRELENSYFELESSGLRDEIRYHYTHKGKPRTEALPYRMADGRWHKVALSVSASHLLLHIDCNRIYERVIDTPETNLPPGSNLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHVAGQCCKVCRPKCIYGGKVLAEGQRILTKSCRECRGGVLVKITEACPPLNCSDKDHILPENQCCSVCRGHNFCAEGPKCGENSECKNWNTKATCECKNGYISVQGDSAYCEDIDECAAKMHYCRANTVCVNLPGLYRCDCVPGYIRVDDFSCTEHDECGSGQHNCDENAICTNTVQGHSCTCKPGYVGNGTSCRAFCEEGCRYGGTCVAPNKCVCPSGFTGSHCEKDIDECTEGIIECHNHSRCVNLPGWYHCECRSGFHDDGTYSLSGESCIDIDECALRTHTCWNDSACINLAGGFDCLCPSGPSCSGDCPHEGGLKRNGQVWTLKEDRCSVCSCKDGKIFCRRTACDCQNPSVDLFCCPECDTRVTSQCLDQNGHKLYRSGDNWTHSCQQCRCLEGEVDCWPLTCPRLSCEYTAILEGECCPRCVSDPCLADNIVYDIRETCLDSYGVSRLSGSVWTLAGSPCTTCKCKNGSVCCSVDLECLHNN

SEQ ID NO: 7 - DNA, 2285 bp, Equus caballus
  misc_feature: NELL1 isoform 2
  CDS (1)..(2235)
atgggctttgggatggaccccgaccttcaaatggatattatcaccgagctcgacctcgtgaacaccacccttggagtcactcaggtgtccggactgcacaatgccagcaaagcatttttatttcaagatgtagagagagagatccatgcagccccacacgtgagtgagaaattaattcagctgttccggaataagagtgaattcacctttttggccactgtgcagcagaagccgtcaacttcaggagtgatactgtccattcgagaactggaaaacagttattttgaactggagagcagtggcctgagagatgagattcgatatcactacacacacaaggggaagcccaggacagaggcacttccctaccggatggcggacggacggtggcacaaggtggcgctgtcagttagcgcctctcatctcctgctccacatcgactgcaacaggatttatgaacgtgtgatagacactcctgagaccaacctccccccaggaagcaatttgtggctgggtcagcgaaaccaaaagcacggcttattcaaaggaatcatccaagatggaaaaatcatcttcatgccgaatggatacataacacagtgtccgaacctgaatcgcacttgcccaacgtgcagtgatttcttaagcctggtgcaaggaatcatggatttacaagagcttctggccaagatgactgcgaaactaaattatgcagagacacgacttagtcaattggaaaactgccactgcgagaagacctgtcaagtgagtggactgctctatagagaccaggactcctgggttgatggcgatcactgtaggaactgcacgtgcaaaagcggcgctgtggaatgtcggaggatgtcttgtccccctctcaattgctccccagactccctccctgtgcacgttgccggccagtgctgtaaggtctgccgaccaaaatgtatctacggagggaaagtccttgcagaaggccagcggattttaaccaagagctgtcgggaatgccgaggtggagttttagtgaaaattacagaagcgtgccctcctttgaactgctcagacaaggatcacattctcccagagaatcagtgctgcagcgtctgcagaggtcataacttttgtgcggaaggacctaaatgtggtgaaaattcagagtgcaaaaactggaatacaaaagctacttgcgagtgcaagaatggttatatctctgtccagggggactccgcctactgtgaagatatcgatgagtgtgctgctaagatgcattactgtcgtgccaatactgtgtgtgtcaacctgcctgggttatatcggtgtgactgtgtcccgggatacattcgcgtggatgatttctcttgtacagaacatgacgaatgtggcagcgggcagcacaactgtgatgagaatgccatctgcaccaacactgtccagggacacagctgcacctgcaaaccgggctacgtggggaatgggaccagctgcagagcgttctgcgaagagggctgcagatatggcgggacatgcgtggctcctaacaaatgtgtctgtccttctggattcacaggaagccactgtgagaaagacattgatgaatgtgccttaagaactcacacctgttggaatgattctgcctgcatcaacttggcagggggcttcgactgcctgtgtccctcagggccatcctgctctggtgactgcccccacgaaggaggactgaagcgcaacgggcaggtgtggaccctgaaagaagacaggtgttctgtgtgttcctgcaaggatgggaagatattctgccgacggacagcttgtgattgccagaatccaagcgttgaccttttctgttgcccagagtgtgacaccagggtcacaagtcaatgtttagaccaaaatggacacaagctctatcgaagtggagacaattggactcacagctgtcagcagtgccggtgtctggaaggagaggtagattgctggccactcacttgccccagattgagctgtgagtacacagccatcttggaaggggagtgttgtccacgctgtgtcagcgacccctgcctggcggataacatcgtctatgacatcagagaaacttgcctggacagctatggagtttcaaggcttagtggctcagtgtggacattggctggatctccctgcacgacctgcaaatgcaagaatggaagtgtctgctgttctgtggatttggagtgtcttcataataattgaaggatttaaaatggactcatgatcgccagagaaaaatggacaaatgacca

SEQ ID NO: 8 - protein, 744 aa, Equus caballus
  misc_feature: NELL1 isoform 2
MGFGMDPDLQMDIITELDLVNTTLGVTQVSGLHNASKAFLFQDVEREIHAAPHVSEKLIQLFRNKSEFTFLATVQQKPSTSGVILSIRELENSYFELESSGLRDEIRYHYTHKGKPRTEALPYRMADGRWHKVALSVSASHLLLHIDCNRIYERVIDTPETNLPPGSNLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHVAGQCCKVCRPKCIYGGKVLAEGQRILTKSCRECRGGVLVKITEACPPLNCSDKDHILPENQCCSVCRGHNFCAEGPKCGENSECKNWNTKATCECKNGYISVQGDSAYCEDIDECAAKMHYCRANTVCVNLPGLYRCDCVPGYIRVDDFSCTEHDECGSGQHNCDENAICTNTVQGHSCTCKPGYVGNGTSCRAFCEEGCRYGGTCVAPNKCVCPSGFTGSHCEKDIDECALRTHTCWNDSACINLAGGFDCLCPSGPSCSGDCPHEGGLKRNGQVWTLKEDRCSVCSCKDGKIFCRRTACDCQNPSVDLFCCPECDTRVTSQCLDQNGHKLYRSGDNWTHSCQQCRCLEGEVDCWPLTCPRLSCEYTAILEGECCPRCVSDPCLADNIVYDIRETCLDSYGVSRLSGSVWTLAGSPCTTCKCKNGSVCCSVDLECLHNN

SEQ ID NO: 9 - DNA, 2812 bp, Mus musculus
  misc_feature: NELL1
  CDS (40)..(2472)
gcgttggtgcgccctgcttggcggggggcctccggagcgatgccgatggatgtgattttagttttgtggttctgtgtgtgcaccgccaggacagtgctgggctttgggatggaccctgaccttcagatggacatcatcactgaacttgaccttgtgaacaccaccctgggcgtcactcaggtggctggactacacaatgccagtaaggcatttctgtttcaagatgtacagagagagatccactcagcccctcatgtgagtgagaagctgatccagctattccggaataagagtgagtttacctttttggctacagtgcagcagaagccgtccacctcaggggtgatactgtcgatccgggagctggaacacagctattttgaactggagagcagtggcccaagagaagagatacgctatcattacatccatggcggcaagcccaggactgaggcccttccctaccgcatggccgatggacagtggcacaaggtcgcgctgtctgtgagcgcctctcacctcctactccatgtcgactgcaataggatttatgagcgtgtgatagatcctccggagaccaaccttcctccaggaagcaatctatggcttgggcaacgtaatcaaaagcatggctttttcaaaggaatcatccaagatggcaagatcatcttcatgccgaacggcttcatcacacagtgccccaacctaaatcgcacttgcccaacatgcagtgatttcctgagcctggttcaaggaataatggatttgcaagagcttttggccaagatgactgcaaaactgaattatgcagagacgagacttggtcaactggaaaattgccactgtgagaagacctgccaagtgagtgggctgctctacagggaccaagactcctgggtagatggtgacaactgcaggaactgcacatgcaaaagtggtgctgtggagtgccgaaggatgtcctgtcccccactcaactgttccccagactcacttcctgtgcatatttctggccaatgttgtaaagtttgcagaccaaaatgtatctatggaggaaaagttcttgctgagggccagcggattttaaccaagacctgccgggaatgtcgaggtggagtcttggtaaaaatcacagaagcttgccctcctttgaactgctcagagaaggatcatattcttccggagaaccagtgctgcagggtctgccgaggtcataacttctgtgcagaagcacctaagtgtggagaaaactcggaatgcaaaaattggaatacaaaagcgacttgtgagtgcaagaatggatacatctctgtccagggcaactctgcatactgtgaagatatcgatgagtgtgcagcaaagatgcactactgtcatgccaacacggtgtgtgtcaacttgccggggttatatcgctgtgactgcatcccaggatacatccgtgtggatgacttctcttgtacggagcatgatgattgtggcagcggacaacacaactgtgacaaaaatgccatctgtaccaacacagtccagggacacagctgtacctgccagccaggctacgtgggaaatggtactgtctgcaaagcattctgtgaagagggttgcagatacggaggtacctgtgtggcccctaacaaatgtgtctgtccttctggattcacaggaagccactgtgagaaagatattgatgaatgtgcagagggattcgttgagtgccacaaccactcccgctgcgttaaccttccagggtggtaccactgtgagtgcagaagcggtttccatgacgatgggacctattcactgtccggggagtcctgcattgatattgatgaatgtgccttaagaactcacacttgttggaatgactctgcctgcatcaacttagcaggaggatttgactgcctgtgtccctctgggccctcctgctctggtgactgtccccacgaaggggggctgaagcataatgggcaggtgtggattctgagagaagacaggtgttcagtctgttcctgtaaggatgggaagatattctgccggcggacagcttgtgattgccagaatccaaatgttgaccttttctgctgcccagagtgtgacaccagggtcactagccaatgtttagatcaaagcggacagaagctctatcgaagtggagacaactggacccacagctgccagcagtgccgatgtctggaaggagaggcagactgctggcctctagcttgccctagtttgagctgtgaatacacagccatctttgaaggagagtgttgtccccgctgtgtcagtgacccctgcctggctgataatattgcctatgacatcagaaaaacttgcctggacagctctggtatttcgaggctgagcggcgcagtgtggacaatggctggatctccctgtacaacctgtcaatgcaagaatgggagagtctgctgctctgtggatctggtgtgtcttgagaataactgaagattttaaatggactcatcacatgagaaaatggacaaaatgaccatccaacctgaggaagaggaggggctgatttctttttctttttaaccacagtcaattaccaaagtctccatcagaggaaggcgtttgggttgcctttaccactttgctcatccttgctgacctagtctagatgcctgcagtaccgtgtatttcggtcgatggttgttgagtctccgtgctgtaaatcacatttcccttgtcagatcatttacagatacatttaaaggattccatgataaatgttaaagtaccttttgtttattttgtgtaccaacataatagagacttggcacca

SEQ ID NO: 10 - protein, 810 aa, Mus musculus
  misc_feature: NELL1
MPMDVILVLWFCVCTARTVLGFGMDPDLQMDIITELDLVNTTLGVTQVAGLHNASKAFLFQDVQREIHSAPHVSEKLIQLFRNKSEFTFLATVQQKPSTSGVILSIRELEHSYFELESSGPREEIRYHYIHGGKPRTEALPYRMADGQWHKVALSVSASHLLLHVDCNRIYERVIDPPETNLPPGSNLWLGQRNQKHGFFKGIIQDGKIIFMPNGFITQCPNLNRTCPTCSDFLSLVQGIMDLQELLAKMTAKLNYAETRLGQLENCHCEKTCQVSGLLYRDQDSWVDGDNCRNCTCKSGAVECRRMSCPPLNCSPDSLPVHISGQCCKVCRPKCIYGGKVLAEGQRILTKTCRECRGGVLVKITEACPPLNCSEKDHILPENQCCRVCRGHNFCAEAPKCGENSECKNWNTKATCECKNGYISVQGNSAYCEDIDECAAKMHYCHANTVCVNLPGLYRCDCIPGYIRVDDFSCTEHDDCGSGQHNCDKNAICTNTVQGHSCTCQPGYVGNGTVCKAFCEEGCRYGGTCVAPNKCVCPSGFTGSHCEKDIDECAEGFVECHNHSRCVNLPGWYHCECRSGFHDDGTYSLSGESCIDIDECALRTHTCWNDSACINLAGGFDCLCPSGPSCSGDCPHEGGLKHNGQVWILREDRCSVCSCKDGKIFCRRTACDCQNPNVDLFCCPECDTRVTSQCLDQSGQKLYRSGDNWTHSCQQCRCLEGEADCWPLACPSLSCEYTAIFEGECCPRCVSDPCLADNIAYDIRKTCLDSSGISRLSGAVWTMAGSPCTTCQCKNGRVCCSVDLVCLENN

SEQ ID NO: 11 - DNA, 2915 bp, Rattus norvegicus
  misc_feature: NELL1
  CDS (59)..(2491)
aagcactggtttcttgttagcgttggtgcgccctgcttggcgggggttctccggagcgatgccgatggatgtgattttagttttgtggttctgtgtatgcaccgccaggacagtgttgggctttgggatggaccctgaccttcagctggacatcatctcagagctcgacctggtgaacaccaccctgggagtcacgcaggtggctggactgcacaacgccagtaaagcatttctatttcaagatgtacagagagagatccattcggcccctcacgtgagtgagaagctgatccagctattccggaataagagcgagttcacctttttggctacagtgcagcagaaaccatccacctcaggggtgatactgtccatccgggagctggagcacagctattttgaactggagagcagtggcccaagagaagagatacgctaccattacatacatggtggaaagcccaggactgaggcccttccctaccgcatggcagacggacaatggcacaaggtcgcgctgtcagtgagcgcctctcacctcctgctccacatcgactgcaataggatttacgagcgtgtgatagaccctccggagaccaaccttcctccaggaagcaatctgtggcttgggcaacgtaaccaaaagcatggctttttcaaaggaatcatccaagatggtaagatcatcttcatgccgaatggtttcatcacacagtgtcccaacctcaatcgcacttgcccaacatgcagtgacttcctgagcctggttcaaggaataatggatttgcaagagcttttggccaagatgactgcaaaactgaattatgcagagacgagacttggtcaactggaaaattgccactgtgagaagacctgccaagtgagtgggctgctctacagggaccaagactcctgggtggatggtgacaactgtgggaactgcacgtgcaaaagtggtgccgtggagtgccgcaggatgtcctgtcccccgctcaactgttccccggactcacttcctgtgcacatttccggccagtgttgtaaagtttgcagaccaaaatgtatctatggaggaaaagttcttgctgagggccagcggattttaaccaagacctgccgggaatgtcgaggtggagtcttggtaaaaatcacagaagcttgccctcctttgaactgctcagcaaaggatcatattcttccagagaatcagtgctgcagggtctgcccaggtcataacttctgtgcagaagcacctaagtgcggagaaaactcggaatgcaaaaattggaatacaaaagcaacctgtgagtgcaagaatggatacatctctgtccagggcaactctgcatactgtgaagatattgatgagtgtgcagctaaaatgcactattgtcatgccaacaccgtgtgtgtcaacttgccggggttgtatcgctgtgactgcgtcccagggtacatccgtgtggatgacttctcttgtacggagcatgatgattgtggcagcggacaacacaactgcgacaaaaatgccatctgtaccaacacagtccagggacacagctgcacctgccagccgggttacgtgggaaatggcaccatctgcaaagcattctgtgaagagggttgcagatacggaggtacctgtgtggctcctaacaagtgtgtctgtccttctggattcacgggaagccactgtgagaaagatattgatgaatgcgcagagggattcgttgaatgccacaactactcccgctgtgttaacctgccagggtggtaccactgtgagtgcagaagcggtttccatgacgatgggacctactcactgtccggggagtcctgcattgatatcgatgaatgtgccttaagaactcacacttgttggaatgactctgcctgcatcaacttagcaggaggatttgactgcctgtgtccctctgggccctcctgctctggtgactgtccccacgaaggagggctgaagcataatgggcaggtgtggattctgagagaagacaggtgttcagtctgttcctgcaaggatgggaagatattctgccggcggacagcttgtgattgccagaatccaaatgttgaccttttttgctgcccagagtgcgataccagggtcaccagccaatgtttagatcaaagtggacagaagctctatcgaagtggagacaactggacccacagctgccagcagtgccgatgtctggaaggagaggcagactgctggcctctggcttgccctagtttgggctgtgaatacacagccatgtttgaaggggagtgttgtccccgatgtgtcagtgacccctgcctggctggtaatattgcctatgacatcagaaaaacttgcctggacagctttggtgtttcgaggctgagcggagccgtgtggacaatggctggatctccttgtacaacctgcaaatgcaagaatgggagagtctgctgctctgtggatctggagtgtattgagaataactgaagattttaaatggactcgtcacgtgagaaaatgggcaaaatgatcatcccacctgaggaagaagaggggctgatttctttttctttttaaccacagtcaattaccaaagtctccatctgaggaaggcgtttggattgcctttgccactttgctcatccttgctgacctagtctagatgcctgcagtaccgtgcatttcggtcgatggttgttgagtctcagtgttgtaaatcgcatttccctcgtcagatcatttacagatacatttaaaggggttccatgataaatgttaatgtaacttttgtttattttgtgtactgacataatagagacttggcaccatttatttatttttcttgatttttggatcaaattctaaaaataaagttgcctgttgcgaaaaaaaaaaaaaaaaaaaaaaaaaa

SEQ ID NO: 12 - protein, 810 aa, Rattus norvegicus
  misc_feature: NELL1
MPMDVILVLWFCVCTARTVLGFGMDPDLQLDIISELDLVNTTLGVTQVAGLHNASKAFLFQDVQREIHSAPHVSEKLIQLFRNKSEFTFLATVQQKPSTSGVILSIRELEHSYFELESSGPREEIRYHYIHGGKPRTEALPYRMADGQWHKVALSVSASHLLLHIDCNRIYERVIDPPETNLPPGSNLWLGQRNQKHGFFKGIIQDGKIIFMPNGFITQCPNLNRTCPTCSDFLSLVQGIMDLQELLAKMTAKLNYAETRLGQLENCHCEKTCQVSGLLYRDQDSWVDGDNCGNCTCKSGAVECRRMSCPPLNCSPDSLPVHISGQCCKVCRPKCIYGGKVLAEGQRILTKTCRECRGGVLVKITEACPPLNCSAKDHILPENQCCRVCPGHNFCAEAPKCGENSECKNWNTKATCECKNGYISVQGNSAYCEDIDECAAKMHYCHANTVCVNLPGLYRCDCVPGYIRVDDFSCTEHDDCGSGQHNCDKNAICTNTVQGHSCTCQPGYVGNGTICKAFCEEGCRYGGTCVAPNKCVCPSGFTGSHCEKDIDECAEGFVECHNYSRCVNLPGWYHCECRSGFHDDGTYSLSGESCIDIDECALRTHTCWNDSACINLAGGFDCLCPSGPSCSGDCPHEGGLKHNGQVWILREDRCSVCSCKDGKIFCRRTACDCQNPNVDLFCCPECDTRVTSQCLDQSGQKLYRSGDNWTHSCQQCRCLEGEADCWPLACPSLGCEYTAMFEGECCPRCVSDPCLAGNIAYDIRKTCLDSFGVSRLSGAVWTMAGSPCTTCKCKNGRVCCSVDLECIENN

SEQ ID NO: 13 - protein, 810 aa, Felis catus
  MISC_FEATURE: NELL1 isoform 1
MPRDVILVVWFCVCTARTVVGFGTDPDLQVDIIAELDLVNTTAGVTQVSGLHNASKAYLFQETEREIHAAPHVSEKLIQLFRNKSEFSFLATVQQKPSTSGVILSIRELEHSYFELESSGLRDEIRYHYIHNGKPRTEALPYRMADGQWHKVALSISASHLLLHVDCNRIYERVIDPPETNLPPGSNVWLGQRNQKHGLFKGIIQDGKIIFMPNGYITQCPNLNRTCPTCSDFLSLVQGIMDLQELLAKMTAKLNYAETRLNQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITDACPPLNCSEKDHILPENQCCSVCRGHNFCAEGPTCGENSECKNWNTKATCECKNGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCQEGCRYGGTCVSPNKCVCPSGFTGSHCEKDIDECTEGIIECHNHSRCVNLPGWYHCECRSGFHDDGTYSLSGESCIDIDECALRTHTCWNDSACINLAGGFDCLCPSGPSCSGDCPHEGGLKRNGQVWTLKEDRCSVCSCKDGKIFCRRTACDCQNPSVDLFCCPECDTRVTSQCLDQNGHKLYRSGDNWTHSCQQCRCLEGEVDCWPLTCPNLSCEYTAMLEGECCPRCVSDPCLADNIAYDIRKTCLDSYGISRLSGAVWTMAGSPCTTCKCKNGSVCCSVDLECLHNN

SEQ ID NO: 14 - protein, 763 aa, Felis catus
  MISC_FEATURE: NELL1 isoform 2
MPRDVILVVWFCVCTARTVVGFGTDPDLQVDIIAELDLVNTTAGVTQVSGLHNASKAYLFQETEREIHAAPHVSEKLIQLFRNKSEFSFLATVQQKPSTSGVILSIRELEHSYFELESSGLRDEIRYHYIHNGKPRTEALPYRMADGQWHKVALSISASHLLLHVDCNRIYERVIDPPETNLPPGSNVWLGQRNQKHGLFKGIIQDGKIIFMPNGYITQCPNLNRTCPTCSDFLSLVQGIMDLQELLAKMTAKLNYAETRLNQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITDACPPLNCSEKDHILPENQCCSVCRGHNFCAEGPTCGENSECKNWNTKATCECKNGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCQEGCRYGGTCVSPNKCVCPSGFTGSHCEKDIDECALRTHTCWNDSACINLAGGFDCLCPSGPSCSGDCPHEGGLKRNGQVWTLKEDRCSVCSCKDGKIFCRRTACDCQNPSVDLFCCPECDTRVTSQCLDQNGHKLYRSGDNWTHSCQQCRCLEGEVDCWPLTCPNLSCEYTAMLEGECCPRCVSDPCLADNIAYDIRKTCLDSYGISRLSGAVWTMAGSPCTTCKCKNGSVCCSVDLECLHNN

SEQ ID NO: 15 - protein, 901 aa, Canis lupis familiaris
  MISC_FEATURE: NELL1
MTSTSFLLWLGCVHNTKFPFPLVLVTRAIVVVVVEVVGVGSPGVRIRSTGCDILLLYEVLEHLLGIRFLCVDQGENSCHHGQCACRLQVIVPKALMSVFEAKTAVCFFPVVGFGTDPDLQMDIITELDLVNISLGVTQVSGLHNASKAYVFQDTAREIHAAPHVSEKLIQLFRNKSDFTFLATVQQKPSTSGVILSIRELEHSYFELESSGLRDEIRYHYMHNGKPRTEALPYRLADGQWHKVALSVSASHLLLHIDCNRIYERVIDPPETNLPPGSNLWLGQRNQKHGFFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKGGAVECRRMSCPPLNCSPDSLPVHIAGQCCKVCRPKCIYGGRVLAEGQRILTKSCRECRGGVLVKITDACPPLNCSEKDHILPENQCCSVCRGHNFCAEGPKCGENSECKNWNTKATCECKNGYISVQGDSAYCEDIDECAAKMHYCHANTVCVNLPGLYRCDCVPGYIRVDDFSCTEHDECGSGQHNCDENAICTNTVRGHSCTCKPGYVGNGTICRAFCQEGCRYGGSCVSPNKCVCPSGFTGSHCEKDIDECTEGIIECHNHSRCVNLPGWYHCECRSGFHDDGTYSLSGESCIDIDECALRTHTCWNDSACINLAGGFDCLCPSGPSCSGDCPHEGGLKRNGQVWTLKEDRCSVCSCKDGKILCRRTACDCQNPSVDLFCCPECDTRVTSQCLDQNGHKLYRSGDNWTHSCQQCRCLEGEVDCWPLTCPNLSCEYTAILEGECCPRCVSDPCLADNIAYDIRKTCLDSYGISRLSGSVWTMAGSPCTTCKCKNGSVCCSVDLECLHNN

SEQ ID NO: 16 - protein, 810 aa, Ovis aries
  MISC_FEATURE: NELL1
MPRGVILVVCFCVCAARTVVGFGMDPDLQLDIITELDLVNTTLGVTQVSGLHNTSKAFLFQDAEREIHAAPHVSEKLIQLFRNKSEFTFLATVQQKPSTSGVILSIRELEHSYFELESSGLRDEIRYHYMHSGRPRTEALPYRLADGQWHRVALSVSASHLLLHIDCNRIYERVIDPPETNLPPGSNLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILSKNCQECRGGVLVKITEACPLLNCSEKDHILPENQCCSVCRGHNFCAEGPKCGENSECKNWNTKATCECKNGYISVQGDSAYCEDIDECAAKMHYCHANTVCVNLPGLYRCDCVPGYIRVDDFSCTEHDDCGSGQHNCDENAICTNTVQGHSCTCKPGYVGNGTICRAFCEEGCRYGGTCMAPNKCVCPSGFTGSHCEKDIDECAEGIIECHSHSRCVNLPGWYHCECRSGFHDDGTYSLSGESCVDIDECALRTHTCWNDSACINLAGGFDCLCPSGPSCSGDCPHEGGLKRNGQVWTLKEDRCSVCSCKDGKIFCRRTACDCQNPSVDLFCCPECDTRVTSQCLDQNGNKLYRSGDNWTHSCQQCRCLEGEVDCWPLTCPSLSCEYTTILEGECCPRCVSDPCLADNIAYDIRKTCLDSYGLSRLSGSVWTMAGSPCTTCKCKNGSVCCSVDLECLHNN

SEQ ID NO: 17 - protein, 610 aa, Homo sapiens
  MISC_FEATURE: NELL1 fragment
FGMDPDLQMDIVTELDLVNTTLGVAQVSGMHNASKAFLFQDIEREIHAAPHVSEKLIQLFRNKSEFTILATVQQKPSTSGVILSIRELEHSYFELESSGLRDEIRYHYIHNGKPRTEALPYRMADGQWHKVALSVSASHLLLHVDCNRIYERVIDPPDTNLPPGINLWLGQRNQKHGLFKGIIQDGKIIFMPNGYITQCPNLNHTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECSEGIIECHNHSRCVNLPGWYHCECRSGFHDDGTYSLSGESCIDIDECALRTHTCWNDSACINLAGGFDCLCPSGPSCS

SEQ ID NO: 18 - protein, 610 aa, Equus caballus
  MISC_FEATURE: NELL1 fragment
FGMDPDLQMDIITELDLVNTTLGVTQVSGLHNASKAFLFQDVEREIHAAPHVSEKLIQLFRNKSEFTFLATVQQKPSTSGVILSIRELENSYFELESSGLRDEIRYHYTHKGKPRTEALPYRMADGRWHKVALSVSASHLLLHIDCNRIYERVIDTPETNLPPGSNLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHVAGQCCKVCRPKCIYGGKVLAEGQRILTKSCRECRGGVLVKITEACPPLNCSDKDHILPENQCCSVCRGHNFCAEGPKCGENSECKNWNTKATCECKNGYISVQGDSAYCEDIDECAAKMHYCRANTVCVNLPGLYRCDCVPGYIRVDDFSCTEHDECGSGQHNCDENAICTNTVQGHSCTCKPGYVGNGTSCRAFCEEGCRYGGTCVAPNKCVCPSGFTGSHCEKDIDECTEGIIECHNHSRCVNLPGWYHCECRSGFHDDGTYSLSGESCIDIDECALRTHTCWNDSACINLAGGFDCLCPSGPSCS

SEQ ID NO: 19 - protein, 599 aa, Bos taurus
  MISC_FEATURE: NELL1
MALCSFSVVGFGLDPDLQLDIITELDLVNTTLGVTQVSGLHNTSKAFLFQDAEREIHAAPHVSEKLIQLFRNKSEFTFLATVQQKPSTSGVILSIRELEHSYFELESSGLRDEIRYHYVHSGRPRTEALPYRLADGQWHRVALSVSASHLLLHIDCNRIYERVIDPPETNLPPGSNLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGECCKVCRPKCIYGGKVLAEGQRILSKSCQECRGGVLVKITEACPLLNCSEKDHILPENQCCSVCRGHNFCAEGPKCGENSECKNWNTKATCECKNGYISVQGDSAYCEDIDECAAKMHYCHANTVCVNLPGLYRCDCVPGYIRVDDFSCTEHDDCGSGQHNCDENAICTNTVQGHSCTCKPGYVGNGTICRGMPEVGPPRALLNSLDLGFLSFSKEALAVGMITLEGNIVAKSFTDDETLVERGREKVIALLFSWLHKEKLSLENLRDIYCKANSLVGLDHLPQR